(19) United States
(12) Patent Application Publication
(10) Pub. No.: US 2010/0093702 A1
(43) Pub. Date: Apr. 15, 2010

(54) METHYLENE AMINES OF THIENO[2,3-d]PYRIMIDINE AND THEIR USE AS ADENOSINE A2a RECEPTOR ANTAGONISTS

(76) Inventors: J. Kent Barbay, Flourtown, PA (US); Devraj Chakravarty, Hillsborough, NJ (US); Brian Christopher Shook, Gilbertsville, PA (US); Aihua Wang, Jamison, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/479,158

(22) Filed: Jun. 5, 2009

Related U.S. Application Data
(60) Provisional application No. 61/104,781, filed on Oct. 13, 2008.

(51) Int. Cl. A61K 31/55; C07D 409/14; A61K 31/519; C07D 405/14; C07D 413/14; A61K 31/5377; C07D 417/14; A61K 31/496; A61K 31/541; A61P 25/28; A61P 25/16; A61P 25/30; A61P 25/22; A61P 25/24

(52) U.S. Cl. 514/217.06; 544/278; 514/260.1; 544/117; 514/234.2; 514/252.16; 544/61; 514/228.5; 540/600

(57) ABSTRACT

This invention relates to a novel thieno[2,3-d]pyrimidine, A, and its therapeutic and prophylactic uses, wherein R¹ and R² are defined in the specification. Disorders treated and/or prevented include Parkinson's Disease.

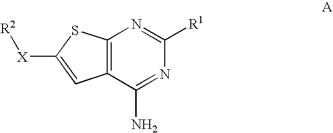

### CROSS-REFERENCE TO RELATED APPLICATIONS

The present application claims the benefits of the filing of U.S. Provisional Application No. 61/104,781 filed Oct. 13, 2008. The complete disclosures of the aforementioned related patent applications are hereby incorporated herein by reference for all purposes.

### FIELD OF THE INVENTION

This invention relates to a novel arylindenopyrimidine and its therapeutic and prophylactic uses. Disorders treated and/or prevented include neurodegenerative and movement disorders ameliorated by antagonizing Adenosine A2a receptors.

### BACKGROUND OF THE INVENTION

Adenosine A2a Receptors Adenosine is a purine nucleotide produced by all metabolically active cells within the body. Adenosine exerts its effects via four subtypes of cell surface receptors (A1, A2a, A2b and A3), which belong to the G protein coupled receptor superfamily (Stiles, G. L. Journal of Biological Chemistry, 1992, 267, 6451). A1 and A3 couple to inhibitory G protein, while A2a and A2b couple to stimulatory G protein. A2a receptors are mainly found in the brain, both in neurons and glial cells (highest level in the striatum and nucleus accumbens, moderate to high level in olfactory tubercle, hypothalamus, and hippocampus etc. regions) (Rosin, D. L.; Robeva, A.; Woodard, R. L.; Guyenet, P. G.; Linden, J. Journal of Comparative Neurology, 1998, 401, 163).

In peripheral tissues, A2a receptors are found in platelets, neutrophils, vascular smooth muscle and endothelium (Gessi, S.; Varani, K.; Merighi, S.; Ongini, E.; Bores, P. A. British Journal of Pharmacology, 2000, 129, 2). The striatum is the main brain region for the regulation of motor activity, particularly through its innervation from dopaminergic neurons originating in the substantial nigra. The striatum is the major target of the dopaminergic neuron degeneration in patients with Parkinson's Disease (PD). Within the striatum, A2a receptors are co-localized with dopamine D2 receptors, suggesting an important site for the integration of adenosine and dopamine signaling in the brain (Fink, J. S.; Weaver, D. Ri; Rivkees, S. A.; Peterfreund, R. A.; Pollack, A. E.; Adler, E. M.; Reppert, S. M. Brain Research Molecular Brain Research, 1992, 14, 186).

Neurochemical studies have shown that activation of A2a receptors reduces the binding affinity of D2 agonist to their receptors. This D2R and A2aR receptor-receptorinteraction has been demonstrated instriatal membrane preparations of rats (Ferre, S.; con Euler, G.; Johansson, B.; Fredholm, B. B.; Fuxe, K. Proceedings of the National Academy of Sciences I of the United States of America, 1991, 88, 7238) as well as in fibroblast cell lines after transfected with A2aR and D2R cDNAs (Salim, H.; Ferre, S.; Dalal, A.; Peterfreund, R. A.; Fuxe, K.; Vincent, J. D.; Lledo, P. M. Journal of Neurochemistry, 2000, 74, 432). In vivo, pharmacological blockade of A2a receptors using A2a antagonist leads to beneficial effects in dopaminergic neurotoxin MPTP (1-methyl-4-pheny-1,2,3,6-tetrahydropyridine)-induced PC) in various species, including mice, rats, and monkeys (Ikeda, K.; Kurokawa, M.; Aoyana, S.; Kuwana, Y. Journal of Neurochemistry, 2002, 80, 262).

Furthermore, A2a knockout mice with genetic blockade of A2a function have been found to be less sensitive to motor impairment and neurochemical changes when they were exposed to neurotoxin MPTP (Chen, J. F.; Xu, K.; I Petzer, J. P.; Steal, R.; Xu, Y. H.; Beilstein, M.; Sonsalla, P. K.; Castagnoli, K.; Castagnoli, N., Jr.; Schwarsschild, M. A. Journal of Neuroscience, 2001, 1 21, RC1 43).

In humans, the adenosine receptor antagonist theophylline has been found to produce beneficial effects in PD patients (Mally, J.; Stone, T. W. Journal of the Neurological Sciences, 1995, 132, 129). Consistently, recent epidemiological study has shown that high caffeine consumption makes people less likely to develop PD (Ascherio, A.; Zhang, S. M.; Heman, M. A.; Kawachi, I.; Colditz, G. A.; Speizer, F. E.; Willett, W. C. Annals of Neurology, 2001, 50, 56). In summary, adenosine A2a receptor blockers may provide a new class of antiparkinsonian agents (Impagnatiello, F.; Bastia, E.; Ongini, E.; Monopoli, A. Emerging Therapeutic Targets, 2000, 4, 635).

Antagonists of the A_{2A} receptor are potentially useful therapies for the treatment of addiction. Major drugs of abuse (opiates, cocaine, ethanol, and the like) either directly or indirectly modulate dopamine signaling in neurons particularly those found in the nucleus accumbens, which contain high levels of A_{2A} adenosine receptors. Dependence has been shown to be augmented by the adenosine signaling pathway, and it has been shown that administration of an A_{2A} receptor antagonist redues the craving for addictive substances (“The Critical Role of Adenosine A_{2A} Receptors and Gi βγ Subunits in Alcoholism and Addiction: From Cell Biology to Behavior”, by Ivan Diamond and Lina Yao, (The Cell Biology of Addiction, 2006, pp 291-316) and “Adaptations in Adenosine Signaling in Drug Dependence: Therapeutic Implications”, by Stephen P. Hack and Macdonald J. Christie, Critical Review in Neurobiology, Vol. 15, 235-274 (2003)). See also Alcoholism: Clinical and Experimental Research (2007), 31(8), 1302-1307.

An A_{2A} receptor antagonist could be used to treat attention deficit hyperactivity disorder (ADHD) since caffeine (a non selective adenosine antagonist) can be useful for treating ADHD, and there are many interactions between dopamine and adenosine neurons. Clinical Genetics (2000), 58(1), 31-40 and references therein.

Antagonists of the A_{2A} receptor are potentially useful therapies for the treatment of depression. A_{2A} antagonists are known to induce activity in various models of depression including the forced swim and tail suspension tests. The positive response is mediated by dopaminergic transmission and is caused by a prolongation of escape-directed behavior rather than by a motor stimulant effect. Neurology (2003), 61(suppl 6) S82-S87.

Antagonists of the A_{2A} receptor are potentially useful therapies for the treatment of anxiety. A_{2A} antagonist have been shown to prevent emotional/anxious responses in vivo. Neurobiology of Disease (2007), 28(2) 197-205.

### SUMMARY OF THE INVENTION

The present invention includes compounds of Formula A

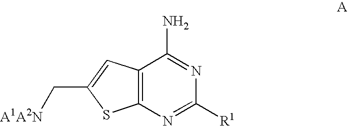

wherein:
R¹ is cyclopropyl, benzo[1,3]dioxolyl, or R¹ is phenyl wherein said phenyl is optionally substituted with up to three substituents independently selected from the group consisting of F, Cl, Br, and OCH₃, or a single substituent selected from the group consisting of: OH, OCH₂CF₃, OC₍₁₋₄₎alkyl, C₍₁₋₄₎alkyl, CHF₂, OCF₃, CF₃, and CN; or R¹ is heteroaryl optionally substituted with one substituent selected from the group consisting of: —OH, OC₍₁₋₄₎alkyl, CF₃, OCF₃, Cl, Br, —CN, F, CHF₂, and C₍₁₋₄₎alkyl;
A¹ is H or —C₍₁₋₄₎alkyl;
A² is —C₍₁₋₄₎alkyl, —C₍₁₋₆₎cycloalkyl, —CH₂CH₂OR^{a}, —COR^{a}, heteroaryl, adamantyl, or phenyl, wherein said heteroaryl or phenyl is optionally substituted with up to three substituents selected from the group consisting of Cl, F, Br, OC₍₁₋₄₎alkyl, OCF₃, C₍₁₋₄₎alkyl, and C(O)C₍₁₋₄₎alkyl;
alternatively, A¹ and A² may be taken together with their attached nitrogen to form a heterocyclic ring selected from the group consisting of:

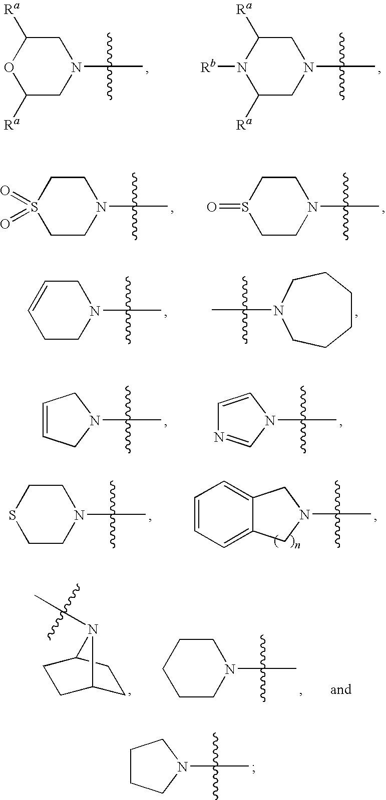

wherein said

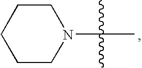

and said

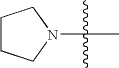

are optionally substituted with R^{a}, R^{c}, oxo, phenyl, or CH₂OC₍₁₋₄₎alkyl;
wherein:
n is 1 or 2;
R^{a} is H, CF₃, OH, F, or C₍₁₋₄₎alkyl;
R^{b} is H, —C₍₁₋₄₎alkyl, or —C(O)C₍₁₋₄₎alkyl; and
R^{c} is H or F;
   and solvates, hydrates, and pharmaceutically acceptable salts thereof.

### DETAILED DESCRIPTION OF THE INVENTION

The present invention includes compounds of Formula A

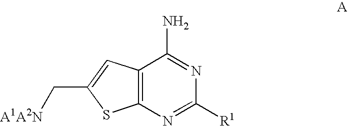

wherein:
R¹ is cyclopropyl, benzo[1,3]dioxolyl, or R¹ is phenyl wherein said phenyl is optionally substituted with up to three substituents independently selected from the group consisting of F, Cl, Br, and OCH₃, or a single substituent selected from the group consisting of: OH, OCH₂CF₃, OC₍₁₋₄₎alkyl, C₍₁₋₄₎alkyl, CHF₂, OCF₃, CF₃, and CN; or R¹ is heteroaryl optionally substituted with one substituent selected from the group consisting of: —OH, OC₍₁₋₄₎alkyl, CF₃, OCF₃, Cl, Br, —CN, F, CHF₂, and C₍₁₋₄₎alkyl;
A¹ is H or —C₍₁₋₄₎alkyl;
A² is —C₍₁₋₄₎alkyl, —C₍₁₋₆₎cycloalkyl, —CH₂CH₂OR^{a}, —COR^{a}, heteroaryl, adamantyl, or phenyl, wherein said heteroaryl or phenyl is optionally substituted with up to three substituents selected from the group consisting of Cl, F, Br, OC₍₁₋₄₎alkyl, OCF₃, C₍₁₋₄₎alkyl, and C(O)C₍₁₋₄₎alkyl;
alternatively, A¹ and A² may be taken together with their attached nitrogen to form a heterocyclic ring selected from the group consisting of:

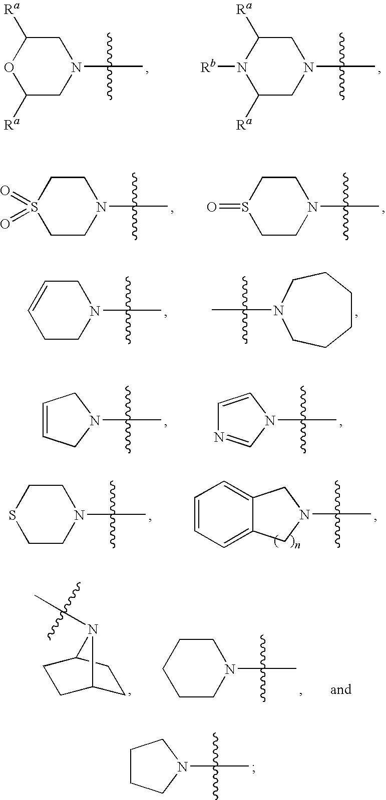

wherein said

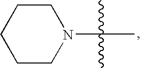

and said

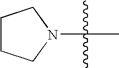

are optionally substituted with R^{a}, R^{c}, oxo, phenyl, or CH₂OC₍₁₋₄₎alkyl;
wherein:
n is 1 or 2;
R^{a} is H, CF₃, OH, F, or C₍₁₋₄₎alkyl;
R^{b} is H, —C₍₁₋₄₎alkyl, or —C(O)C₍₁₋₄₎alkyl; and
R^{c} is H or F;
   and solvates, hydrates, and pharmaceutically acceptable salts thereof.

In another embodiment of the invention:

R¹ is cyclopropyl, furyl, thiazolyl, thiophenyl, oxazolyl, isoxazolyl, pyridyl, benzo[1,3]dioxolyl, pyrrolyl, benzofuranyl, fluorophenyl, or phenyl, wherein said furyl, thiazolyl, thiophenyl, oxazolyl, isoxazolyl, pyridyl, benzo[1,3]dioxolyl, pyrrolyl, benzofuranyl, or phenyl is optionally substituted with OH, OC₍₁₋₄₎alkyl, Cl, Br, —CN, F, CHF₂, OCF₃, C₍₁₋₄₎alkyl, or cyclopropyl;
A¹ is H or —C₍₁₋₄₎alkyl;
A² is —C₍₁₋₄₎alkyl, —C₍₁₋₆₎cycloalkyl, —CH₂CH₂OR^{a}, —COR^{a}, heteroaryl, adamantyl, or phenyl, wherein said heteroaryl or phenyl is optionally substituted with up to three substituents selected from the group consisting of Cl, F, Br, OC₍₁₋₄₎alkyl, OCF₃, C₍₁₋₄₎alkyl, and C(O)C₍₁₋₄₎alkyl;
alternatively, A¹ and A² may be taken together with their attached nitrogen to form a heterocyclic ring selected from the group consisting of:

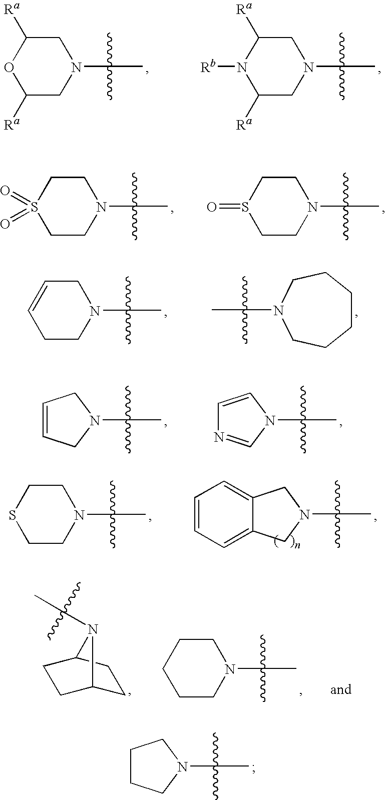

wherein said

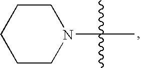

and said

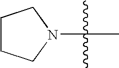

are optionally substituted with R^{a}, R^{c}, oxo, phenyl, or CH₂OC₍₁₋₄₎alkyl;
wherein:
n is 1 or 2;
R^{a} is H, CF₃, OH, F, or C₍₁₋₄₎alkyl;
R^{b} is H, —C₍₁₋₄₎alkyl, or —C(O)C₍₁₋₄₎alkyl; and
R^{c} is H or F;
   and solvates, hydrates, and pharmaceutically acceptable salts thereof.

In another embodiment of the invention:

R¹ is cyclopropyl, furyl, thiazolyl, thiophenyl, oxazolyl, isoxazolyl, pyridyl, benzo[1,3]dioxolyl, pyrrolyl, benzofuranyl, fluorophenyl, or phenyl, wherein said furyl, thiazolyl, thiophenyl, oxazolyl, isoxazolyl, pyridyl, benzo[1,3]dioxolyl, pyrrolyl, benzofuranyl, or phenyl is optionally substituted with OH, OC₍₁₋₄₎alkyl, Cl, Br, —CN, F, CHF₂, OCF₃, C₍₁₋₄₎alkyl, or cyclopropyl;
A¹ is H or —C₍₁₋₄₎alkyl;
A² is —C₍₁₋₄₎alkyl, —C₍₁₋₆₎cycloalkyl, —CH₂CH₂OR^{a}, —COR^{a}, pyridyl, adamantyl, or phenyl, wherein said heteroaryl or phenyl is optionally substituted with up to three substituents selected from the group consisting of Cl, F, Br, OC₍₁₋₄₎alkyl, OCF₃, C₍₁₋₄₎alkyl, and C(O)C₍₁₋₄₎alkyl;
alternatively, A¹ and A² may be taken together with their attached nitrogen to form a heterocyclic ring selected from the following:

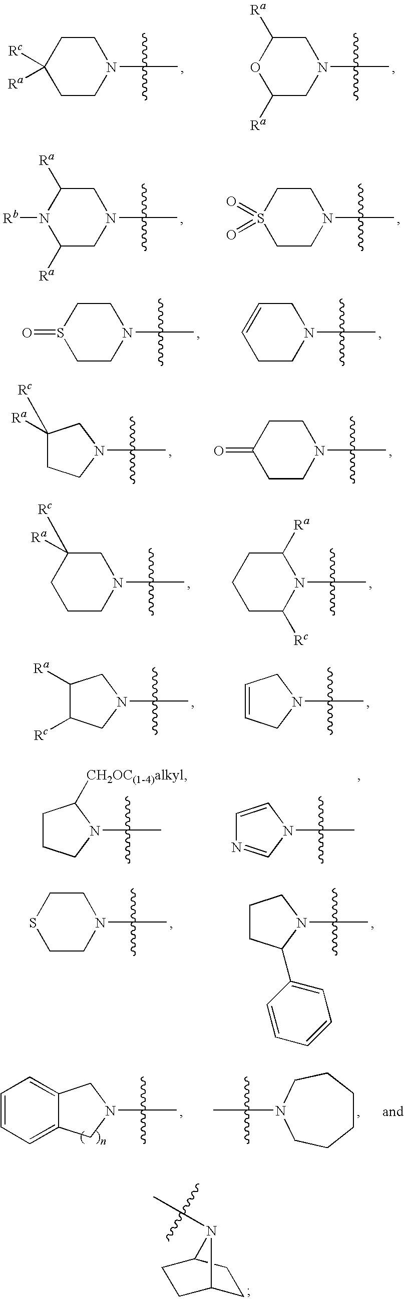

wherein:
n is 1 or 2
R^{a} is H, CF₃, OH, F, or C₍₁₋₄₎alkyl;
R^{b} is H, —C₍₁₋₄₎alkyl, or —C(O)C₍₁₋₄₎alkyl; and
R^{c} is H or F;
   and solvates, hydrates, and pharmaceutically acceptable salts thereof.

In another embodiment of the invention:

R¹ is cyclopropyl, furyl, thiazolyl, thiophenyl, oxazolyl, isoxazolyl, pyridyl, benzo[1,3]dioxolyl, pyrrolyl, benzofuranyl, fluorophenyl, or phenyl, wherein said furyl, thiazolyl, thiophenyl, oxazolyl, isoxazolyl, pyridyl, benzo[1,3]dioxolyl, pyrrolyl, benzofuranyl, fluorophenyl, or phenyl is optionally substituted with OH, OCH₃, Cl, Br, —CN, F, CHF₂, OCF₃, CH₃, CH₂CH₃, CH(CH₃)₂, C(CH₃)₃, or cyclopropyl;
A¹ is H, or C₍₁₋₄₎alkyl;
A² is C₍₁₋₄₎alkyl, —CH₂CH₂OCH₃, cyclopropyl, adamantyl, or cyclohexyl;
alternatively, A¹ and A² may be taken together with their attached nitrogen to form a heterocyclic ring selected from the following:

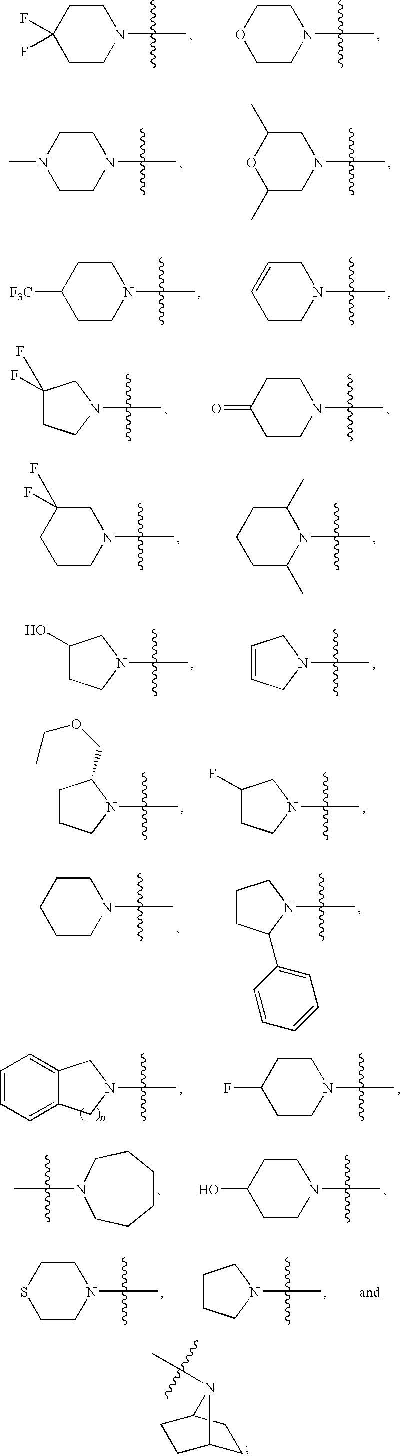

wherein n is 1 or 2;
and solvates, hydrates, and pharmaceutically acceptable salts thereof.

In another embodiment of the invention:

R¹ is cyclopropyl; furyl, wherein said furyl is optionally substituted with Cl, Br, cyclopropyl, CH₃, CH₂CH₃, CHF₂, or CH(CH₃)₂; thiazolyl, wherein said thiazolyl is optionally substituted with CH₃; thiophenyl, wherein said thiophenyl is optionally substituted with C(CH₃)₃, or —CN; oxazolyl; isoxazolyl; pyridyl, wherein said pyridyl is substituted with —CN, or Cl; benzo[1,3]dioxolyl, pyrrolyl, wherein said pyrrolyl is optionally substituted with CH₃; benzofuranyl, fluorophenyl, wherein said fluorophenyl is optionally substituted with F; or phenyl, wherein said phenyl is substituted with CN, Cl, OCH₃, CON(CH₃)₂, CH(CH₃)₂, or OH;

### A¹ is H, —CH₃, or —CH₂CH₃;

A² is —CH₃, —CH₂CH₃, —CH₂CH₂OCH₃, cyclopropyl, adamantyl, or cyclohexyl;
alternatively, A¹ and A² may be taken together with their attached nitrogen to form a heterocyclic ring selected from the following:

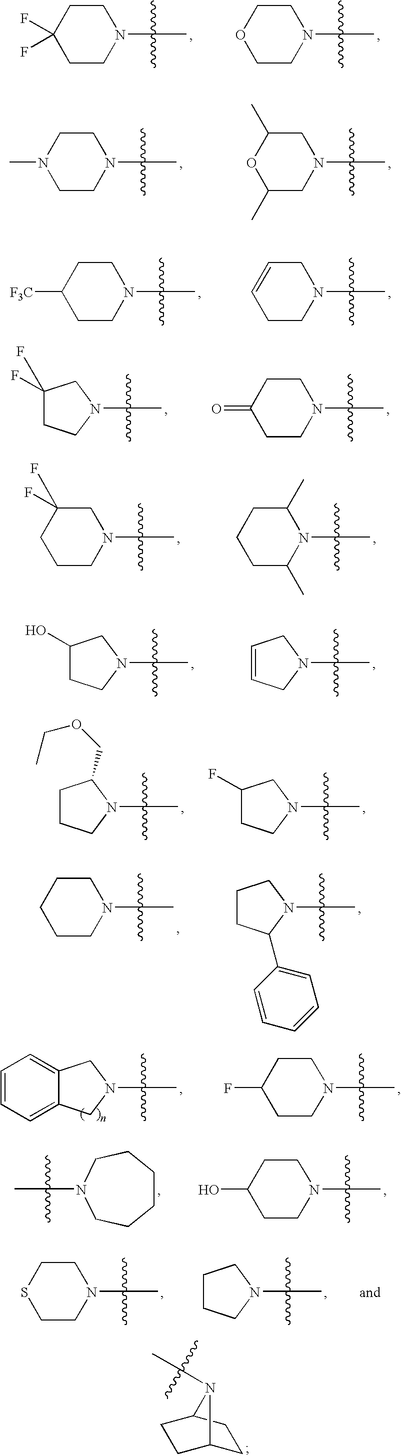

wherein n is 1 or 2;
and solvates, hydrates, and pharmaceutically acceptable salts thereof.

Another embodiment of the invention comprises a compound selected from the group consisting of:

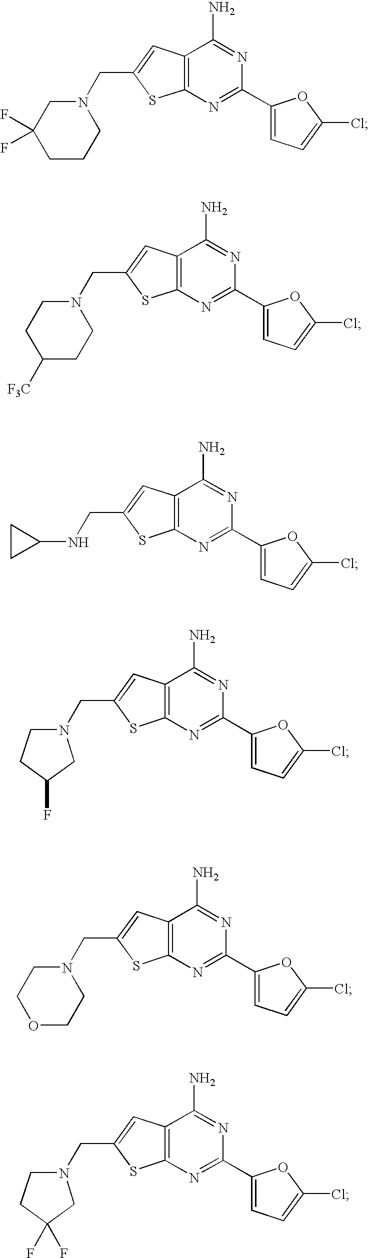

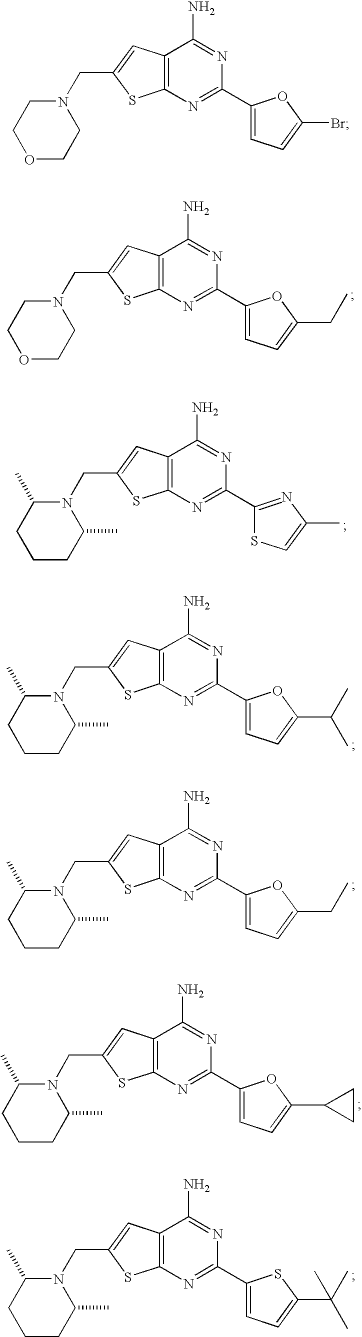

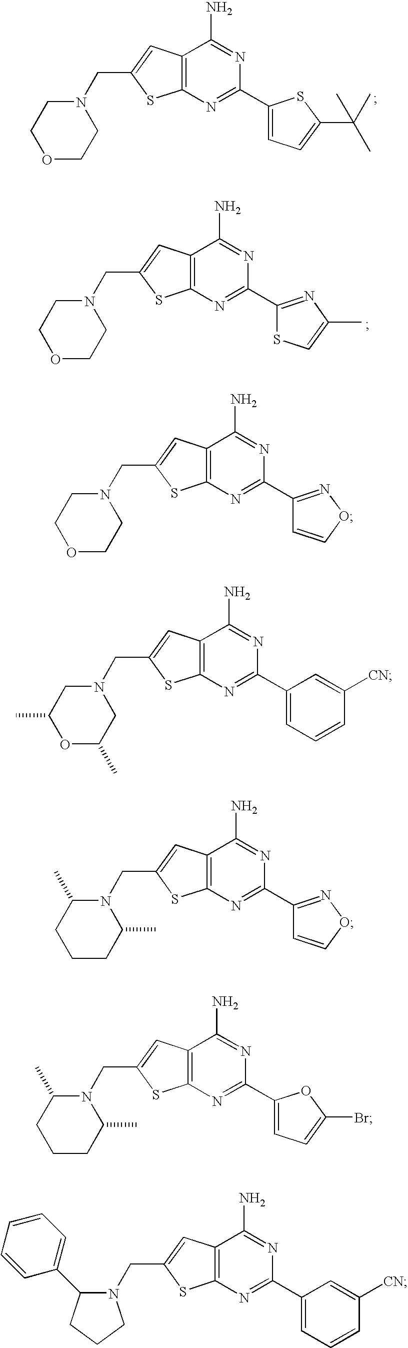

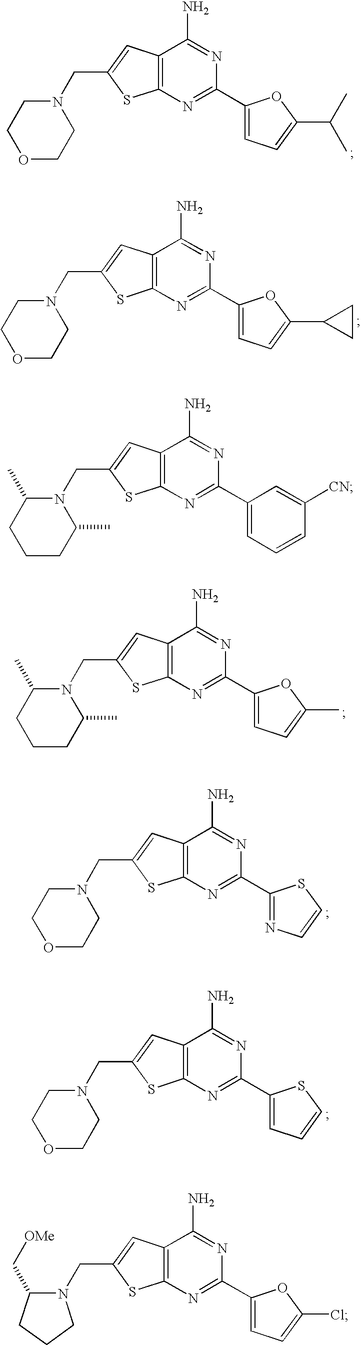

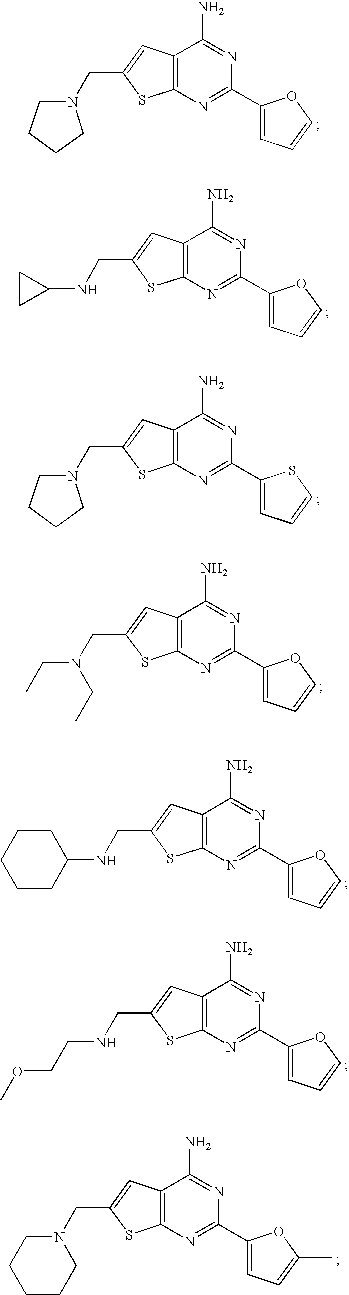

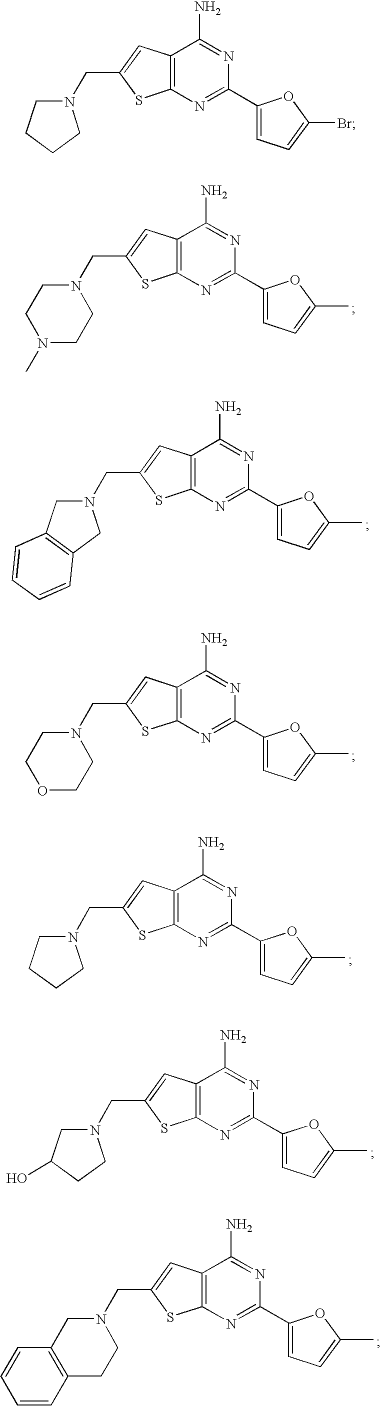

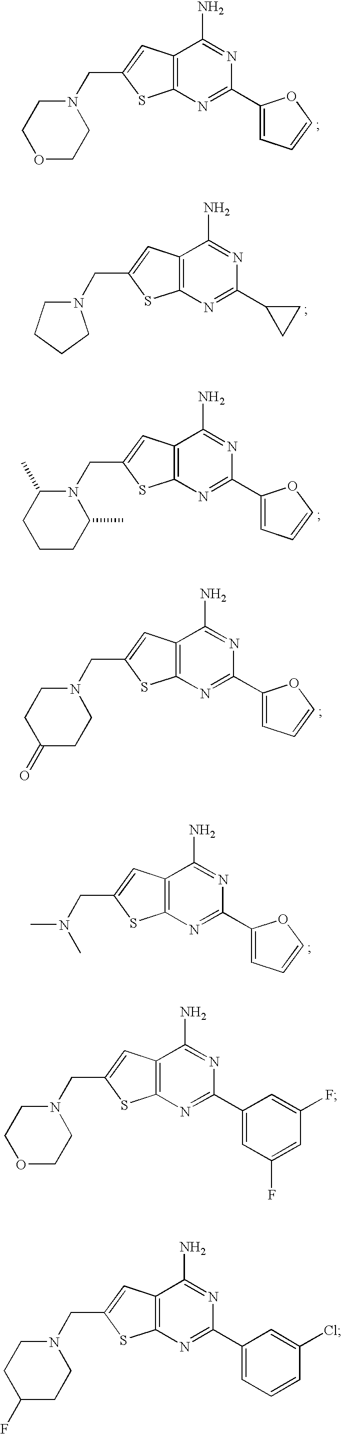

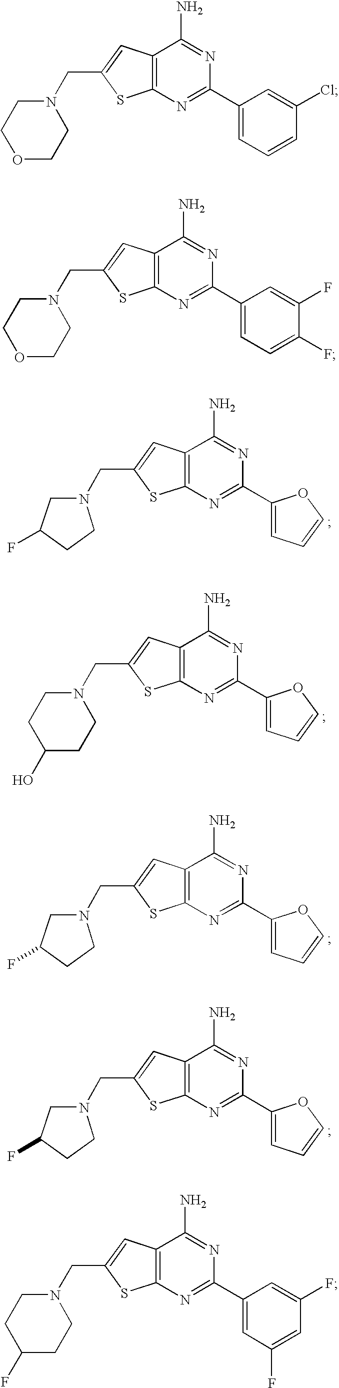

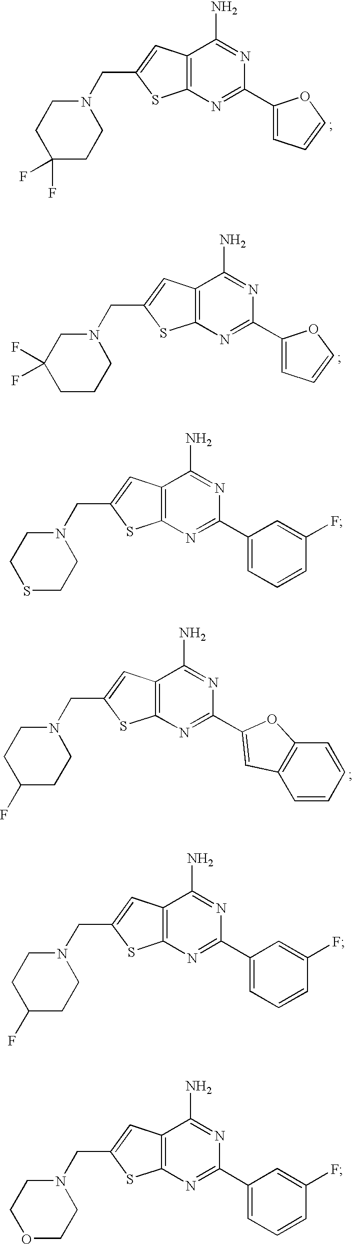

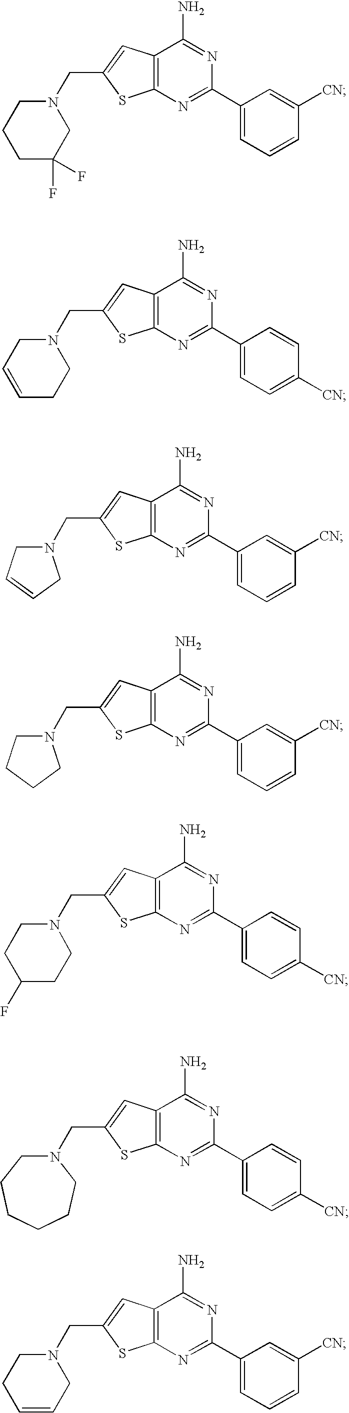

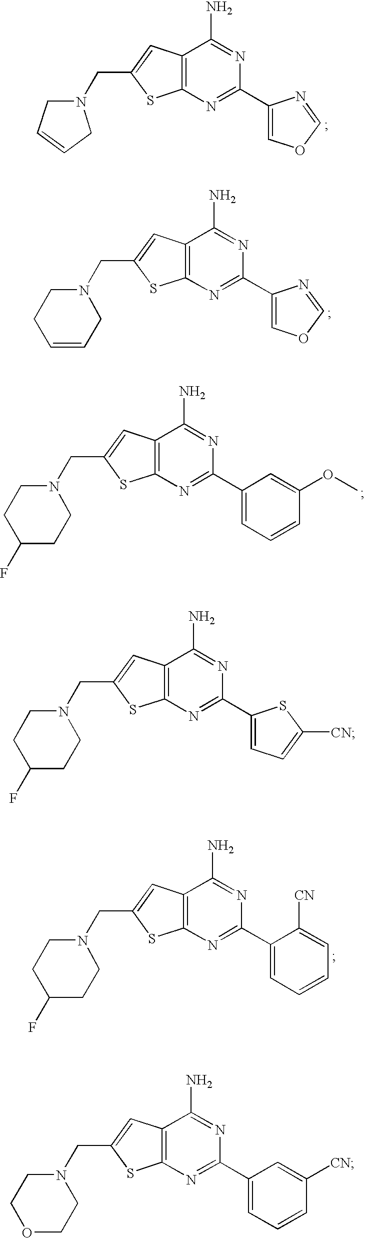

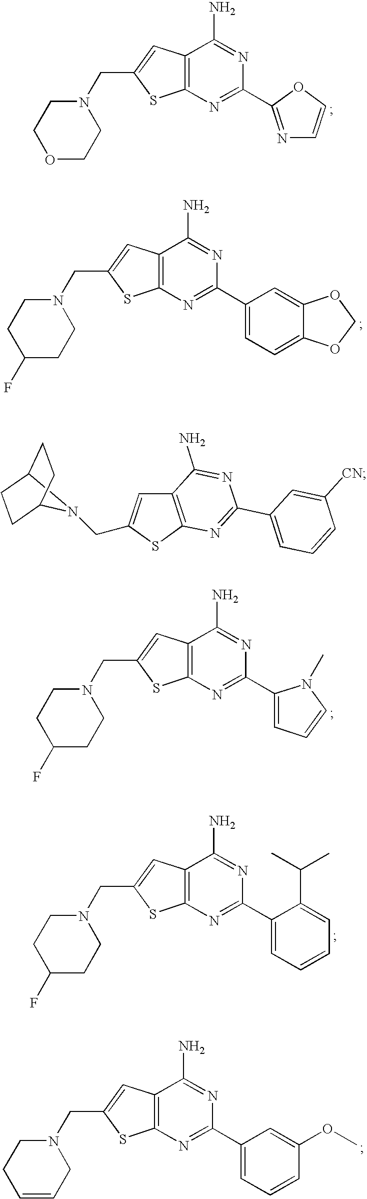

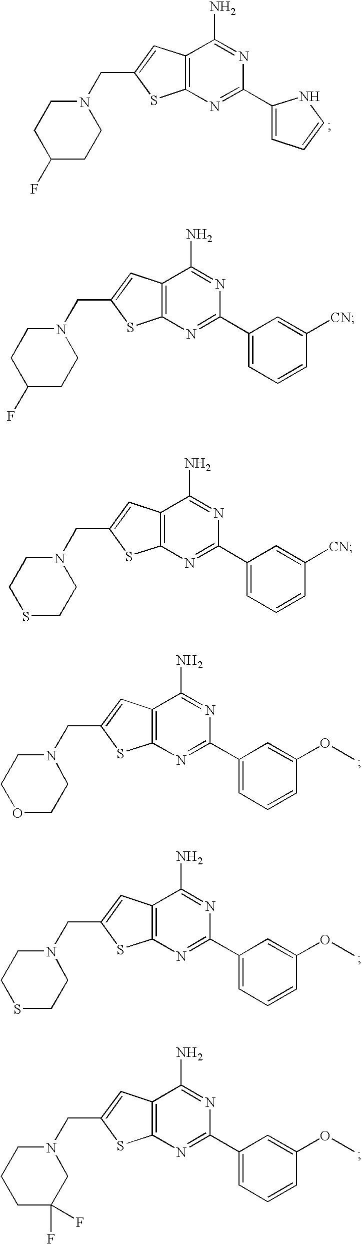

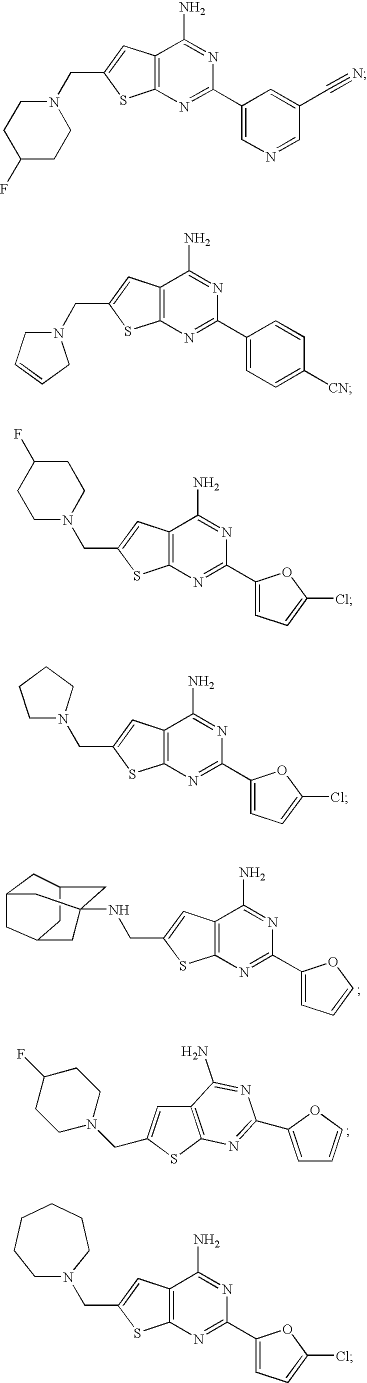

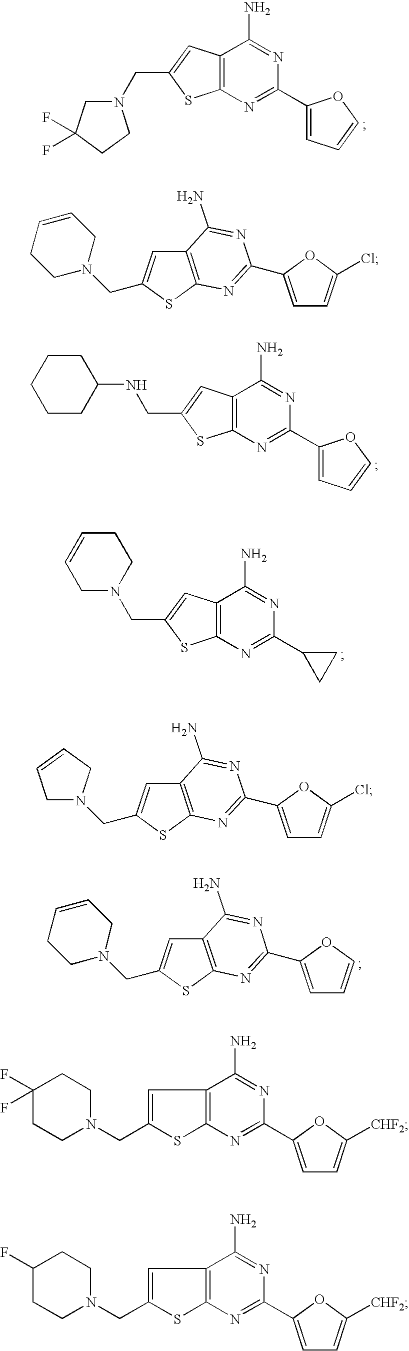

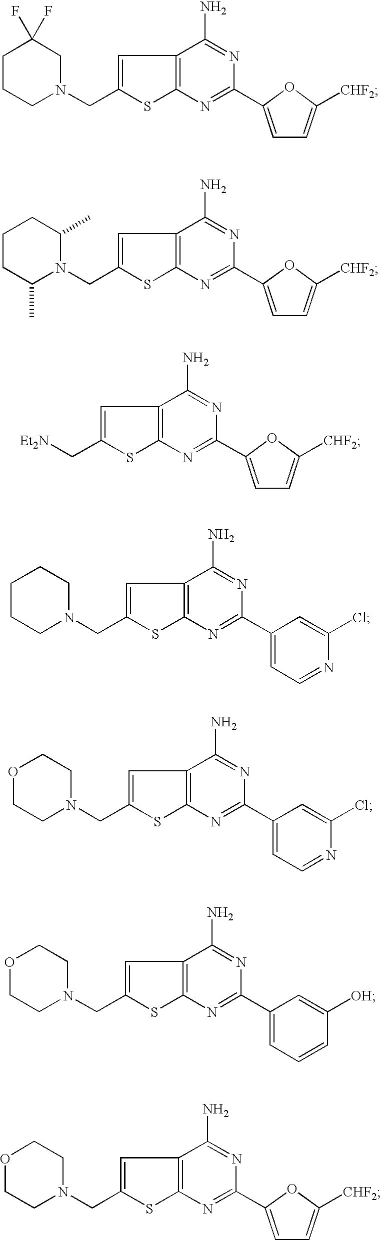

and solvates, hydrates, and pharmaceutically acceptable salts thereof.

This invention further provides a method of treating a subject having a condition ameliorated by antagonizing Adenosine A2a receptors, which comprises administering to the subject a therapeutically effective dose of a compound of Formula A.

This invention further provides a method of preventing a disorder ameliorated by antagonizing Adenosine A2a receptors in a subject, comprising of administering to the subject a prophylactically effective dose of the compound of claim 1 either preceding or subsequent to an event anticipated to cause a disorder ameliorated by antagonizing Adenosine A2a receptors in the subject.

Compounds of Formula A can be isolated and used as free bases. They can also be isolated and used as pharmaceutically acceptable salts.

Examples of such salts include hydrobromic, hydroiodic, hydrochloric, perchloric, sulfuric, maleic, fumaric, malic, tartaric, citric, adipic, benzoic, mandelic, methanesulfonic, hydroethanesulfonic, benzenesulfonic, oxalic, palmoic, 2 naphthalenesulfonic, p-toluenesulfonic, cyclohexanesulfamic and saccharic.

This invention also provides a pharmaceutical composition comprising a compound of Formula A and a pharmaceutically acceptable carrier.

Pharmaceutically acceptable carriers are well known to those skilled in the art and include, but are not limited to, from about 0.01 to about 0.1 M and preferably 0.05 M phosphate buyer or 0.8% saline. Such pharmaceutically acceptable carriers can be aqueous or non-aqueous solutions, suspensions and emulsions. Examples of non-aqueous solvents are propylene glycol, polyethylene glycol, vegetable oils such as olive oil, and injectable organic esters such as ethyl oleate. Aqueous carriers include water, ethanol, alcoholic/aqueous solutions, glycerol, emulsions or suspensions, including saline and buffered media. Oral carriers can be elixirs, syrups, capsules, tablets and the like. The typical solid carrier is an inert substance such as lactose, starch, glucose, methyl-cellulose, magnesium stearate, dicalcium phosphate, mannitol and the like. Parenteral carriers include sodium chloride solution, Ringer's dextrose, dextrose and sodium chloride, lactated Ringer's and fixed oils. Intravenous carriers include fluid and nutrient replenishers, electrolyte replenishers such as those based on Ringer's dextrose and the like.

Preservatives and other additives can also be present, such as, for example, antimicrobials, antioxidants, chelating agents, inert gases and the like. All carriers can be mixed as needed with disintegrants, diluents, granulating agents, lubricants, binders and the like using conventional techniques known in the art.

This invention further provides a method of treating a subject having a condition ameliorated by antagonizing Adenosine A2a receptors, which comprises administering to the subject a therapeutically effective dose of a compound of Formula A.

In one embodiment, the disorder is a neurodegenerative or movement disorder. Examples of disorders treatable by the instant pharmaceutical composition include, without limitation, Parkinson's Disease, Huntington's Disease, Multiple System Atrophy, Corticobasal Degeneration, Alzheimer's Disease, and Senile Dementia.

In one preferred embodiment, the disorder is Parkinson's disease.

As used herein, the term “subject” includes, without limitation, any animal or artificially modified animal having a disorder ameliorated by antagonizing adenosine A2a receptors. In a preferred embodiment, the subject is a human.

Administering the instant pharmaceutical composition can be effected or performed using any of the various methods known to those skilled in the art. Compounds of Formula A can be administered, for example, intravenously, intramuscularly, orally and subcutaneously. In the preferred embodiment, the instant pharmaceutical composition is administered orally. Additionally, administration can comprise giving the subject a plurality of dosages over a suitable period of time. Such administration regimens can be determined according to routine methods.

As used herein, a “therapeutically effective dose” of a pharmaceutical composition is an amount sufficient to stop, reverse or reduce the progression of a disorder. A “prophylactically effective dose” of a pharmaceutical composition is an amount sufficient to prevent a disorder, i.e., eliminate, ameliorate and/or delay the disorder's onset. Methods are known in the art for determining therapeutically and prophylactically effective doses for the instant pharmaceutical composition. The effective dose for administering the pharmaceutical composition to a human, for example, can be determined mathematically from the results of animal studies.

In one embodiment, the therapeutically and/or prophylactically effective dose is a dose sufficient to deliver from about 0.001 mg/kg of body weight to about 200 mg/kg of body weight of a compound of Formula A. In another embodiment, the therapeutically and/or prophylactically effective dose is a dose sufficient to deliver from about 0.05 mg/kg of body weight to about 50 mg/kg of body weight. More specifically, in one embodiment, oral doses range from about 0.05 mg/kg to about 100 mg/kg daily. In another embodiment, oral doses range from about 0.05 mg/kg to about 50 mg/kg daily, and in a further embodiment, from about 0.05 mg/kg to about 20 mg/kg daily. In yet another embodiment, infusion doses range from about 1.0, ug/kg/min to about 10 mg/kg/min of inhibitor, admixed with a pharmaceutical carrier over a period ranging from about several minutes to about several days. In a further embodiment, for topical administration, the instant compound can be combined with a pharmaceutical carrier at a drug/carrier ratio of from about 0.001 to about 0.1.

The invention also provides a method of treating addiction in a mammal, comprising administering a therapeutically effective dose of a compound of Formula A.

The invention also provides a method of treating ADHD in a mammal, comprising administering a therapeutically effective dose of a compound of Formula A.

The invention also provides a method of treating depression in a mammal, comprising administering a therapeutically effective dose of a compound of Formula A.

The invention also provides a method of treating anxiety in a mammal, comprising administering a therapeutically effective dose of a compound of Formula A.

### DEFINITIONS

The term “C_{a-b}” (where a and b are integers referring to a designated number of carbon atoms) refers to an alkyl, alkenyl, alkynyl, alkoxy or cycloalkyl radical or to the alkyl portion of a radical in which alkyl appears as the prefix root containing from a to b carbon atoms inclusive. For example, C₁₋₄ denotes a radical containing 1, 2, 3 or 4 carbon atoms.

The term “adamantyl” refers to the following radical

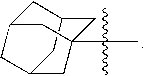

The term “alkyl,” whether used alone or as part of a substituent group, refers to a saturated branched or straight chain monovalent hydrocarbon radical, wherein the radical is derived by the removal of one hydrogen atom from a single carbon atom. Unless specifically indicated (e.g. by the use of a limiting term such as “terminal carbon atom”), substituent variables may be placed on any carbon chain atom. Typical alkyl radicals include, but are not limited to, methyl, ethyl, propyl, isopropyl and the like. Examples include C₁₋₈alkyl, C₁₋₆alkyl and C₁₋₄alkyl groups.

The term “benzo[1,3]dioxolyl” refers to the following radical

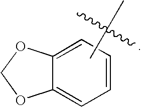

The term “heteroaryl” refers to a radical derived by the removal of one hydrogen atom from a ring carbon atom of a heteroaromatic ring system. Typical heteroaryl radicals include furyl, pyrrolyl, oxazolyl, thiophenyl, thiazolyl, imidazolyl, pyrazolyl, isoxazolyl, isothiazolyl, oxadiazolyl, triazolyl, thiadiazolyl, pyridinyl, pyridazinyl, pyrimidinyl, pyrazinyl, indolizinyl, indolyl, isoindolyl, indazolyl, benzimidazolyl, benzothiazolyl, purinyl, 4H-quinolizinyl, quinolinyl, isoquinolinyl, cinnolinyl, phthalzinyl, quinazolinyl, quinoxalinyl, 1,8-naphthyridinyl, pteridinyl and the like.

The term “heterocyclyl” refers to a radical derived by the removal of one hydrogen atom from a ring carbon or ring nitrogen atom of a saturated or partially saturated heteroaromatic ring system. Typical heterocyclyl radicals include morpholinyl, piperidinyl, piperazinyl, pyrrolidinyl, tetrahydrofuranyl, and the like.

The term “oxo” refers to a substitution available to a methylene group wherein both C—H bonds have been replaced by bonds to the same oxygen. For example, acetone is an oxo substituted propane.

### ABBREVIATIONS

Herein and throughout this application, the following abbreviations may be used.

Cy cyclohexyl
DMF dimethylformamide
DMSO dimethylsulfoxide
Et ethyl
EtOAc ethyl acetate
KOtBu potassium tert-butoxide
Me methyl
NBS N-bromo succinimide
OAc acetate
Pd(dppf)Cl₂ [1,1′-Bis(diphenylphosphino)ferrocene]dichloropalladium (II)
py pyridine
THF tetrahydrofuran
Xantphos 9,9-Dimethyl-4,5-bis(diphenylphosphino)xanthene

The present invention includes within its scope prodrugs of the compounds of this invention. In general, such prodrugs will be functional derivatives of the compounds which are readily convertible in vivo into the required compound. Thus, in the methods of treatment of the present invention, the term “administering” shall encompass the treatment of the various disorders described with the compound specifically disclosed or with a compound which may not be specifically disclosed, but which converts to the specified compound in vivo after administration to the patient. Conventional procedures for the selection and preparation of suitable prodrug derivatives are described, for example, in “Design of Prodrugs”, Ed. H. Bundgaard, Elsevier, 1985.

Where the compounds according to this invention have at least one chiral center, they may accordingly exist as enantiomers. Where the compounds possess two or more chiral centers, they may additionally exist as diastereomers. It is to be understood that all such isomers and mixtures thereof are encompassed within the scope of the present invention.

Where the processes for the preparation of the compounds according to the invention give rise to mixture of stereoisomers, these isomers may be separated by conventional techniques such as preparative chromatography. The compounds may be prepared in racemic form, or individual enantiomers may be prepared either by enantiospecific synthesis or by resolution. The compounds may, for example, be resolved into their component enantiomers by standard techniques, such as the formation of diastereomeric pairs by salt formation with an optically active acid, such as (−)-di-p-toluoyl-D-tartaric acid and/or (+)-di-p-toluoyl-L-tartaric acid followed by fractional crystallization and regeneration of the free base. The compounds may also be resolved by formation of diastereomeric esters or amides, followed by chromatographic separation and removal of the chiral auxiliary. Alternatively, the compounds may be resolved using a chiral HPLC column.

During any of the processes for preparation of the compounds of the present invention, it may be necessary and/or desirable to protect sensitive or reactive groups on any of the molecules concerned. This may be achieved by means of conventional protecting groups, such as those described in Protective Groups in Organic Chemistry, ed. J. F. W. McOmie, Plenum Press, 1973; and T. W. Greene & P. G. M. Wuts, Protective Groups in Organic Synthesis, John Wiley & Sons, 1991. The protecting groups may be removed at a convenient subsequent stage using methods known from the art.

### General Schemes:

Compounds of formula A can be prepared by methods known to those who are skilled in the art. The following reaction schemes are only meant to represent examples of the invention and are in no way meant to be a limit of the invention.

### Procedure

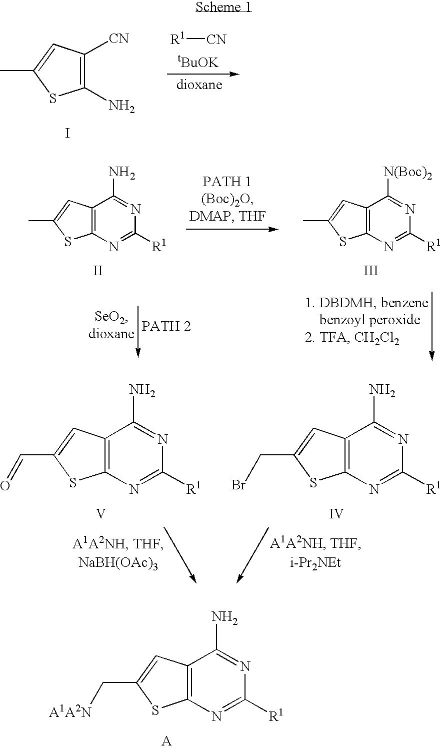

Scheme 1 illustrates the synthetic routes (Paths 1 and 2) leading to compounds of formula A. Starting with 2-amino-5-methyl-thiophene-3-carbonitrile I, condensation under basic conditions with R¹—CN, where R¹ is as defined in formula A, affords the aminopyrimidine II. Following path 1, the aminopyrimidine II is reacted with di-tert-butyldicarbonate [(Boc)₂O] in the presence of 4-dimethylamino pyridine (DMAP) to give the corresponding protected amine III. Methylthiophene III can undergo radical bromination using 1,3-dibromo-5,5-dimethylhydantoin (DBDMH) followed by deprotection using trifluoroacetic acid (TFA) to give the bromide IV. Displacement of the bromide is accomplished using A¹A²NH, where A¹ and A² are as defined in formula A, to give compounds of the formula A. Alternatively, following path 2, aminopyrimidine II can react with selenium dioxide (SeO₂) to give the corresponding aldehyde V that can then undergo reductive amination using A¹A²NH, where A¹ and A² are as defined in formula A, to give compounds of the formula A.

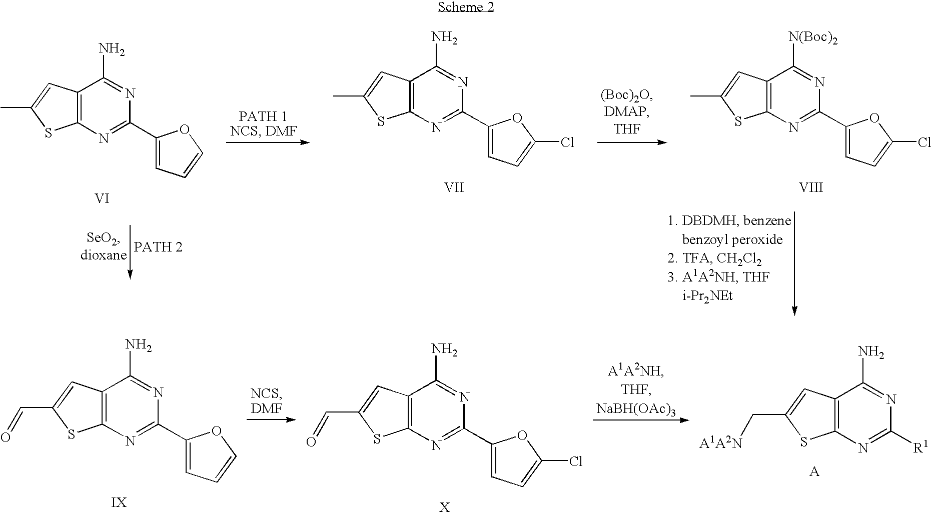

Scheme 2 illustrates the synthetic routes (Paths 1 and 2) leading to compounds of the formula A, where R¹=5-chloro-furan-2-yl. Starting with aminopyrimidine VI, obtained from condensing 2-amino-5-methyl-thiophene-3-carbonitrile I with 2-furonitrile as outlined in scheme 1, following path 1, is reacted with N-chlorosuccinimide (NCS) to give the chlorofuran VII. The chlorofuran VII is reacted with (Boc)₂O in the presence of DMAP to give the corresponding protected amine VIII. Compound VIII is brominated, deprotected, and alkylated in the same manner as described in scheme 1 to give compounds of formula A where R¹=5-chloro-furan-2-yl. Alternatively, following path 2, aminopyrimidine VI can react with selenium dioxide (SeO₂) to give the corresponding aldehyde IX that is then reacted with NCS to give chloride X that can then undergo reductive amination using A¹A²NH as described in scheme 1 to give compounds of formula A where R¹=5-chloro-furan-2-yl.

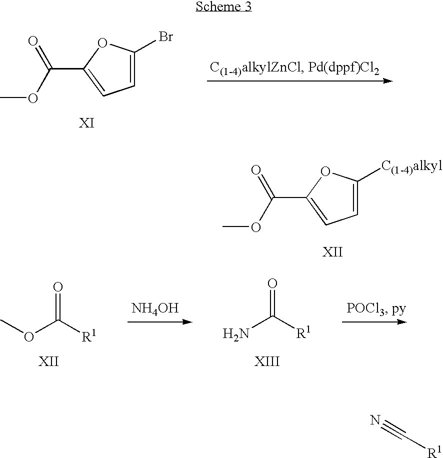

Scheme 3 illustrates the synthetic route to compounds of Formula R¹—CN, where R¹ is a C₍₁₋₄₎alkyl substituted furan. Scheme 3 also illustrates how any R¹—CO₂CH₃ may be converted into R¹—CN. Bromofuran XI can react with alkylzinc reagents in the presence of a palladium catalyst to give XII. Ester XII (or any R¹—CO₂CH₃) is reacted with ammonium hydroxide to give the corresponding amide XIII. Dehydration of the amide is accomplished using POCl₃ in pyridine to give the desired heterocyclic nitrile R¹—CN.

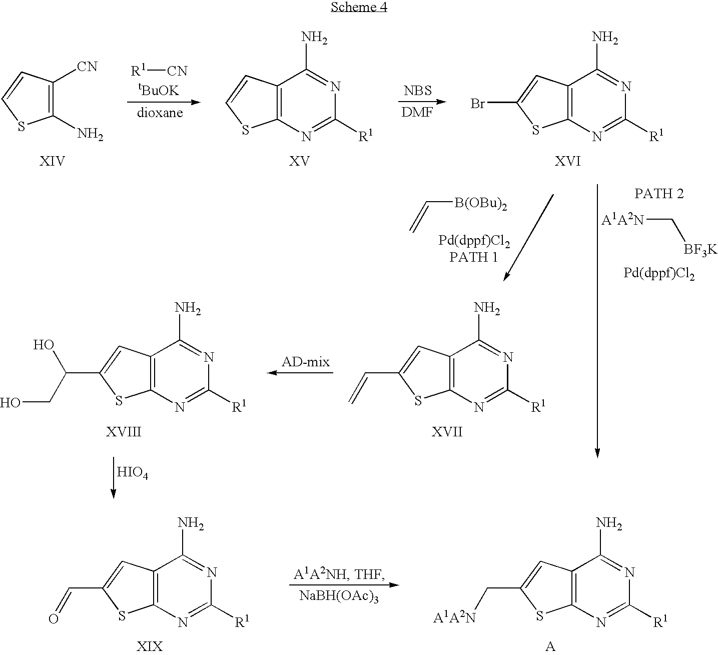

Scheme 5 illustrates the synthetic routes (Paths 1 and 2) leading to compounds of Formula A. Starting with 2-amino-3-cyanothiophene XIV and following path 1 indicated by the arrows, condensation under basic conditions with R¹—CN, where R¹ is as defined in formula A, affords the aminopyrimidine XV. The aminopyrimidine XV is then reacted with N-bromosuccinimide (NBS), which gives the bromothiophene XVI. Following path 1, palladium catalyzed coupling with vinylboronic acid dibutyl ester affords the corresponding vinyl adduct XVII. The olefin present in XVII can be dihydroxylated using AD-mix to give diol XVIII that is then oxidized using periodic acid to afford the aldehyde XIX. Aldehyde XIX can then undergo reductive amination using A¹A²NH, as outlined in scheme 1 to give compounds of the formula A. Alternatively, following path 2, bromothiophene XVI can undergo palladium-catalyzed reactions with aminomethyl potassium trifluoroborates to give compounds of formula A.

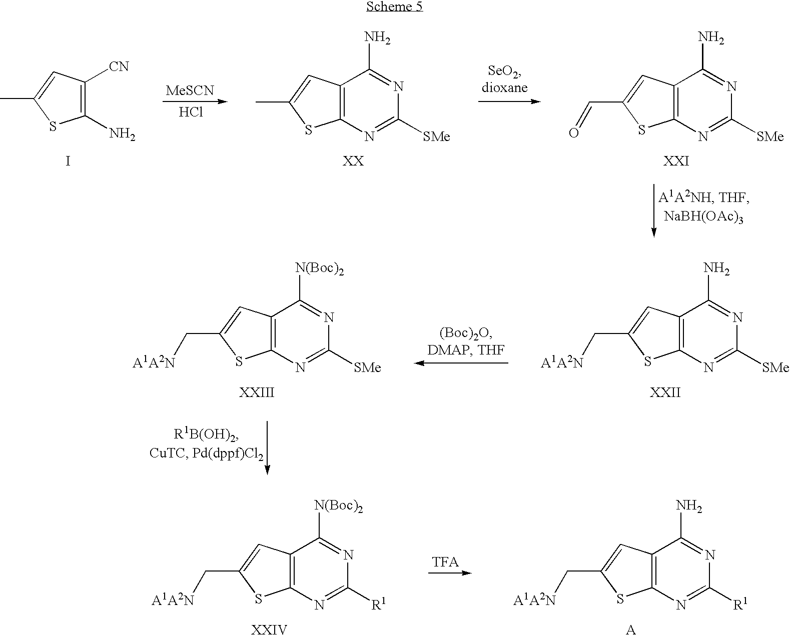

Scheme 5 illustrates the synthetic route leading to compounds of formula A. Starting with 2-amino-5-methyl-thiophene-3-carbonitrile (I) is reacted with methyl thiocyanate in the presence of an acid to form the aminopyrimidine XX. Aminopyrimidine XX can react with selenium dioxide (SeO₂) to give the corresponding aldehyde XXI that can then undergo reductive amination using A¹A²NH, where A¹ and A² are as defined in formula A, to give compound XXII. The aminopyrimidine XXII is reacted with (Boc)₂O in the presence of DMAP to give the corresponding protected amine XXIII. Palladium-catalyzed cross-coupling of the thiomethyl ether functionality can be accomplished with a variety of boronic acids R¹—B(OH)₂ in the presence of copper (I) thiophene-2-carboxylate (CuTC), where R¹ is as defined in formula A, to give the corresponding substituted pyrimidine XXIV. Finally, deprotection using TFA affords compounds of the formula A.

### EXAMPLES

### Example 1

### 2-(5-Chloro-furan-2-yl)-6-(3,3-difluoro-piperidin-1-ylmethyl)-thieno[2,3-d]pyrimidin-4-ylamine

### Example 1

### Step a

### 2-furan-2-yl-6-methyl-thieno[2,3-d]pyrimidin-4-ylamine

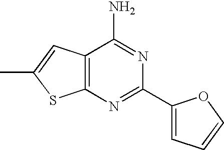

Solid potassium-tert-butoxide (325 mg, 2.9 mmol) was added to a dioxane solution (7 mL) of 2-amino-5-methyl-thiophene-3-carbonitrile (2.0 g, 14.5 mmol) and 2-furonitrile (1.3 g, 14.5 mmol). The resulting mixture was heated at 130° C. for 10 minutes. The dark slurry was cooled to room temperature, diluted with THF, and dry packed onto silica gel. The material was the purified via column chromatography to give 1.6 g of the title compound.

### Example 1

### Step b

### 2-(5-Chloro-furan-2-yl)-6-methyl-thieno[2,3-d]pyrimidin-4-ylamine

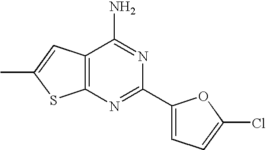

Solid NCS (916 mg, 6.9 mmol) was added to a DMF solution (25 mL) of 2-furan-2-yl-6-methyl-thieno[2,3-d]pyrimidin-4-ylamine (1.4 g, 6.2 mmol) and the mixture was heated to 50° C. After 16 h the mixture was cooled to rt and diluted with water. The precipitated solid was filtered and dried in vacuo to give 1.2 g of the title compound that was used without further purification.

### Example 1

### Step c

### [2-(5-Chloro-furan-2-yl)-6-methyl-thieno[2,3-d]pyrimidin-4-yl]-bis-carbamic acid tert-butyl ester

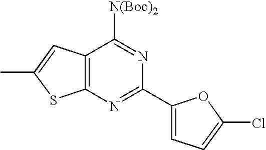

Solid DMAP (29 mg, 0.2 mmol) was added to a THF solution (12 mL) of (Boc)₂O (1.3 g, 5.9 mmol) and 2-(5-Chloro-furan-2-yl)-6-methyl-thieno[2,3-d]pyrimidin-4-ylamine (630 mg, 2.4 mmol). After 6 h the mixture was diluted with EtOAc and the organic layer was washed with water and brine, dried (Na₂SO₄), concentrated and purified via column chromatography to give 928 mg of the title compound.

### Example 1

### Step d

### [6-Bromomethyl-2-(5-chloro-furan-2-yl)-thieno[2,3-d]pyrimidin-4-yl]-bis-carbamic acid tert-butyl ester

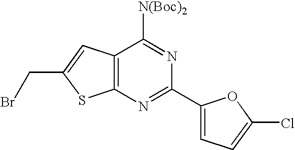

Solid benzoyl peroxide (34 mg, 0.1 mmol) was added to a benzene solution (10 mL) of DBDMH (314 mg, 1.1 mmol) and [2-(5-Chloro-furan-2-yl)-6-methyl-thieno[2,3-d]pyrimidin-4-yl]-bis-carbamic acid tert-butyl ester (928 mg, 2.0 mmol) and the resulting mixture was heated to reflux. After 14 h the mixture was cooled to rt, diluted with EtOAc and the organic layer was washed with water and brine, dried (Na₂SO₄), concentrated and purified via column chromatography to give 651 mg of the title compound.

### Example 1

### Step e

### 6-Bromomethyl-2-(5-chloro-furan-2-yl)-thieno[2,3-d]pyrimidin-4-ylamine

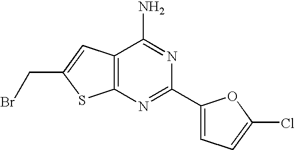

Neat TFA (2 mL) was added to a CH₂Cl₂ solution (8 mL) of [6-Bromomethyl-2-(5-chloro-furan-2-yl)-thieno[2,3-d]pyrimidin-4-yl]-bis-carbamic acid tert-butyl ester (651 mg). After 4 h saturated aqueous NaHCO₃ was added and the aqueous phase was extracted with EtOAc. The combined organics were washed with water and brine, dried (Na₂SO₄), and concentrated to give 369 mg of the title compound that was used without further purification.

### Example 1

### Step f

### 2-(5-Chloro-furan-2-yl)-6-(3,3-difluoro-piperidin-1-ylmethyl)-thieno[2,3-d]pyrimidin-4-ylamine

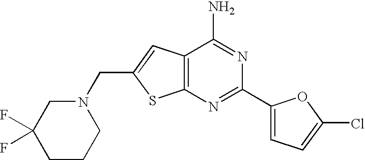

Solid 3,3-difluoro-piperidine hydrochloride (34 mg, 0.22 mmol) was added to a THF solution (1 mL) of diisopropylethyl amine (0.10 mL, 0.56 mmol) and 6-bromomethyl-2-(5-chloro-furan-2-yl)-thieno[2,3-d]pyrimidin-4-ylamine (50 mg, 0.14 mmol) and the mixture was heated to 40° C. After 2 h the mixture was diluted with EtOAc then washed with water and brine, dried (Na₂SO₄), concentrated and purified via column chromatography to give 31 mg of the title compound. ¹H NMR (CHLOROFORM-d, 300 MHz): δ=7.23 (d, J=3.4 Hz, 1H), 6.99 (s, 1H), 6.34 (d, J=3.4 Hz, 1H), 5.31 (br. s., 2H), 3.86 (s, 2H), 2.75 (t, J=11.1 Hz, 2H), 2.57 (t, J=5.1 Hz, 2H), 1.73-1.99 ppm (m, 4H); MS m/e 385 (M+H).

### Example 2

### 2-(5-Chloro-furan-2-yl)-6-(4-trifluoromethyl-piperidin-1-ylmethyl)-thieno[2,3-d]pyrimidin-4-ylamine

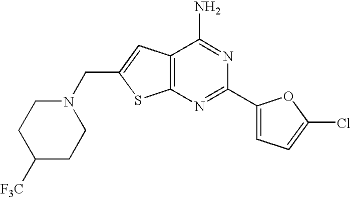

The title compound was prepared using 4-trifluoromethyl-piperidine hydrochloride in place of 3,3-difluoro-piperidine hydrochloride as described in Example 1. ¹H NMR (CHLOROFORM-d, 300 MHz): δ=7.23 (d, J=3.4 Hz, 1H), 6.96 (s, 1H), 6.34 (d, J=3.4 Hz, 1H), 5.28 (s, 2H), 3.75 (s, 2H), 3.02-3.10 (m, 2H), 1.98-2.11 (m, 3H), 1.80-1.91 (m, 2H), 1.61-1.75 ppm (m, 2H); MS m/e 417 (M+H).

### Example 3

### 2-(5-Chloro-furan-2-yl)-6-cyclopropylaminomethyl-thieno[2,3-d]pyrimidin-4-ylamine

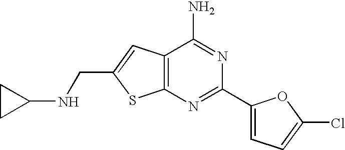

The title compound was prepared using cyclopropylamine in place of 3,3-difluoro-piperidine hydrochloride as described in Example 1. ¹H NMR (CHLOROFORM-d, 300 MHz): δ=7.17-7.24 (m, 1H), 6.97 (s, 1H), 6.33 (d, J=3.4 Hz, 1H), 5.31 (br. s., 2H), 4.09 (s, 2H), 2.17-2.30 (m, 1H), 1.58 (br. s., 1H), 0.37-0.54 ppm (m, 4H); MS m/e 321 (M+H).

### Example 4

### 2-(5-Chloro-furan-2-yl)-6-(3-fluoro-pyrrolidin-1-ylmethyl)-thieno[2,3-d]pyrimidin-4-ylamine

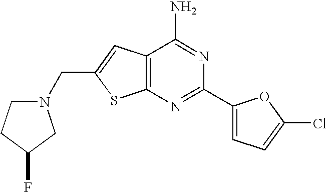

The title compound was prepared using (S)-3-fluoro-pyrrolidine hydrochloride in place of 3,3-difluoro-piperidine hydrochloride as described in Example 1. ¹H NMR (CHLOROFORM-d, 300 MHz): δ=7.22 (d, J=3.4 Hz, 1H), 6.98 (s, 1H), 6.33 (d, J=3.4 Hz, 1H), 5.33 (br. s., 2H), 5.04-5.16 (m, 1H), 3.93 (s, 2H), 2.82-2.99 (m, 3H), 2.57-2.69 (m, 1H), 1.99-2.31 ppm (m, 2H); MS m/e 353 (M+H).

### Example 5

### 2-(5-Chloro-furan-2-yl)-6-morpholin-4-ylmethyl-thieno[2,3-d]pyrimidin-4-ylamine

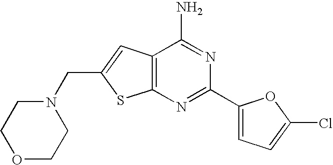

The title compound was prepared using morpholine in place of 3,3-difluoro-piperidine hydrochloride as described in Example 1. ¹H NMR (CHLOROFORM-d, 300 MHz): δ=7.23 (d, J=3.8 Hz, 1H), 6.97 (s, 1H), 6.34 (d, J=3.8 Hz, 1H), 5.39 (br. s., 2H), 3.68-3.80 (m, 6H), 2.46-2.61 ppm (m, 4H); MS m/e 351 (M+H).

### Example 6

### 2-(5-Chloro-furan-2-yl)-6-(3,3-difluoro-pyrrolidin-1-ylmethyl)-thieno[2,3-d]pyrimidin-4-ylamine

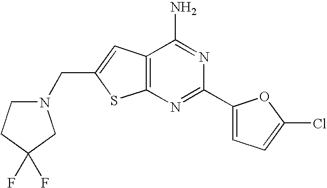

The title compound was prepared using 3,3-difluoro-pyrrolidine hydrochloride in place of 3,3-difluoro-piperidine hydrochloride as described in Example 1. ¹H NMR (CHLOROFORM-d, 300 MHz): δ=7.22-7.26 (m, 1H), 7.00 (s, 1H), 6.34 (d, J=3.4 Hz, 1H), 5.41 (br. s., 2H), 3.90 (s, 2H), 3.01 (t, J=13.2 Hz, 2H), 2.86 (t, J=7.0 Hz, 2H), 2.33 ppm (tt, J=14.4, 7.1 Hz, 2H); MS m/e 371 (M+H).

### Example 7

### 2-(5-Bromo-furan-2-yl)-6-morpholin-4-ylmethyl-thieno[2,3-d]pyrimidin-4-ylamine

### Example 7

### Step a

### 5-Bromo-furan-2-carbonitrile

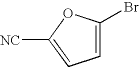

Neat POCl₃ (0.69 mL, 7.4 mmol) was added to a pyridine solution (13 mL) of 5-bromo-furan-2-carboxylic acid amide (1.0 g, 5.3 mmol). After 2 h the mixture was cooled to 0° C. and taken to pH 4.5 with concentrated aqueous HCl. The aqueous mixture was extracted with Et₂O and the combined extracts were washed with brine, dried (Na₂SO₄), concentrated and used without further purification to give 900 mg of the title compound.

### Example 7

### Step b

### 2-(5-Chloro-furan-2-yl)-6-(3,3-difluoro-pyrrolidin-1-ylmethyl)-thieno[2,3-d]pyrimidin-4-ylamine

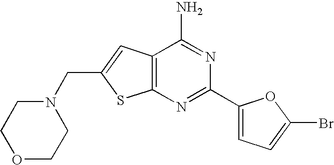

The title compound was prepared using 5-bromo-furan-2-carbonitrile and morpholine in place of 2-furonitrile and 3,3-difluoro-piperidine hydrochloride, respectively, as described in Example 1. ¹H NMR (CHLOROFORM-d, 400 MHz): δ=7.20 (d, J=3.4 Hz, 1H), 6.97 (s, 1H), 6.48 (d, J=3.4 Hz, 1H), 5.40 (br. s, 2H), 3.61-3.86 (m, 6H), 2.40-2.65 ppm (m, 4H); MS m/e 396 (M+H).

### Example 8

### 2-(5-Ethyl-furan-2-yl)-6-morpholin-4-ylmethyl-thieno[2,3-d]pyrimidin-4-ylamine

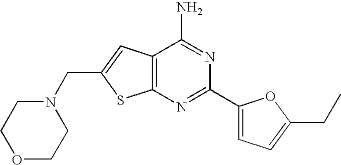

A 1 M THF solution of Et₂Zn (0.6 mL, 0.60 mmol) was added to a THF solution (1.5 mL) of Pd(dppf)Cl₂ (10 mg, 0.01 mmol) and 2-(5-bromo-furan-2-yl)-6-morpholin-4-ylmethyl-thieno[2,3-d]pyrimidin-4-ylamine (60 mg, 0.15 mmol) and the mixture was refluxed. After 4 h the mixture was cooled and carefully diluted with EtOAc and water. The aqueous phase was extracted with EtOAc and the combined organics were washed with water and brine, dried (Na₂SO₄), and dry packed onto silica gel. Column chromatography gave 33 mg of the title compound. ¹H NMR (CHLOROFORM-d, 300 MHz): δ=7.19 (d, J=3.4 Hz, 1H), 6.95 (s, 1H), 6.17 (d, J=3.4 Hz, 1H), 5.32 (s, 2H), 3.68-3.77 (m, 6H), 2.81 (q, J=7.5 Hz, 2H), 2.44-2.58 (m, 4H), 1.25-1.34 ppm (m, 3H); MS m/e 345 (M+H).

### Example 9

### 6-(2,6-Dimethyl-piperidin-1-ylmethyl)-2-(4-methyl-thiazol-2-yl)-thieno[2,3-d]pyrimidin-4-ylamine

### Example 9

### Step a

### 2-(4-Methyl-thiazol-2-yl)-thieno[2,3-d]pyrimidin-4-ylamine

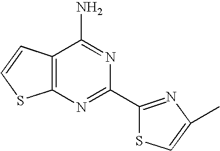

The title compound was prepared using 4-methyl-thiazole-2-carbonitrile and 2-amino-3-cyanothiophene in place of 2-furonitrile and 2-amino-5-methyl-thiophene-3-carbonitrile, respectively, as described in Example 1.

### Example 9

### Step b

### 6-Bromo-2-(4-methyl-thiazol-2-yl)-thieno[2,3-d]pyrimidin-4-ylamine

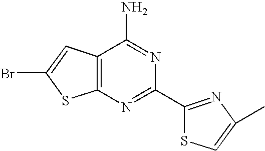

### Example 9

### Step c

### 2-(4-Methyl-thiazol-2-yl)-6-vinyl-thieno[2,3-d]pyrimidin-4-ylamine

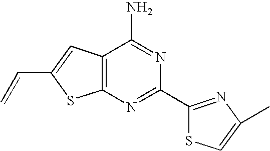

Neat vinylboronic acid dibutyl ester (1.0 mL, 4.7 mmol) was added to a dioxane (20 mL)/water (5 mL) solution of 6-Bromo-2-(4-methyl-thiazol-2-yl)-thieno[2,3-d]pyrimidin-4-ylamine (775 mg, 2.4 mmol), Pd(dppf)Cl₂ (196 mg, 0.2 mmol), and K₂CO₃ (650 mg, 4.7 mmol) and the mixture was heated to 80° C. After 3 h the mixture was cooled and diluted with EtOAc. The organic phase was washed with water and brine, dried (Na₂SO₄) and dry packed onto silica gel. Column chromatography gave 460 mg of the title compound.

### Example 9

### Step d

### 1-[4-Amino-2-(4-methyl-thiazol-2-yl)-thieno[2,3-d]pyrimidin-6-yl]-ethane-1,2-diol

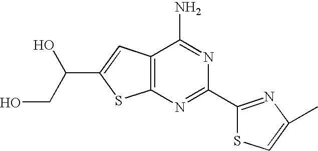

Solid MeSO₂NH₂ (162 mg, 1.7 mmol) was added to a t-BuOH (8 mL)/water (8 mL) solution of AD mix-α (2.4 g). After 15 min the resulting mixture was added to an acetone suspension (8 mL) of 2-(4-methyl-thiazol-2-yl)-6-vinyl-thieno[2,3-d]pyrimidin-4-ylamine (460 mg, 1.7 mmol) and the mixture was stirred vigorously. After 18 h sodium sulfite (2.5 g) was added and the mixture was stirred for an additional 30 minutes. The mixture was extracted with EtOAc and the combined extracts were washed with water and brine, dried (Na₂SO₄), and concentrated to give 350 mg of the title compound that was used without further purification.

### Example 9

### Step e

### 4-Amino-2-(4-methyl-thiazol-2-yl)-thieno[2,3-d]pyrimidine-6-carbaldehyde

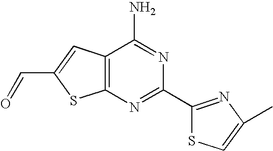

Solid HIO₄ (775 mg, 3.4 mmol) was added to a THF solution (20 mL) of 1-[4-amino-2-(4-methyl-thiazol-2-yl)-thieno[2,3-d]pyrimidin-6-yl]-ethane-1,2-diol (350 mg, 1.1 mmol). After 2 h saturated aqueous NaHCO₃ was added and the aqueous phase was extracted with EtOAc. The combined extracts were washed with water and brine, dried (Na₂SO₄), and dry packed onto silica gel. Column chromatography gave 113 mg of the title compound.

### Example 9

### Step f

### 6-(2,6-Dimethyl-piperidin-1-ylmethyl)-2-(4-methyl-thiazol-2-yl)-thieno[2,3-d]pyrimidin-4-ylamine

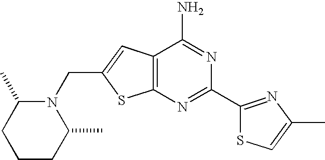

Solid NaBH(OAc)₃ (45 mg, 0.21 mmol) was added to a THF solution (2 mL) of 4-amino-2-(4-methyl-thiazol-2-yl)-thieno[2,3-d]pyrimidine-6-carbaldehyde (40 mg, 0.14 mmol) and cis-2,6-dimethyl-piperidine (58 μL, 0.43 mmol) and the mixture was heated to 45° C. After 16 h the mixture was cooled, diluted with EtOAc, washed with saturated aqueous NaHCO₃, water and brine, dried (Na₂SO₄), and dry packed onto silica gel. Column chromatography gave 15 mg of the title compound. ¹H NMR (Acetone, 300 MHz): δ=7.29 (s, 1H), 7.13 (s, 1H), 6.87 (br. s., 2H), 3.96 (s, 2H), 2.43 (br. s., 2H), 2.34 (s, 3H), 1.38-1.58 (m, 2H), 1.10-1.23 (m, 4H), 1.02 ppm (d, J=6.4 Hz, 6H); MS m/e 374 (M+H).

### Example 10

### 6-(2,6-Dimethyl-piperidin-1-ylmethyl)-2-(5-isopropyl-furan-2-yl)-thieno[2,3-d]pyrimidin-4-ylamine

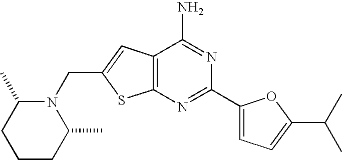

The title compound was prepared using i-PrZnBr and 2-(5-bromo-furan-2-yl)-6-(2,6-dimethyl-piperidin-1-ylmethyl)-thieno[2,3-d]pyrimidin-4-ylamine in place of Et₂Zn and 2-(5-bromo-furan-2-yl)-6-morpholin-4-ylmethyl-thieno[2,3-d]pyrimidin-4-ylamine, respectively, as described in Example 8. ¹H NMR (CHLOROFORM-d, 300 MHz): δ=7.17 (d, J=3.4 Hz, 1H), 6.95 (s, 1H), 6.14 (d, J=3.0 Hz, 1H), 5.34 (br. s., 2H), 4.13 (s, 2H), 3.12 (quin, J=6.9 Hz, 1H), 2.57 (br. s., 2H), 1.51-1.80 (m, 6H), 1.32 (d, J=6.8 Hz, 6H), 1.20 ppm (d, J=6.0 Hz, 6H); MS m/e 385 (M+H).

### Example 11

### 6-(2,6-Dimethyl-piperidin-1-ylmethyl)-2-(5-ethyl-furan-2-yl)-thieno[2,3-d]pyrimidin-4-ylamine

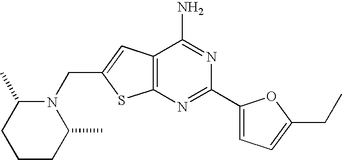

The title compound was prepared using 2-(5-bromo-furan-2-yl)-6-(2,6-dimethyl-piperidin-1-ylmethyl)-thieno[2,3-d]pyrimidin-4-ylamine in place of 2-(5-bromo-furan-2-yl)-6-morpholin-4-ylmethyl-thieno[2,3-d]pyrimidin-4-ylamine as described in Example 8. ¹H NMR (CHLOROFORM-d, 300 MHz): δ=7.18 (d, J=3.4 Hz, 1H), 6.95 (s, 1H), 6.16 (d, J=3.4 Hz, 1H), 5.34 (br. s., 2H), 4.13 (s, 2H), 2.81 (q, J=7.4 Hz, 2H), 2.57 (br. s., 2H), 1.78 (br. s., 4H), 1.51-1.70 (m, 2H), 1.23-1.35 (m, 3H), 1.21 ppm (d, J=6.0 Hz, 6H); MS m/e 371 (M+H).

### Example 12

### 2-(5-Cyclopropyl-furan-2-yl)-6-(2,6-dimethyl-piperidin-1-ylmethyl)-thieno[2,3-d]pyrimidin-4-ylamine

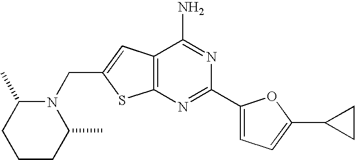

Solid cyclopropylboronic acid (31 mg, 0.36 mmol) was added to a toluene (1 mL)/water (0.05 mL) suspension of 2-(5-bromo-furan-2-yl)-6-(2,6-dimethyl-piperidin-1-ylmethyl)-thieno[2,3-d]pyrimidin-4-ylamine (60 mg, 0.14 mmol), Pd(OAc)₂ (2 mg, 0.01 mmol), P(Cy)₃ (5 mg, 0.02 mmol) and K₃PO₄ (104 mg, 0.49 mmol) and the mixture was heated to 100° C. After 4 h the mixture was cooled, diluted with EtOAc, washed with water and brine, dried (Na₂SO₄) and dry packed onto silica gel. Column chromatography gave 30 mg of the title compound. ¹H NMR (CHLOROFORM-d, 300 MHz): δ=7.15 (d, J=3.4 Hz, 1H), 6.87-6.99 (m, 1H), 6.03 (d, J=3.0 Hz, 1H), 5.27 (s, 2H), 4.12 (s, 2H), 2.56 (br. s., 2H), 2.00-2.11 (m, 1H), 1.58-1.71 (m, 2H), 1.23-1.41 (m, 4H), 1.21 (s, 3H), 1.19 (s, 3H), 0.89-0.99 (m, 2H), 0.79-0.88 ppm (m, 2H); MS m/e 383 (M+H).

### Example 13

### 2-(5-tert-Butyl-thiophen-2-yl)-6-(2,6-dimethyl-piperidin-1-ylmethyl)-thieno[2,3-d]pyrimidin-4-ylamine

### Example 13

### Step a

### 2-(5-tert-Butyl-thiophen-2-yl)-6-methyl-thieno[2,3-d]pyrimidin-4-ylamine

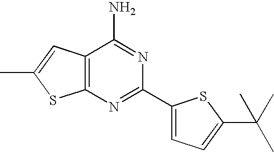

The title compound was prepared using 5-tert-butyl-thiophene-2-carbonitrile in place of 2-furonitrile as described in Example 1.

### Example 13

### Step b

### 4-Amino-2-(5-tert-butyl-thiophen-2-yl)-thieno[2,3-d]pyrimidine-6-carbaldehyde

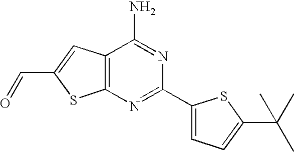

Solid SeO₂ (1.3 g, 11.6 mmol) was added to a dioxane (20 mL)/water (0.2 mL) suspension of 2-(5-tert-butyl-thiophen-2-yl)-6-methyl-thieno[2,3-d]pyrimidin-4-ylamine (885 mg, 2.9 mmol) and the mixture was heated to 100° C. After 20 h the mixture was filtered hot and diluted with EtOAc. The organic phase was washed with water and brine, dried (Na₂SO₄) and dry packed onto silica gel. Column chromatography gave 521 mg of the title compound.

### Example 13

### Step c

### 2-(5-tert-Butyl-thiophen-2-yl)-6-(2,6-dimethyl-piperidin-1-ylmethyl)-thieno[2,3-d]pyrimidin-4-ylamine

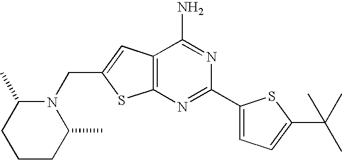

Solid NaBH(OAc)₃ (124 mg, 0.59 mmol) was added to a THF solution (3 mL) of cis-2,6-dimethyl-piperidine (0.16 mL, 1.18 mmol) and 4-amino-2-(5-tert-butyl-thiophen-2-yl)-thieno[2,3-d]pyrimidine-6-carbaldehyde (125 mg, 0.39 mmol) and the mixture was heated to 45° C. After 16 h the mixture was cooled and diluted with EtOAc. The organic phase was washed with water and brine, dried (Na₂SO₄) and dry packed onto silica gel. Column chromatography gave 60 mg of the title compound. ¹H NMR (CHLOROFORM-d, 300 MHz): δ=7.75 (d, J=3.8 Hz, 1H), 6.93 (s, 1H), 6.85 (d, J=3.8 Hz, 1H), 5.31 (s, 2H), 4.12 (s, 2H), 2.48-2.64 (m, 2H), 1.53-1.70 (m, 2H), 1.42 (s, 9H), 1.22-1.37 (m, 4H), 1.21 (s, 3H), 1.19 ppm (s, 3H); MS m/e 415 (M+H).

### Example 14

### 2-(5-tert-Butyl-thiophen-2-yl)-6-morpholin-4-ylmethyl-thieno[2,3-d]pyrimidin-4-ylamine

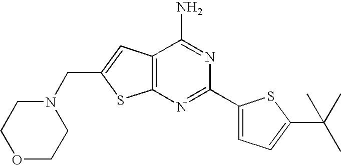

The title compound was prepared using morpholine in place of cis-2,6-dimethyl-piperidine as described in Example 13. ¹H NMR (CHLOROFORM-d, 300 MHz): δ=7.76 (d, J=3.8 Hz, 1H), 6.94 (s, 1H), 6.85 (d, J=3.8 Hz, 1H), 5.18 (s, 2H), 3.63-3.81 (m, 6H), 2.42-2.62 (m, 4H), 1.42 ppm (s, 9H); MS m/e 389 (M+H).

### Example 15

### 2-(4-Methyl-thiazol-2-yl)-6-morpholin-4-ylmethyl-thieno[2,3-d]pyrimidin-4-ylamine

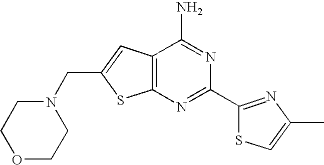

The title compound was prepared using morpholine in place of cis-2,6-dimethyl-piperidine as described in Example 9. ¹H NMR (Acetone, 300 MHz): δ=7.31 (s, 1H), 7.13 (s, 1H), 6.88 (br. s., 2H), 3.65 (s, 2H), 3.47-3.56 (m, 4H), 2.35-2.40 (m, 4H), 2.34 ppm (s, 3H); MS m/e 348 (M+H).

### Example 16

### 2-Isoxazol-3-yl-6-morpholin-4-ylmethyl-thieno[2,3-d]pyrimidin-4-ylamine

### Example 16

### Step a

### Isoxazole-3-carboxylic acid amide

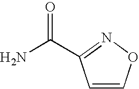

Solid NaH (60% wt in oil) (425 mg, 10.6 mmol) was added to a THF solution (50 mL) of isoxazole-3-carboxylic acid (1.0 g, 8.8 mmol). After 15 min neat ethylchloroformate (1.0 mL, 10.6 mmol) was added. After 45 min a 7 N ammonia solution in MeOH (5.0 mL, 35 mmol) was added. After 30 min the mixture was diluted with EtOAc washed with water and brine, dried (Na₂SO₄) and dry packed onto silica gel. Column chromatography gave 600 mg of the title compound.

### Example 16

### Step b

### Isoxazole-3-carbonitrile

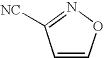

The title compound was prepared using isoxazole-3-carboxylic acid amide in place of 5-bromo-furan-2-carboxylic acid amide as described in example X.

### Example 16

### Step c

### 2-Isoxazol-3-yl-6-morpholin-4-ylmethyl-thieno[2,3-d]pyrimidin-4-ylamine

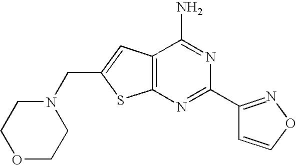

The title compound was prepared using morpholine and isoxazole-3-carbonitrile in place of cis-2,6-dimethyl-piperidine and 5-tert-butyl-thiophene-2-carbonitrile, respectively, as described in Example 13. ¹H NMR (Acetone, 300 MHz): δ=8.68 (d, J=1.5 Hz, 1H), 7.33 (s, 1H), 6.83-6.91 (m, 3H), 3.66 (s, 2H), 3.46-3.56 (m, 4H), 2.31-2.43 ppm (m, 4H); MS m/e 318 (M+H).

### Example 17

### 3-[4-Amino-6-(2,6-dimethyl-morpholin-4-ylmethyl)-thieno[2,3-d]pyrimidin-2-yl]-benzonitrile

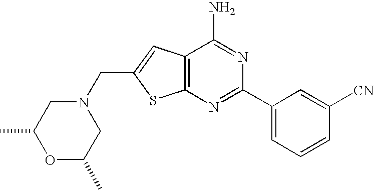

The title compound was prepared using cis-2,6-dimethyl-morpholine and 1,3-dicyanobenzene in place of cis-2,6-dimethyl-piperidine and 5-tert-butyl-thiophene-2-carbonitrile, respectively, as described in Example 13. ¹H NMR (Acetone, 300 MHz): δ=8.54-8.65 (m, 2H), 7.72 (d, J=7.9 Hz, 1H), 7.57 (t, J=8.1 Hz, 1H), 7.30 (s, 1H), 6.84 (br. s., 2H), 3.64 (s, 2H), 3.42-3.58 (m, 2H), 2.62-2.77 (m, 2H), 1.63 (t, J=10.7 Hz, 2H), 0.95 (s, 3H), 0.93 ppm (s, 3H); MS m/e 380 (M+H).

### Example 18

### 6-(2,6-Dimethyl-piperidin-1-ylmethyl)-2-isoxazol-3-yl-thieno[2,3-d]pyrimidin-4-ylamine

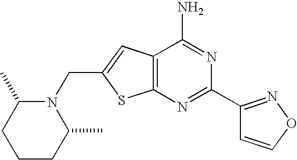

The title compound was prepared using isoxazole-3-carbonitrile in place of 5-tert-butyl-thiophene-2-carbonitrile as described in Example 13. ¹H NMR (Acetone, 300 MHz): δ=8.67 (d, J=1.9 Hz, 1H), 7.30 (s, 1H), 6.88 (d, J=1.5 Hz, 1H), 6.82 (br. s., 2H), 3.97 (s, 2H), 2.33-2.51 (m, 2H), 1.42-1.56 (m, 2H), 1.10-1.26 (m, 4H), 1.03 ppm (d, J=6.0 Hz, 6H); MS m/e 344 (M+H).

### Example 19

### 2-(5-Bromo-furan-2-yl)-6-(2,6-dimethyl-piperidin-1-ylmethyl)-thieno[2,3-d]pyrimidin-4-ylamine

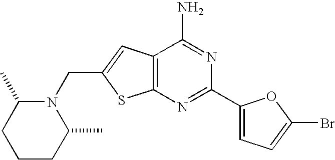

The title compound was prepared using 5-bromo-furan-2-carbonitrile and cis-2,6-dimethyl-piperidine in place of 2-furonitrile and 3,3-difluoro-piperidine hydrochloride, respectively, as described in Example 1. ¹H NMR (CHLOROFORM-d, 400 MHz): δ=7.19 (d, J=3.4 Hz, 1H), 6.95 (s, 1H), 6.48 (d, J=3.7 Hz, 1H), 5.44 (br. s., 2H), 4.11 (s, 2H), 2.45-2.66 (m, 2H), 1.55-1.70 (m, 2H), 1.26-1.39 (m, 4H), 1.19 ppm (d, J=6.1 Hz, 6H); MS m/e 422 (M+H).

### Example 20

### 3-[4-Amino-6-(2-phenyl-pyrrolidin-1-ylmethyl)-thieno[2,3-d]pyrimidin-2-yl]-benzonitrile

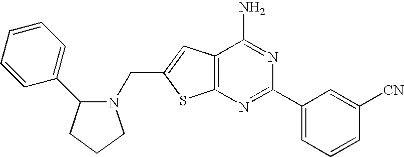

The title compound was prepared using 2-phenyl-pyrrolidine and 1,3-dicyanobenzene in place of cis-2,6-dimethyl-piperidine and 5-tert-butyl-thiophene-2-carbonitrile, respectively, as described in Example 13. ¹H NMR (CHLOROFORM-d, 400 MHz): δ=8.76 (t, J=1.5 Hz, 1H), 8.67 (dt, J=8.1, 1.3 Hz, 1H), 7.69 (dt, J=7.6, 1.5 Hz, 1H), 7.55 (t, J=7.8 Hz, 1H), 7.43-7.50 (m, 2H), 7.31-7.40 (m, 2H), 7.19-7.30 (m, 1H), 6.91 (s, 1H), 5.23 (s, 2H), 3.99 (dd, J=14.4, 1.5 Hz, 1H), 3.41-3.53 (m, 2H), 3.24-3.35 (m, 1H), 2.35 (q, J=8.5 Hz, 1H), 2.14-2.29 (m, 1H), 1.89-2.02 (m, 1H), 1.69-1.89 ppm (m, 2H); MS m/e 412 (M+H).

### Example 21

### 2-(5-Isopropyl-furan-2-yl)-6-morpholin-4-ylmethyl-thieno[2,3-d]pyrimidin-4-ylamine

### Example 21

### Step a

### 5-Isopropyl-furan-2-carboxylic acid methyl ester

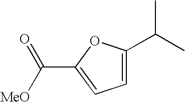

A 0.5 M THF solution (7.3 mL, 3.6 mmol) of isopropylzinc bromide was added to a THF solution (2 mL) of 5-bromo-furan-2-carboxylic acid methyl ester (250 mg, 1.2 mmol) and Pd(dppf)Cl₂ (98 mg, 0.1 mmol) and the resulting mixture was heated to 70° C. After 15 h the mixture was cooled, water was added and the aqueous phase was extracted with EtOAc. The combined organic extracts were washed with water and brine, dried (Na₂SO₄), concentrated and purified via column chromatography to give 150 mg of 5-isopropyl-furan-2-carboxylic acid methyl ester. Steps b and c of Example 14 were followed to access the desired carbonitrile.

### Example 21

### Step b

### 5-Isopropyl-furan-2-carboxylic acid amide

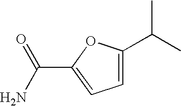

5-isopropyl-furan-2-carboxylic acid methyl ester (150 mg, 3.9 mmol) was suspended in concentrated NH₄OH (5 mL) and stirred vigorously. After 16 h the mixture was diluted with water and the aqueous phase was extracted with EtOAc. The combined organic extracts were washed with water and brine, dried (Na₂SO₄), concentrated and used without further purification to give 110 mg of the title compound.

### Example 21

### Step c

### 5-Isopropyl-furan-2-carbonitrile

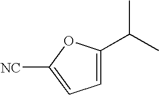

The title compound was prepared using 5-isopropyl-furan-2-carboxylic acid amide in place of isoxazole-3-carboxylic acid as described in example X

### Example 21

### Step d

### 2-(5-Isopropyl-furan-2-yl)-thieno[2,3-d]pyrimidin-4-ylamine

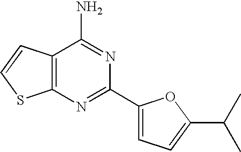

The title compound was prepared using 4-methyl-thiazole-2-carbonitrile and 2-amino-3-cyanothiophene in place of 2-furonitrile and 2-amino-5-methyl-thiophene-3-carbonitrile, respectively, as described in Example 1.

### Example 21

### Step e

### 6-Bromo-2-(5-isopropyl-furan-2-yl)-thieno[2,3-d]pyrimidin-4-ylamine

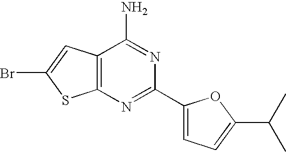

The title compound was prepared using 2-(5-isopropyl-furan-2-yl)-thieno[2,3-d]pyrimidin-4-ylamine in place of 2-(4-methyl-thiazol-2-yl)-thieno[2,3-d]pyrimidin-4-ylamine as described in example 9

### Example 21

### Step f

### Potassium trifluoro[(morpholin-1-yl)methyl]borate

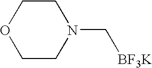

Solid potassium bromomethyltrifluoroborate (200 mg, 1.0 mmol) was added to neat morpholine (4 mL) and the mixture was heated to 80° C. After 30 min the mixture was concentrated in vacuo. The resulting solid was dissolved in an acetone solution (30 mL) of K₂CO₃ (138 mg, 1.0 mmol) and stirred. After 30 min the insoluble salts were filtered off and the filtrate was concentrated in vacuo to give 103 mg of the title compound that was used without further purification.

### Example 21

### Step g

### 2-(5-Isopropyl-furan-2-yl)-6-morpholin-4-ylmethyl-thieno[2,3-d]pyrimidin-4-ylamine

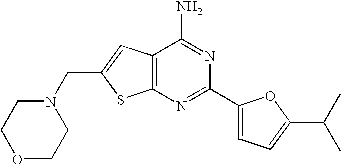

Solid 6-bromo-2-(5-isopropyl-furan-2-yl)-thieno[2,3-d]pyrimidin-4-ylamine (30 mg, 0.09 mmol) was added to THF (1 mL)/water (0.1 mL) solution of potassium trifluoro[(morpholin-1-yl)methyl]borate (103 mg, 0.50 mmol), Pd(OAc)₂ (1 mg, 0.004 mmol), Xphos (4 mg, 0.009 mmol), and Cs₂CO₃ (88 mg, 0.27 mmol) and the resulting mixture was refluxed. After 18 h the mixture was cooled, diluted with EtOAc, washed with water and brine, dried (Na₂SO₄) and dry packed onto silica gel. Column chromatography gave 13 mg of the title compound. ¹H NMR (CHLOROFORM-d, 400 MHz): δ=7.18 (d, J=3.4 Hz, 1H), 6.95 (s, 1H), 6.15 (d, J=3.4 Hz, 1H), 5.26 (br. s, 2H), 3.68-3.79 (m, 6H), 3.07-3.19 (m, 1H), 2.45-2.59 (m, 4H), 1.32 ppm (d, J=7.1 Hz, 6H); MS m/e 359 (M+H).

### Example 22

### 2-(5-Cyclopropyl-furan-2-yl)-6-morpholin-4-ylmethyl-thieno[2,3-d]pyrimidin-4-ylamine

### Example 22

### Step a

### 5-Cyclopropyl-furan-2-carboxylic acid methyl ester

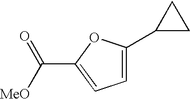

Solid cyclopropylboronic acid (575 mg, 6.7 mmol) was added to a toluene (22 mL)/water (1.1 mL) solution of 5-bromo-furan-2-carboxylic acid methyl ester (980 mg, 4.8 mmol), Pd(OAc)₂ (54 mg, 0.2 mmol), P(Cy)₃ (135 mg, 0.5 mmol), and K₃PO₄ (3.6 g, 16.8 mmol). The resulting mixture was heated to 90° C. After 5 h the mixture was cooled, filtered and extracted with EtOAc. The combined organic extracts were washed with water and brine, dried (Na₂SO₄), concentrated and purified via column chromatography to give 650 mg of 5-cyclopropyl-furan-2-carboxylic acid methyl ester.

### Example 22

### Step b

### 5-Cyclopropyl-furan-2-carboxylic acid amide

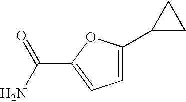

5-cyclopropyl-furan-2-carboxylic acid methyl ester (650 mg, 3.9 mmol) was suspended in concentrated NH₄OH (20 mL) and stirred vigorously. After 16 h the mixture was diluted with water and the aqueous phase was extracted with EtOAc. The combined organic extracts were washed with water and brine, dried (Na₂SO₄), concentrated and used without further purification to give 550 mg of 5-cyclopropyl-furan-2-carboxylic acid amide.

### Example 22

### Step c

### 5-Cyclopropyl-furan-2-carbonitrile

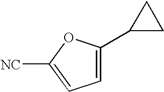

Neat POCl₃ (0.48 mL, 5.1 mmol) was added to a pyridine solution (9 mL) of 5-cyclopropyl-furan-2-carboxylic acid amide (550 mg, 3.6 mmol). After 2 h the mixture was cooled to 0° C. and taken to pH 4.5 with concentrated aqueous HCl. The aqueous mixture was extracted with Et₂O and the combined extracts were washed with brine, dried (Na₂SO₄), concentrated and used without further purification to give 478 mg of 5-cyclopropyl-furan-2-carbonitrile.

### Example 22

### Step d

### 2-(5-Cyclopropyl-furan-2-yl)-6-methyl-thieno[2,3-d]pyrimidin-4-ylamine

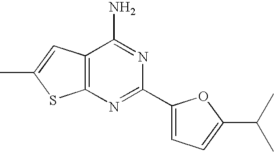

The title compound was prepared using 5-cyclopropyl-furan-2-carbonitrile in place of 2-furonitrile as described in Example 1.

### Example 22

### Step e

### 2-(5-Cyclopropyl-furan-2-yl)-6-morpholin-4-ylmethyl-thieno[2,3-d]pyrimidin-4-ylamine

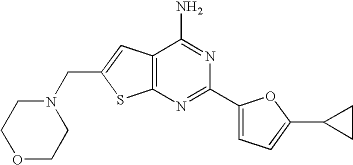

The title compound was prepared using morpholine and 5-cyclopropyl-furan-2-carbonitrile in place of cis-2,6-dimethyl-piperidine and 5-tert-butyl-thiophene-2-carbonitrile, respectively, as described in Example 13. ¹H NMR (CHLOROFORM-d, 300 MHz): δ=7.16 (d, J=3.4 Hz, 1H), 6.95 (s, 1H), 6.03 (d, J=3.4 Hz, 1H), 5.29 (s, 2H), 3.62-3.83 (m, 6H), 2.47-2.56 (m, 4H), 1.99-2.12 (m, 1H), 0.90-1.00 (m, 2H), 0.79-0.89 ppm (m, 2H); MS m/e 357 (M+H).

### Example 23

### 3-[4-Amino-6-(2,6-dimethyl-piperidin-1-ylmethyl)-thieno[2,3-d]pyrimidin-2-yl]-benzonitrile

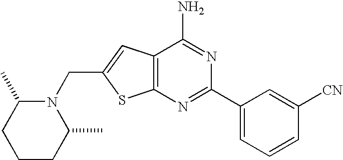

The title compound was prepared using 1,3-dicyanobenzene in place of and 5-tert-butyl-thiophene-2-carbonitrile, respectively, as described in Example 13. ¹H NMR (CHLOROFORM-d, 300 MHz): δ=8.77 (s, 1H), 8.68 (d, J=7.9 Hz, 1H), 7.70 (d, J=7.9 Hz, 1H), 7.56 (t, J=7.7 Hz, 1H), 7.00 (s, 1H), 5.25 (br. s., 2H), 3.74 (d, J=2.3 Hz, 2H), 2.80-3.00 (m, 2H), 1.93-2.15 (m, 2H), 1.57-1.78 (m, 2H), 1.19-1.39 (m, 2H), 0.82-0.97 ppm (m, 6H); MS m/e 378 (M+H).

### Example 24

### 6-(2,6-Dimethyl-piperidin-1-ylmethyl)-2-(5-methyl-furan-2-yl)-thieno[2,3-d]pyrimidin-4-ylamine

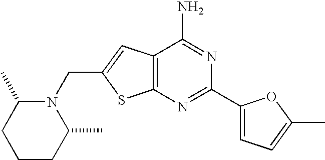

Solid methylboronic acid (34 mg, 0.57 mmol) was added to a dioxane (1.6 mL)/water (0.4 mL) solution of 2-(5-bromo-furan-2-yl)-6-(2,6-dimethyl-piperidin-1-ylmethyl)-thieno[2,3-d]pyrimidin-4-ylamine (60 mg, 0.14 mmol), Pd(dppf)Cl₂ (11 mg, 0.01 mmol), and K₂CO₃ (79 mg, 0.57 mmol) and the mixture was heated to 80° C. After 6 h the mixture was cooled, diluted with EtOAc, washed with water and brine, dried (Na₂SO₄) and dry packed onto silica gel. Column chromatography gave 29 mg of the title compound. ¹H NMR (CHLOROFORM-d, 300 MHz): δ=7.16 (d, J=3.4 Hz, 1H), 6.94 (s, 1H), 6.15 (d, J=2.3 Hz, 1H), 5.27 (br. s, 2H), 4.12 (s, 2H), 2.50-2.64 (m, 2H), 2.45 (s, 3H), 1.24-1.39 (m, 6H), 1.20 ppm (d, J=6.0 Hz, 6H); MS m/e 357 (M+H).

### Example 25

### 6-Morpholin-4-ylmethyl-2-thiazol-2-yl-thieno[2,3-d]pyrimidin-4-ylamine

### Example 25

### Step a

### Thiazole-2-carbonitrile

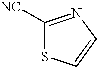

The title compound was prepared using thiazole-2-carboxylic acid methyl ester in place of 5-isopropyl-furan-2-carboxylic acid methyl ester as described in example 21.

### Example 25

### Step b

### 6-Morpholin-4-ylmethyl-2-thiazol-2-yl-thieno[2,3-d]pyrimidin-4-ylamine

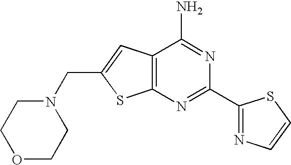

The title compound was prepared using morpholine and thiazole-2-carbonitrile in place of cis-2,6-dimethyl-piperidine and 5-tert-butyl-thiophene-2-carbonitrile, respectively, as described in Example 13. ¹H NMR (CHLOROFORM-d, 400 MHz): δ=7.99 (d, J=3.2 Hz, 1H), 7.48 (d, J=3.2 Hz, 1H), 7.03 (s, 1H), 5.49 (br. s., 2H), 3.66-3.80 (m, 6H), 2.46-2.63 ppm (m, 4H); MS m/e 334 (M+H).

### Example 26

### 6-Morpholin-4-ylmethyl-2-thiophen-2-yl-thieno[2,3-d]pyrimidin-4-ylamine

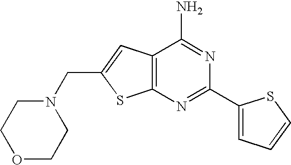

The title compound was prepared using morpholine and thiophene-2-carbonitrile in place of cis-2,6-dimethyl-piperidine and 5-tert-butyl-thiophene-2-carbonitrile, respectively, as described in Example 13. ¹H NMR (CHLOROFORM-d, 300 MHz): δ=7.95 (dd, J=3.7, 1.2 Hz, 1H), 7.41 (dd, J=5.0, 1.2 Hz, 1H), 7.12 (dd, J=5.0, 3.7 Hz, 1H), 6.96 (s, 1H), 5.18 (br. s., 2H), 3.65-3.85 (m, 6H), 2.47-2.65 ppm (m, 4H); MS m/e 333 (M+H).

### Example 27

### 2-(5-Chloro-furan-2-yl)-6-(2-methoxymethyl-pyrrolidin-1-ylmethyl)-thieno[2,3-d]pyrimidin-4-ylamine

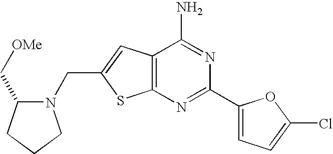

The title compound was prepared using (R)-2-methoxymethyl-pyrrolidine in place of 3,3-difluoro-piperidine hydrochloride as described in Example 1. ¹H NMR (CHLOROFORM-d, 300 MHz): δ=7.22 (d, J=3.4 Hz, 1H), 6.96 (s, 1H), 6.33 (d, J=3.4 Hz, 1H), 5.39 (s, 2H), 4.31 (d, J=14.3 Hz, 1H), 3.78 (d, J=14.3 Hz, 1H), 3.37-3.48 (m, 2H), 3.36 (s, 3H), 3.09 (ddd, J=9.1, 6.5, 3.2 Hz, 1H), 2.73-2.91 (m, 1H), 2.22-2.45 (m, 1H), 1.85-2.01 (m, 1H), 1.53-1.84 ppm (m, 3H); MS m/e 379 (M+H).

### Example 28

### 2-Furan-2-yl-6-pyrrolidin-1-ylmethyl-thieno[2,3-d]pyrimidin-4-ylamine

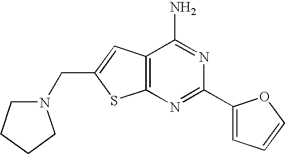

The title compound was prepared using pyrrolidine and 4-amino-2-furan-2-yl-thieno[2,3-d]pyrimidine-6-carbaldehyde in place of cis-2,6-dimethyl-piperidine and 5-tert-butyl-thiophene-2-carbonitrile, respectively, as described in Example 13. ¹H NMR (CHLOROFORM-d, 400 MHz): δ=7.59 (s, 1H), 7.25 (d, J=3.3 Hz, 1H), 7.06 (s, 1H), 6.54 (dd, J=3.3, 1.8 Hz, 1H), 5.44 (br. s., 2H), 3.92 (s, 2H), 2.61-2.74 (m, 4H), 1.85 ppm (dt, J=6.8, 3.3 Hz, 4H); MS m/e 301 (M+H).

### Example 29

### 6-Cyclopropylaminomethyl-2-furan-2-yl-thieno[2,3-d]pyrimidin-4-ylamine

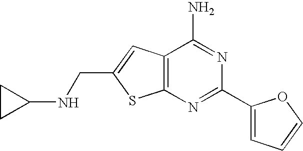

The title compound was prepared using cyclopropylamine and 4-amino-2-furan-2-yl-thieno[2,3-d]pyrimidine-6-carbaldehyde in place of cis-2,6-dimethyl-piperidine and 5-tert-butyl-thiophene-2-carbonitrile, respectively, as described in Example 13. ¹H NMR (CHLOROFORM-d, 300 MHz): δ=7.60 (d, J=0.8 Hz, 1H), 7.25 (s, 1H), 6.99 (s, 1H), 6.55 (dd, J=3.5, 1.8 Hz, 1H), 5.42 (br. s., 2H), 4.09 (s, 2H), 2.15-2.32 (m, 1H), 0.33-0.56 ppm (m, 4H); MS m/e 287 (M+H).

### Example 30

### 6-Pyrrolidin-1-ylmethyl-2-thiophen-2-yl-thieno[2,3-d]pyrimidin-4-ylamine

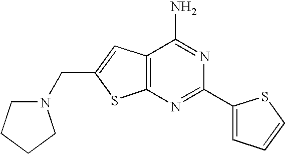

The title compound was prepared using pyrrolidine and 4-amino-2-thiophen-2-yl-thieno[2,3-d]pyrimidine-6-carbaldehyde in place of cis-2,6-dimethyl-piperidine and 5-tert-butyl-thiophene-2-carbonitrile, respectively, as described in Example 13. ¹H NMR (CHLOROFORM-d, 300 MHz): δ=7.95 (dd, J=3.7, 1.2 Hz, 1H), 7.41 (dd, J=5.0, 1.2 Hz, 1H), 7.11 (dd, J=5.0, 3.7 Hz, 2H), 5.33 (br. s., 2H), 3.95 (s, 2H), 2.71 (br. s., 4H), 1.86 ppm (dt, J=6.7, 3.2 Hz, 4H); MS m/e 317 (M+H).

### Example 31

### 6-Diethylaminomethyl-2-furan-2-yl-thieno[2,3-d]pyrimidin-4-ylamine

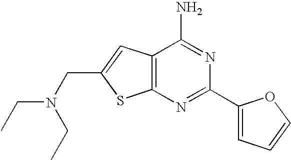

The title compound was prepared using diethylamine and 4-amino-2-furan-2-yl-thieno[2,3-d]pyrimidine-6-carbaldehyde in place of cis-2,6-dimethyl-piperidine and 5-tert-butyl-thiophene-2-carbonitrile, respectively, as described in Example 13. ¹H NMR (CHLOROFORM-d, 300 MHz): δ=7.59 (dd, J=1.7, 0.9 Hz, 1H), 7.25 (dd, J=3.5, 0.8 Hz, 1H), 6.96 (s, 1H), 6.54 (dd, J=3.4, 1.7 Hz, 1H), 5.28 (br. s., 2H), 3.83 (s, 2H), 2.61 (q, J=7.2 Hz, 4H), 1.08 ppm (t, J=7.2 Hz, 6H); MS m/e 303 (M+H).

### Example 32

### 6-Cyclohexylaminomethyl-2-furan-2-yl-thieno[2,3-d]pyrimidin-4-ylamine

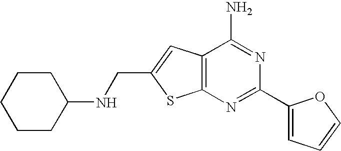

The title compound was prepared using cyclohexylamine and 4-amino-2-furan-2-yl-thieno[2,3-d]pyrimidine-6-carbaldehyde in place of cis-2,6-dimethyl-piperidine and 5-tert-butyl-thiophene-2-carbonitrile, respectively, as described in Example 13. ¹H NMR (CHLOROFORM-d, 300 MHz): δ=7.59 (d, J=0.8 Hz, 1H), 7.24 (d, J=3.4 Hz, 1H), 7.06 (s, 1H), 6.54 (dd, J=3.4, 1.7 Hz, 1H), 5.44 (br. s., 2H), 4.05-4.13 (m, 2H), 3.49 (s, 1H), 2.52-2.66 (m, 1H), 2.06 (s, 2H), 1.73 (br. s., 2H), 1.06-1.35 ppm (m, 6H); MS m/e 329 (M+H).

### Example 33

### 2-Furan-2-yl-6-[(2-methoxy-ethylamino)-methyl]-thieno[2,3-d]pyrimidin-4-ylamine

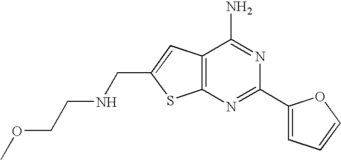

The title compound was prepared using 2-methoxy-ethylamine and 4-amino-2-furan-2-yl-thieno[2,3-d]pyrimidine-6-carbaldehyde in place of cis-2,6-dimethyl-piperidine and 5-tert-butyl-thiophene-2-carbonitrile, respectively, as described in Example 13. ¹H NMR (CHLOROFORM-d, 300 MHz): δ=7.56-7.61 (m, 1H), 7.25 (d, J=3.4 Hz, 1H), 6.98 (s, 1H), 6.54 (dd, J=3.4, 1.7 Hz, 1H), 5.29 (br. s., 2H), 4.07 (s, 2H), 3.50-3.57 (m, 2H), 3.37 (s, 3H), 2.81-2.90 ppm (m, 2H); MS m/e 305 (M+H).

### Example 34

### 2-(5-Methyl-furan-2-yl)-6-piperidin-1-ylmethyl-thieno[2,3-d]pyrimidin-4-ylamine

### Example 34

### Step a

### 4-Amino-2-(5-methyl-furan-2-yl)-thieno[2,3-d]pyrimidine-6-carbaldehyde

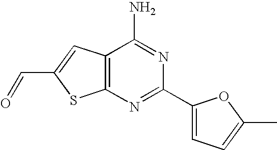

The title compound was prepared using 1-[4-Amino-2-(5-methyl-furan-2-yl)-thieno[2,3-d]pyrimidin-6-yl]-ethane-1,2-diol in place of 1-[4-amino-2-(4-methyl-thiazol-2-yl)-thieno[2,3-d]pyrimidin-6-yl]-ethane-1,2-diol as described in example 9.

### Example 34

### Step b

### 2-(5-Methyl-furan-2-yl)-6-piperidin-1-ylmethyl-thieno[2,3-d]pyrimidin-4-ylamine

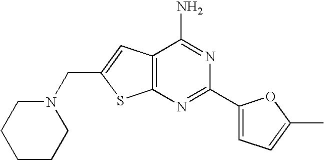

The title compound was prepared using piperidine and 4-amino-2-(5-methyl-furan-2-yl)-thieno[2,3-d]pyrimidine-6-carbaldehyde in place of cis-2,6-dimethyl-piperidine and 4-amino-2-(5-tert-butyl-thiophen-2-yl)-thieno[2,3-d]pyrimidine-6-carbaldehyde, respectively, as described in Example 13. ¹H NMR (CHLOROFORM-d, 400 MHz): δ=7.09 (d, J=3.0 Hz, 1H), 6.90 (s, 1H), 6.08 (d, J=2.3 Hz, 1H), 5.21 (br. s., 2H), 3.65 (s, 2H), 2.42 (br. s., 4H), 2.38 (s, 3H), 1.55 (quin, J=5.6 Hz, 4H), 1.39 ppm (d, J=5.1 Hz, 2H); MS m/e 329 (M+H).

### Example 35

### 2-(5-Bromo-furan-2-yl)-6-pyrrolidin-1-ylmethyl-thieno[2,3-d]pyrimidin-4-ylamine

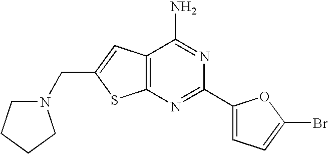

The title compound was prepared using pyrrolidine and 5-bromo-furan-2-carbonitrile in place of cis-2,6-dimethyl-piperidine and 5-tert-butyl-thiophene-2-carbonitrile, respectively, as described in Example 13. ¹H NMR (CHLOROFORM-d, 300 MHz): δ=7.20 (d, J=3.4 Hz, 1H), 7.07 (s, 1H), 6.47 (d, J=3.6 Hz, 1H), 5.47 (br. s., 2H), 3.91 (s, 2H), 2.61-2.74 (m, 4H), 1.85 ppm (dt, J=6.7, 3.2 Hz, 4H); MS m/e 380 (M+H).

### Example 36

### 2-(5-Methyl-furan-2-yl)-6-(4-methyl-piperazin-1-ylmethyl)-thieno[2,3-d]pyrimidin-4-ylamine

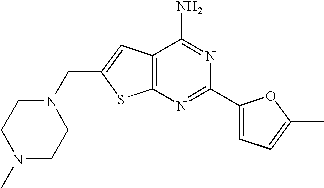

The title compound was prepared using 1-methyl-piperazine in place of piperidine as described in Example 34. ¹H NMR (CHLOROFORM-d, 300 MHz): δ=7.16 (d, J=3.2 Hz, 1H), 6.94 (s, 1H), 6.15 (dd, J=3.2, 0.9 Hz, 1H), 5.33 (br. s., 2H), 3.74 (s, 2H), 2.47-2.69 (m, 8H), 2.45 (s, 3H), 2.33 ppm (s, 3H); MS m/e 344 (M+H).

### Example 37

### 6-(1,3-Dihydro-isoindol-2-ylmethyl)-2-(5-methyl-furan-2-yl)-thieno[2,3-d]pyrimidin-4-ylamine

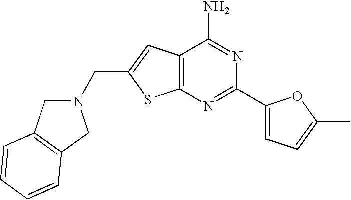

The title compound was prepared using 2,3-dihydro-1H-isoindole in place of piperidine as described in Example 34. ¹H NMR (CHLOROFORM-d, 300 MHz): δ=7.20 (s, 4H), 7.17 (d, J=3.2 Hz, 1H), 7.03 (s, 1H), 6.11-6.18 (m, 1H), 5.30 (br. s., 2H), 4.13 (s, 2H), 4.04 (s, 4H), 2.45 ppm (s, 3H); MS m/e 363 (M+H).

### Example 38

### 2-(5-Methyl-furan-2-yl)-6-morpholin-4-ylmethyl-thieno[2,3-d]pyrimidin-4-ylamine

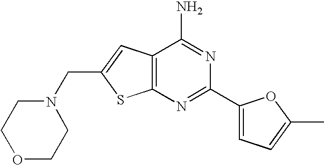

The title compound was prepared using morpholine in place of piperidine as described in Example 34. ¹H NMR (CHLOROFORM-d, 400 MHz): δ=7.17 (d, J=3.3 Hz, 1H), 6.95 (s, 1H), 6.15 (d, J=2.5 Hz, 1H), 5.27 (br. s., 2H), 3.61-3.80 (m, 6H), 2.49-2.58 (m, 4H), 2.45 ppm (s, 3H); MS m/e 331 (M+H).

### Example 39

### 2-(5-Methyl-furan-2-yl)-6-pyrrolidin-1-ylmethyl-thieno[2,3-d]pyrimidin-4-ylamine

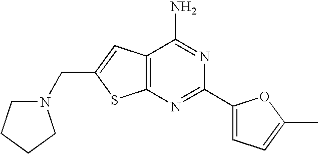

The title compound was prepared using pyrrolidine in place of piperidine as described in Example 34. ¹H NMR (CHLOROFORM-d, 300 MHz): δ=7.17 (d, J=3.4 Hz, 1H), 7.11 (s, 1H), 6.15 (d, J=3.4 Hz, 1H), 5.50 (br. s., 2H), 3.94 (s, 2H), 2.66-2.80 (m, 4H), 2.44 (s, 3H), 1.86 ppm (dt, J=6.4, 3.3 Hz, 4H); MS m/e 315 (M+H).

### Example 40

### 1-[4-Amino-2-(5-methyl-furan-2-yl)-thieno[2,3-d]pyrimidin-6-ylmethyl]-pyrrolidin-3-ol

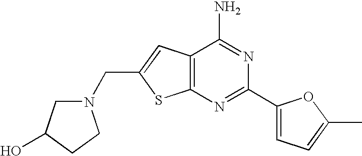

The title compound was prepared using pyrrolidin-3-ol in place of piperidine as described in Example 34. ¹H NMR (CHLOROFORM-d, 300 MHz): δ=7.17 (d, J=3.2 Hz, 1H), 6.85-7.02 (m, 1H), 6.06-6.25 (m, 1H), 5.25 (br. s., 2H), 4.26-4.45 (dddd, J=7.0, 4.8, 2.3, 2.3 Hz, 1H), 3.88 (s, 2H), 2.96 (td, J=8.6, 5.7 Hz, 1H), 2.72-2.79 (m, 1H), 2.59-2.69 (m, 1H), 2.38-2.50 (m, 1H), 2.45 (s, 3H), 2.12-2.30 ppm (m, 2H); MS m/e 331 (M+H).

### Example 41

### 6-(3,4-Dihydro-1H-isoquinolin-2-ylmethyl)-2-(5-methyl-furan-2-yl)-thieno[2,3-d]pyrimidin-4-ylamine

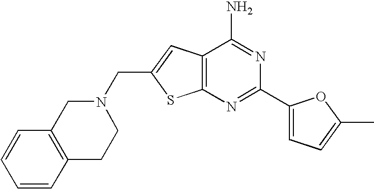

The title compound was prepared using 1,2,3,4-tetrahydro-isoquinoline in place of piperidine as described in Example 34. ¹H NMR (CHLOROFORM-d, 300 MHz): δ=7.06-7.23 (m, 4 H), 6.95-7.04 (m, 2H), 6.14 (dd, J=3.3, 0.8 Hz, 1H), 5.35 (br. s., 2H), 3.91 (s, 2H), 3.74 (s, 2H), 2.91 (t, J=5.3 Hz, 3H), 2.82 ppm (t, J=5.5 Hz, 2H); MS m/e 377 (M+H).

### Example 42

### 2-Furan-2-yl-6-morpholin-4-ylmethyl-thieno[2,3-d]pyrimidin-4-ylamine

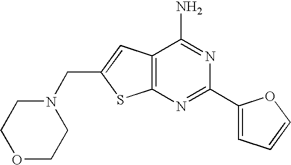

The title compound was prepared using morpholine and 2-furonitrile in place of cis-2,6-dimethyl-piperidine and 5-tert-butyl-thiophene-2-carbonitrile, respectively, as described in Example 13. ¹H NMR (DMSO-d₆, 300 MHz): δ=7.81 (s, 1H), 7.50 (s, 2H), 7.41 (s, 1H), 7.11 (d, J=3.4 Hz, 1H), 6.51-6.72 (m, 1H), 3.71 (s, 2H), 3.60 (t, J=4.3 Hz, 4H), 2.44 ppm (br. s., 4H); MS m/e 317 (M+H).

### Example 43

### 2-Cyclopropyl-6-pyrrolidin-1-ylmethyl-thieno[2,3-d]pyrimidin-4-ylamine

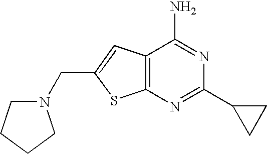

The title compound was prepared using pyrrolidine and cyclopropylnitrile in place of cis-2,6-dimethyl-piperidine and 5-tert-butyl-thiophene-2-carbonitrile, respectively, as described in Example 13. ¹H NMR (CHLOROFORM-d, 300 MHz): δ=7.07 (s, 1H), 5.59 (br. s., 2H), 3.92 (s, 2H), 2.59-2.86 (m, 4H), 2.00-2.16 (m, 1H), 1.86 (dt, J=6.7, 3.3 Hz, 4H), 1.07-1.18 (m, 2H), 0.90-1.02 ppm (m, 2H); MS m/e 275 (M+H).

### Example 44

### 6-(2,6-Dimethyl-piperidin-1-ylmethyl)-2-furan-2-yl-thieno[2,3-d]pyrimidin-4-ylamine

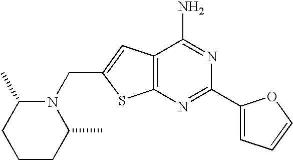

The title compound was prepared using 2-furonitrile in place of 5-tert-butyl-thiophene-2-carbonitrile as described in Example 13. ¹H NMR (CHLOROFORM-d, 300 MHz): δ=7.59 (s, 1H), 7.25 (d, J=3.4 Hz, 1H), 6.95 (s, 1H), 6.55 (dd, J=3.4, 1.5 Hz, 1H), 5.32 (br. s., 2H), 4.11 (s, 2H), 2.56 (br. s., 2H), 1.76 (br. s., 2H), 1.52-1.70 (m, 4H), 1.21 (s, 3H), 1.19 ppm (s, 3H); MS m/e 343 (M+H).

### Example 45

### 1-(4-Amino-2-furan-2-yl-thieno[2,3-d]pyrimidin-6-ylmethyl)-piperidin-4-one

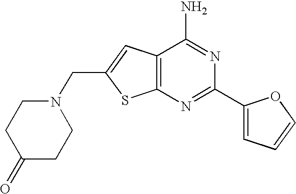

The title compound was prepared using 4-piperidone monohydrate hydrochloride and 2-furonitrile in place of cis-2,6-dimethyl-piperidine and 5-tert-butyl-thiophene-2-carbonitrile, respectively, as described in Example 13. ¹H NMR (CHLOROFORM-d, 300 MHz): δ=7.60 (s, 1H), 7.26 (s, 1H), 7.00 (s, 1H), 6.55 (dd, J=3.4, 1.5 Hz, 1H), 5.47 (s, 2H), 3.86 (s, 2H), 2.84 (t, J=6.0 Hz, 4H), 2.49 ppm (t, J=6.0 Hz, 4H); MS m/e 329 (M+H).

### Example 46

### 6-Dimethylaminomethyl-2-furan-2-yl-thieno[2,3-d]pyrimidin-4-ylamine

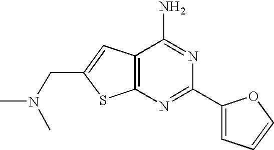

The title compound was prepared using a 2.0 M THF solution of dimethylamine and 2-furonitrile in place of cis-2,6-dimethyl-piperidine and 5-tert-butyl-thiophene-2-carbonitrile, respectively, as described in Example 13. ¹H NMR (CHLOROFORM-d, 300 MHz): δ=7.59 (s, 1H), 7.20-7.33 (m, 1H), 7.12 (s, 1H), 6.55 (dd, J=3.4, 1.9 Hz, 1H), 5.82 (br. s., 2H), 3.78 (s, 2H), 2.38 ppm (s, 6H); MS m/e 275 (M+H).

### Example 47

### 2-(3,5-Difluoro-phenyl)-6-morpholin-4-ylmethyl-thieno[2,3-d]pyrimidin-4-ylamine

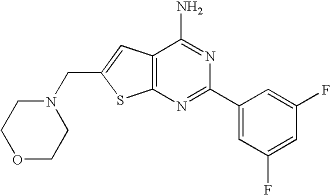

The title compound was prepared using morpholine and 3,5-difluoro-benzonitrile in place of cis-2,6-dimethyl-piperidine and 5-tert-butyl-thiophene-2-carbonitrile, respectively, as described in Example 13. ¹H NMR (CHLOROFORM-d, 300 MHz): δ=7.87-8.05 (m, 2H), 7.00 (s, 1H), 6.87 (tt, J=8.7, 2.4 Hz, 1H), 5.23 (br. s., 2H), 3.59-3.83 (m, 6H), 2.41-2.67 ppm (m, 4H); MS m/e 363 (M+H).

### Example 48

### 2-(3-Chloro-phenyl)-6-(4-fluoro-piperidin-1-ylmethyl)-thieno[2,3-d]pyrimidin-4-ylamine

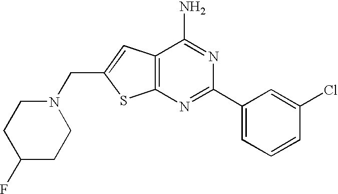

The title compound was prepared using 4-fluoro-piperidine hydrochloride and 3-chloro-benzonitrile in place of cis-2,6-dimethyl-piperidine and 5-tert-butyl-thiophene-2-carbonitrile, respectively, as described in Example 13. ¹H NMR (CHLOROFORM-d, 300 MHz): δ=8.44 (s, 1H), 8.31 (td, J=4.2, 2.1 Hz, 1H), 7.32-7.45 (m, 2H), 6.98 (s, 1H), 5.20 (br. s., 2H), 4.57-4.90 (m, 1H), 3.76 (s, 2H), 2.57-2.73 (m, 2H), 2.40-2.57 (m, 2H), 1.78-2.05 ppm (m, 4H); MS m/e 377 (M+H).

### Example 49

### 2-(3-Chloro-phenyl)-6-morpholin-4-ylmethyl-thieno[2,3-d]pyrimidin-4-ylamine

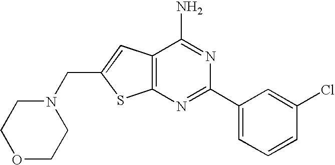

The title compound was prepared using morpholine and 3-chloro-benzonitrile in place of cis-2,6-dimethyl-piperidine and 5-tert-butyl-thiophene-2-carbonitrile, respectively, as described in Example 13. ¹H NMR (CHLOROFORM-d, 300 MHz): δ=8.44 (s, 1H), 8.31 (dt, J=6.2, 2.2 Hz, 1H), 7.33-7.47 (m, 2H), 6.99 (s, 1H), 5.25 (br. s., 2H), 3.67-3.85 (m, 6H), 2.44-2.63 ppm (m, 4H); MS m/e 361 (M+H).

### Example 50

### 2-(3,4-Difluoro-phenyl)-6-morpholin-4-ylmethyl-thieno[2,3-d]pyrimidin-4-ylamine

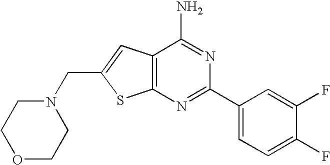

The title compound was prepared using morpholine and 3,4-difluoro-benzonitrile in place of cis-2,6-dimethyl-piperidine and 5-tert-butyl-thiophene-2-carbonitrile, respectively, as described in Example 13. ¹H NMR (CHLOROFORM-d, 300 MHz): δ=8.28 (ddd, J=11.9, 7.9, 2.1 Hz, 1H), 8.20 (ddd, J=8.7, 4.5, 1.5 Hz, 1H), 7.15-7.25 (m, 1H), 6.99 (s, 1H), 5.19 (br. s., 2H), 3.52-3.88 (m, 6H), 2.46-2.62 ppm (m, 4H); MS m/e 363 (M+H).

### Example 51

### 6-(3-Fluoro-pyrrolidin-1-ylmethyl)-2-furan-2-yl-thieno[2,3-d]pyrimidin-4-ylamine

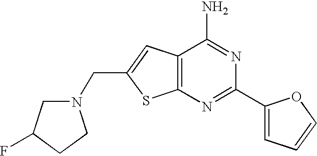

The title compound was prepared using 3-fluoro-pyrrolidine and 2-furonitrile in place of cis-2,6-dimethyl-piperidine and 5-tert-butyl-thiophene-2-carbonitrile, respectively, as described in Example 13. ¹H NMR (CHLOROFORM-d, 300 MHz): δ=7.60 (s, 1H), 7.25 (d, J=3.4 Hz, 1H), 6.99 (s, 1H), 6.55 (dd, J=3.4, 1.9 Hz, 1H), 5.34 (br. s., 2H), 5.20-5.32 (m, 1H), 3.92 (s, 2H), 2.93-2.98 (m, 1H), 2.82-2.94 (m, 2H), 2.57-2.68 (m, 1H), 2.06-2.27 ppm (m, 2H); MS m/e 319 (M+H).

### Example 52

### 1-(4-Amino-2-furan-2-yl-thieno[2,3-d]pyrimidin-6-ylmethyl)-piperidin-4-ol

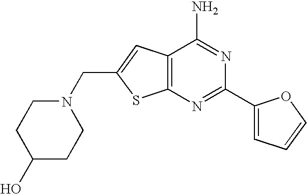

The title compound was prepared using piperidin-4-ol and 2-furonitrile in place of cis-2,6-dimethyl-piperidine and 5-tert-butyl-thiophene-2-carbonitrile, respectively, as described in Example 13. ¹H NMR (CHLOROFORM-d, 300 MHz): δ=7.60 (s, 1H), 7.25 (s, 1H), 6.99 (s, 1H), 6.55 (dd, J=3.4, 1.9 Hz, 1H), 5.33 (br. s., 2H), 3.85 (s, 2H), 2.75 (t, J=11.1 Hz, 2H), 2.56 (t, J=5.3 Hz, 2H), 1.70-1.99 ppm (m, 5H); MS m/e 331 (M+H).

### Example 53

### 6-(3-Fluoro-pyrrolidin-1-ylmethyl)-2-furan-2-yl-thieno[2,3-d]pyrimidin-4-ylamine

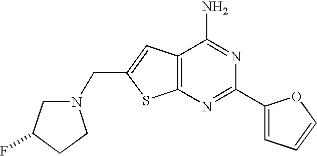

The title compound was prepared using (S)-3-fluoro-pyrrolidine and 2-furonitrile in place of cis-2,6-dimethyl-piperidine and 5-tert-butyl-thiophene-2-carbonitrile, respectively, as described in Example 13. ¹H NMR (CHLOROFORM-d, 300 MHz): δ=7.60 (s, 1H), 7.25 (d, J=3.4 Hz, 1H), 6.99 (s, 1H), 6.55 (dd, J=3.4, 1.9 Hz, 1H), 5.34 (br. s., 2H), 5.20-5.32 (m, 1H), 3.92 (s, 2H), 2.93-2.98 (m, 1H), 2.82-2.94 (m, 2H), 2.57-2.68 (m, 1H), 2.06-2.27 ppm (m, 2H); MS m/e 319 (M+H).

### Example 54

### 6-(3-Fluoro-pyrrolidin-1-ylmethyl)-2-furan-2-yl-thieno[2,3-d]pyrimidin-4-ylamine

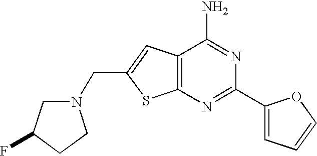

The title compound was prepared using (R)-3-fluoro-pyrrolidine and 2-furonitrile in place of cis-2,6-dimethyl-piperidine and 5-tert-butyl-thiophene-2-carbonitrile, respectively, as described in Example 13. ¹H NMR (CHLOROFORM-d, 300 MHz): δ=7.60 (s, 1H), 7.25 (d, J=3.4 Hz, 1H), 6.99 (s, 1H), 6.55 (dd, J=3.4, 1.9 Hz, 1H), 5.34 (br. s., 2H), 5.20-5.32 (m, 1H), 3.92 (s, 2H), 2.93-2.98 (m, 1H), 2.82-2.94 (m, 2H), 2.57-2.68 (m, 1H), 2.06-2.27 ppm (m, 2H); MS m/e 319 (M+H).

### Example 55

### 2-(3,5-Difluoro-phenyl)-6-(4-fluoro-piperidin-1-ylmethyl)-thieno[2,3-d]pyrimidin-4-ylamine

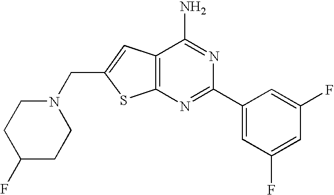

The title compound was prepared using 4-fluoro-piperidine hydrochloride and 3,5-difluoro-benzonitrile in place of cis-2,6-dimethyl-piperidine and 5-tert-butyl-thiophene-2-carbonitrile, respectively, as described in Example 13. ¹H NMR (CHLOROFORM-d, 300 MHz): δ=7.98 (d, J=9.0 Hz, 2H), 6.98 (s, 1H), 6.68-6.93 (m, 1H), 5.20 (br. s., 2H), 4.60-4.87 (m, 1H), 3.77 (s, 2H), 2.46-2.72 (m, 4H), 1.79-2.10 ppm (m, 4H); MS m/e 379 (M+H).

### Example 56

### 6-(4,4-Difluoro-piperidin-1-ylmethyl)-2-furan-2-yl-thieno[2,3-d]pyrimidin-4-ylamine

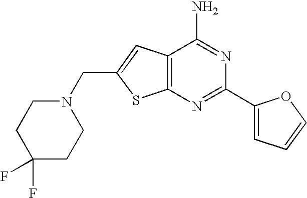

The title compound was prepared using 4,4-difluoro-piperidine hydrochloride and 2-furonitrile in place of cis-2,6-dimethyl-piperidine and 5-tert-butyl-thiophene-2-carbonitrile, respectively, as described in Example 13. ¹H NMR (CHLOROFORM-d, 300 MHz): δ=7.59 (s, 1H), 7.26 (d, J=3.4 Hz, 1H), 6.96 (s, 1H), 6.55 (dd, J=3.4, 1.5 Hz, 1H), 5.46 (s, 2H), 3.78 (s, 2H), 2.63 (t, J=5.5 Hz, 4H), 1.91-2.11 (m, 4H); MS m/e 351 (M+H).

### Example 57

### 6-(3,3-Difluoro-piperidin-1-ylmethyl)-2-furan-2-yl-thieno[2,3-d]pyrimidin-4-ylamine

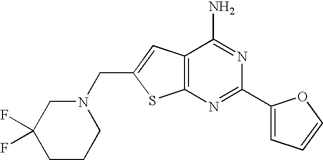

The title compound was prepared using 3,3-difluoro-piperidine hydrochloride and 2-furonitrile in place of cis-2,6-dimethyl-piperidine and 5-tert-butyl-thiophene-2-carbonitrile, respectively, as described in Example 13. ¹H NMR (Acetone, 300 MHz): δ=7.70 (s, 1H), 7.36 (s, 1H), 7.16 (d, J=3.4 Hz, 1H), 6.79 (br. s., 2H), 6.59 (dd, J=3.4, 1.9 Hz, 1H), 3.74 (s, 2H), 3.63 (br. s., 2H), 2.13-2.31 (m, 2H), 1.83 (dd, J=12.6, 3.6 Hz, 2H), 1.46-1.65 ppm (m, 2H); MS m/e 351 (M+H).

### Example 58

### 2-(3-Fluoro-phenyl)-6-thiomorpholin-4-ylmethyl-thieno[2,3-d]pyrimidin-4-ylamine

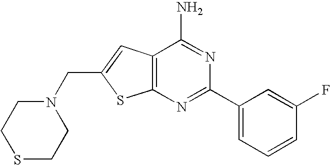

The title compound was prepared using thiomorpholine and 3-fluoro-benzonitrile in place of cis-2,6-dimethyl-piperidine and 5-tert-butyl-thiophene-2-carbonitrile, respectively, as described in Example 13. ¹H NMR (CHLOROFORM-d, 300 MHz): δ=8.24 (d, J=7.9 Hz, 1H), 8.10-8.20 (m, 1H), 7.44 (td, J=7.9, 6.0 Hz, 1H), 7.09-7.20 (m, 1H), 7.01 (s, 1H), 5.22 (br. s., 2H), 3.80 (s, 2H), 2.79-2.91 (m, 4H), 2.67-2.79 ppm (m, 4H); MS m/e 362 (M+H).

### Example 59

### 2-Benzofuran-2-yl-6-(4-fluoropiperidin-1-ylmethyl)thieno[2,3-d]pyrimidin-4-ylamine

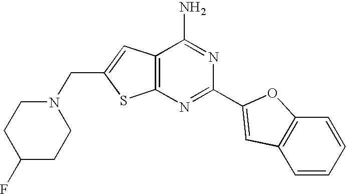

The title compound was prepared using 4-fluoropiperidine hydrochloride and benzofuran-2-carbonitrile in place of cis-2,6-dimethyl-piperidine and 5-tert-butyl-thiophene-2-carbonitrile, respectively, as described in Example 13. ¹H NMR (CHLOROFORM-d, 300 MHz): δ=7.45-7.74 (m, 3H), 7.30 (m, 1H), 7.20 (m, 1H), 6.94 (br. s., 1H), 5.28 (br. s., 2H), 4.66 (d, J=48.6 Hz, 1H), 4.46-4.67 (m, 1H), 3.71 (s, 2H), 2.38-2.66 (m, 4H), 1.72-1.94 ppm (m, 4H); MS m/e 383 (M+H).

### Example 60

### 2-(3-Fluoro-phenyl)-6-(4-fluoro-piperidin-1-ylmethyl)-thieno[2,3-d]pyrimidin-4-ylamine

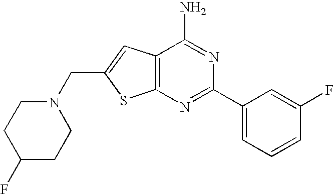

The title compound was prepared using 4-fluoropiperidine hydrochloride and 3-fluoro-benzonitrile in place of cis-2,6-dimethyl-piperidine and 5-tert-butyl-thiophene-2-carbonitrile, respectively, as described in Example 13. ¹H NMR (CHLOROFORM-d, 300 MHz): δ=8.22 (d, J=7.9 Hz, 1H), 8.13 (m, 1H), 7.42 (td, J=8.0, 5.8 Hz, 1H), 7.08-7.19 (m, 1H), 7.02 (br. s., 1H), 5.22 (br. s., 2H), 4.74 (d, J=48.6 Hz, 1H), 3.79 (s, 2H), 2.65 (m, 4H), 1.80-2.03 ppm (m, 4H); MS m/e 361 (M+H).

### Example 61

### 2-(3-Fluoro-phenyl)-6-morpholin-4-ylmethyl-thieno[2,3-d]pyrimidin-4-ylamine

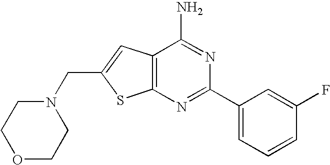

The title compound was prepared using morpholine and 3-fluoro-benzonitrile in place of cis-2,6-dimethyl-piperidine and 5-tert-butyl-thiophene-2-carbonitrile, respectively, as described in Example 13. ¹H NMR (CHLOROFORM-d, 300 MHz): δ=8.24 (d, J=7.9 Hz, 1H), 8.15 (dt, J=10.5, 2.1 Hz, 1H), 7.44 (td, J=8.0, 5.8 Hz, 1H), 7.07-7.22 (m, 1H), 7.03 (s, 1H), 5.23 (br. s., 2H), 3.69-3.84 (m, 6H), 2.51-2.63 ppm (m, 4H); MS m/e 345 (M+H).

### Example 62

### 3-[4-Amino-6-(3,3-difluoro-piperidin-1-ylmethyl)-thieno[2,3-d]pyrimidin-2-yl]-benzonitrile

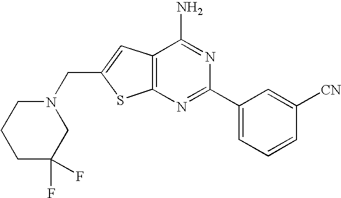

The title compound was prepared using 3,3-difluoropiperidine hydrochloride and 1,3-dicyanobenzene in place of cis-2,6-dimethyl-piperidine and 5-tert-butyl-thiophene-2-carbonitrile, respectively, as described in Example 13. 1H NMR (300 MHz, CHLOROFORM-D) δ ppm 8.77 (t, J=1.5 Hz, 1H), 8.68 (dt, J=7.9, 1.5 Hz, 1H), 7.70 (dt, J=7.6, 1.5 Hz, 1H), 7.56 (t, J=7.9 Hz, 1H), 7.04 (s, 1H), 5.27 (br s, 2H), 3.89 (s, 2H), 2.76 (t, J_{HF}=11.1 Hz, 2H), 2.55-2.63 (m, 2H), 1.77-1.99 (m, 4H); MS m/e 386 (M+H).

### Example 63

### 4-[4-Amino-6-(3,6-dihydro-2H-pyridin-1-ylmethyl)-thieno[2,3-d]pyrimidin-2-yl]-benzonitrile

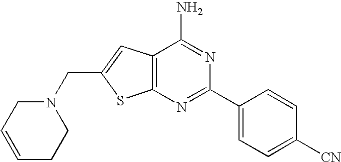

The title compound was prepared using 1,2,3,6-tetrahydropyridine and 1,4-dicyanobenzene in place of cis-2,6-dimethyl-piperidine and 5-tert-butyl-thiophene-2-carbonitrile, respectively, as described in Example 13. 1H NMR (300 MHz, CHLOROFORM-D) δ ppm 8.56 (d, J=8.3 Hz, 2H), 7.74 (d, J=8.3 Hz, 2H), 7.05 (s, 1H), 5.76-5.84 (m, 1H), 5.62-5.73 (m, 1H), 5.22 (br s, 2H), 3.86 (s, 2H), 3.08-3.13 (m, 2H), 2.66 (t, J=5.7 Hz, 2H), 2.17-2.24 (m, 2H), MS m/e 348 (M+H).

### Example 64

### 3-[4-Amino-6-(2,5-dihydro-pyrrol-1-ylmethyl)-thieno[2,3-d]pyrimidin-2-yl]-benzonitrile

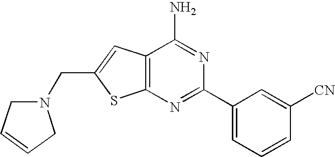

The title compound was prepared using 3-pyrroline and 1,3-dicyanobenzene in place of cis-2,6-dimethyl-piperidine and 5-tert-butyl-thiophene-2-carbonitrile, respectively, as described in Example 13. 1H NMR (300 MHz, DMSO-D6) δ ppm 8.60-8.69 (m, 2H), 7.91-7.99 (m, 1H), 7.70 (t, J=7.7 Hz, 1H), 7.60 (br s, 2H), 7.47 (s, 1H), 5.83 (s, 2H), 4.03 (s, 2H), 3.52 (s, 4H); MS m/e 334 (M+H).

### Example 65

### 3-(4-Amino-6-pyrrolidin-1-ylmethyl-thieno[2,3-d]pyrimidin-2-yl)-benzonitrile

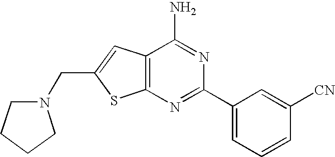

The title compound was prepared using pyrrolidine and 1,3-dicyanobenzene in place of cis-2,6-dimethyl-piperidine and 5-tert-butyl-thiophene-2-carbonitrile, respectively, as described in Example 13. 1H NMR (300 MHz, CHLOROFORM-D) δ ppm 8.76 (s, 1H), 8.67 (dt, J=7.9, 1.3 Hz, 1H), 7.69 (dt, J=7.6, 1.5 Hz, 1H), 7.55 (t, J=7.7 Hz, 1H), 7.03 (s, 1H), 5.32 (br s, 2H), 3.90 (s, 2H), 2.58-2.68 (m, 4H), 1.77-1.89 (m, 4H); MS m/e 336 (M+H).

### Example 66

### 4-[4-Amino-6-(4-fluoro-piperidin-1-ylmethyl)-thieno[2,3-d]pyrimidin-2-yl]-benzonitrile

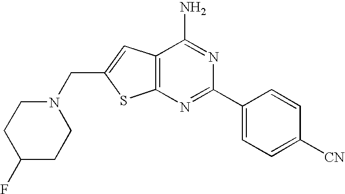

The title compound was prepared using 4-fluoropiperidine hydrochloride and 1,4-dicyanobenzene in place of cis-2,6-dimethyl-piperidine and 5-tert-butyl-thiophene-2-carbonitrile, respectively, as described in Example 13. 1H NMR (300 MHz, CHLOROFORM-D) δ ppm 8.55 (d, J=8.7 Hz, 2H), 7.74 (d, J=8.7 Hz, 2H), 7.01 (s, 1H), 5.25 (s, 2H), 4.74 (d, J_{HF}=48.6 Hz, 1H), 3.78 (s, 2H), 2.48-2.69 (m, 4H), 1.85-1.99 (m, 4H); MS m/e 368 (M+H).

### Example 67

### 4-(4-Amino-6-azepan-1-ylmethyl-thieno[2,3-d]pyrimidin-2-yl)-benzonitrile

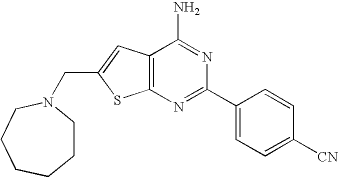

The title compound was prepared using hexamethyleneimine and 1,4-dicyanobenzene in place of cis-2,6-dimethyl-piperidine and 5-tert-butyl-thiophene-2-carbonitrile, respectively, as described in Example 13. 1H NMR (300 MHz, CHLOROFORM-D) δ ppm 8.55 (d, J=8.3 Hz, 2H), 7.74 (d, J=8.3 Hz, 2H), 6.98 (s, 1H), 5.24 (br s, 2H), 3.90 (s, 2H), 2.65-2.75 (m, 4H), 1.64 (m, 8H); MS m/e 364 (M+H).

### Example 68

### 3-[4-Amino-6-(3,6-dihydro-2H-pyridin-1-ylmethyl)-thieno[2,3-d]pyrimidin-2-yl]-benzonitrile

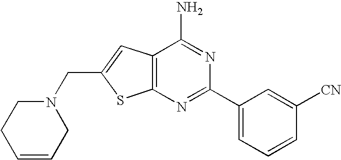

The title compound was prepared using 1,2,3,6-tetrahydropyridine and 1,3-dicyanobenzene in place of cis-2,6-dimethyl-piperidine and 5-tert-butyl-thiophene-2-carbonitrile, respectively, as described in Example 13. 1H NMR (300 MHz, CHLOROFORM-D) δ ppm 8.75 (t, J=1.5 Hz, 1H), 8.66 (ddd, J=8.1, 1.3, 1.1 Hz, 1H), 7.69 (ddd, J=7.7, 1.3, 1.1 Hz, 1H), 7.55 (t, J=7.7 Hz, 1H), 7.06 (s, 1H), 5.75-5.82 (m, 1H), 5.64-5.71 (m, 1H), 5.39 (br s, 2H), 3.86 (s, 2H), 3.06-3.15 (m, 2H), 2.67 (t, J=5.7 Hz, 2H), 2.17-2.24 (m, 2H); MS m/e 348 (M+H).

### Example 69

### 6-(2,5-Dihydro-pyrrol-1-ylmethyl)-2-oxazol-4-yl-thieno[2,3-d]pyrimidin-4-ylamine

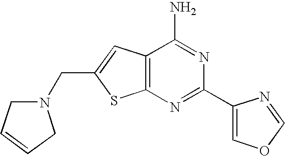

The title compound was prepared using 3-pyrroline and 4-oxazolecarbonitrile in place of cis-2,6-dimethyl-piperidine and 5-tert-butyl-thiophene-2-carbonitrile, respectively, as described in Example 13. 1H NMR (300 MHz, MeOD) δ ppm 8.51 (s, 1H), 8.28 (s, 1H), 7.34 (s, 1H), 5.83 (s, 2H), 4.13 (s, 2H), 3.63 (s, 4H); MS m/e 300 (M+H).

### Example 70

### 6-(3,6-Dihydro-2H-pyridin-1-ylmethyl)-2-oxazol-4-yl-thieno[2,3-d]pyrimidin-4-ylamine

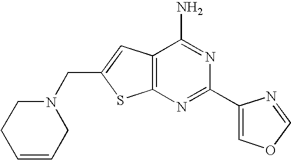

The title compound was prepared using 1,2,3,6-tetrahydropyridine and 4-oxazolecarbonitrile in place of cis-2,6-dimethyl-piperidine and 5-tert-butyl-thiophene-2-carbonitrile, respectively, as described in Example 13. 1H NMR (300 MHz, CHLOROFORM-D) δ ppm 8.41 (s, 1H), 7.98 (s, 1H), 7.04 (s, 1H), 5.74-5.83 (m, 1H), 5.64-5.71 (m, 1H), 5.44 (br s, 2H), 3.85 (s, 2H), 3.06-3.12 (m, 2H), 2.65 (t, J=5.7 Hz, 2H), 2.15-2.24 (m, 2H); MS m/e 314 (M+H).

### Example 71

### 6-(4-Fluoro-piperidin-1-ylmethyl)-2-(3-methoxy-phenyl)-thieno[2,3-d]pyrimidin-4-ylamine

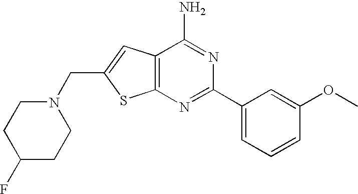

The title compound was prepared using 4-fluoropiperidine hydrochloride and 3-methoxybenzonitrile in place of cis-2,6-dimethyl-piperidine and 5-tert-butyl-thiophene-2-carbonitrile, respectively, as described in Example 13. 1H NMR (300 MHz, CHLOROFORM-D) δ ppm 8.02 (dt, J=7.6, 1.3 Hz, 1H), 7.99 (dd, J=2.6, 1.5 Hz, 1H), 7.37 (t, J=7.9 Hz, 1H), 6.96-7.02 (m, 2H), 5.18 (br s, 2H), 4.72 (d, J_{HF}=49.0 Hz, 1H), 3.92 (s, 3H), 3.76 (s, 2H), 2.59-2.69 (m, 2H), 2.47-2.57 (m, 2H), 1.85-1.99 (m, 4H); MS m/e 373 (M+H).

### Example 72

### 5-[4-Amino-6-(4-fluoro-piperidin-1-ylmethyl)-thieno[2,3-d]pyrimidin-2-yl]-thiophene-2-carbonitrile

### Example 72

### Step a

### 6-Methyl-2-methylsulfanyl-thieno[2,3-d]pyrimidin-4-ylamine

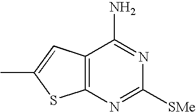

Solid 2-amino-5-methylthiophene-3-carbonitrile (6.0 g, 43.5 mmol, 1 equiv) was added a 4 M solution of hydrogen chloride in 1,4-dioxane (60 mL) followed by methyl thiocyanate (2.98 mL, 43.5 mmol, 1 equiv). The resulting suspension was heated to 70° C. in a sealed pressure tube for 24 h. The mixture was allowed to cool to 23° C. and the brown solid precipitate was collected by vacuum filtration. The solid was partitioned between EtOAc and a saturated aqueous NaHCO₃. The aqueous phase was extracted with EtOAc. The organic extracts were dried (Na₂SO₄), filtered, and concentrated, yielding a brown solid (5.4 g). An additional 2.5 g of crude product was collected by filtration of the aqueous phase. The two batches of 6-methyl-2-(methylthio)thieno[2,3-d]pyrimidin-4-amine were combined and used without further purification.

### Example 72

### Step b

### 4-Amino-2-methylsulfanyl-thieno[2,3-d]pyrimidine-6-carbaldehyde

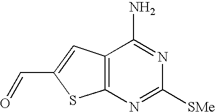

Solid SeO₂ (12.2 g, 109.7 mmol, nominally 3 equiv) was added to a dioxane (250 mL)/water (2 mL) suspension of the crude 6-methyl-2-methylsulfanyl-thieno[2,3-d]pyrimidin-4-ylamine (7.7 g) and was heated to reflux. After 23 h, and an additional portion of selenium dioxide (4.1 g) was added and the mixture continued to reflux. After 24 h the precipitated solids were removed by filtration and the filtrate was concentrated. The residual solid (17.5 g), consisting of crude 4-amino-2-methylsulfanyl-thieno[2,3-d]pyrimidine-6-carbaldehyde, was used without further purification.

### Example 72

### Step c

### 6-(4-Fluoro-piperidin-1-ylmethyl)-2-methylsulfanyl-thieno[2,3-d]pyrimidin-4-ylamine

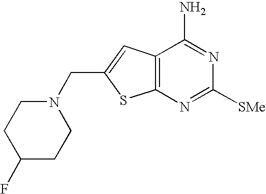

Solid NaBH(OAc)₃ (3.1 g, 14.4 mmol) was added to a THF solution (80 mL) of crude 4-amino-2-methylsulfanyl-thieno[2,3-d]pyrimidine-6-carbaldehyde (4.3 g) and 4-fluoropiperidine hydrochloride (2.7 g, 19.3 mmol) and the resulting mixture was heated to 40° C. After 3 days, TLC analysis indicated remaining starting aldehyde; additional portions of the amine hydrochloride and sodium triacetoxyborohydride (one-half of amounts above) were added. After stirring for 3 h, an additional 1.5 g sodium triacetoxyborohydride was added, resulting in consumption of the aldehyde after 1 h at 40° C. Excess hydride reagent was quenched by addition of water (3 mL). The mixture was concentrated and the residue was partitioned between EtOAc and saturated aqueous NaHCO₃. The aqueous phase was extracted with EtOAc and the combined organic extracts were washed with saturated aqueous NaCl. The organic phase was dried (Na₂SO₄), filtered and concentrated and the residue was purified by flash column chromatography (SiO₂, gradient 60-100% EtOAc-heptane), affording 782 mg of the title compound.

### Example 72

### Step d

### [6-(4-Fluoro-piperidin-1-ylmethyl)-2-methylsulfanyl-thieno[2,3-d]pyrimidin-4-yl]-bis-carbamic acid tert-butyl ester

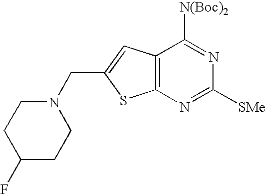

Solid DMAP (37 mg, 0.30 mmol) was added to a THF solution (8 mL) of 6-(4-fluoro-piperidin-1-ylmethyl)-2-methylsulfanyl-thieno[2,3-d]pyrimidin-4-ylamine (951 mg, 3.04 mmol) and (Boc)₂O (1.7 g, 7.61 mmol) and the solution stirred at rt. After 2.5 h the reaction mixture was concentrated and the residue was purified by column chromatography to give 1.24 g of the title compound. 1H NMR (300 MHz, CHLOROFORM-d) δ ppm 6.89 (s, 1H), 4.72 (dm, J_{HF}=48.6 Hz, 1H), 3.74 (s, 2H), 2.58-2.72 (m, 5H), 2.45-2.56 (m, 2H), 1.84-1.99 (m, 4H), 1.43 (s, 18H).

### Example 72

### Step e

### 5-[4-Amino-6-(4-fluoro-piperidin-1-ylmethyl)-thieno[2,3-d]pyrimidin-2-yl]-thiophene-2-carbonitrile

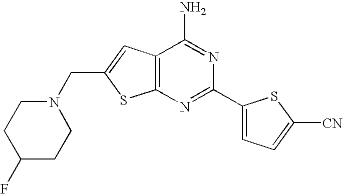

(21): A pressure tube was charged with [6-(4-fluoro-piperidin-1-ylmethyl)-2-methylsulfanyl-thieno[2,3-d]pyrimidin-4-yl]-bis-carbamic acid tert-butyl ester (54 mg, 0.11 mmol), 5-cyanothiophene-2-boronic acid (32 mg, 0.21 mmol), copper(I) thiophene-2-carboxylate (40 mg, 0.212 mmol) and Pd(dppf)Cl₂ (9 mg, 0.01 mmol). The vessel was evacuated and purged with nitrogen (3 cycles), then 1,4-dioxane (0.5 mL) was added. The sealed tube was heated in an 80° C. oil bath. Additional portions of the boronic acid, and copper and palladium catalysts (amounts as above) were added after total reaction times of 16 h and 22 h. After a total reaction time of 2 d, the reaction mixture was diluted with ethyl acetate and was filtered to remove precipitated solids. The filtrate was washed with 10% aqueous ammonium hydroxide (3×50 mL) and the organic phase was dried (Na₂SO₄), filtered, and concentrated. The residue was purified by column chromatography, dissolved in dichloromethane (3 mL) and trifluoroacetic acid (3 mL) and the mixture was stirred at 23° C. for 20 min. The mixture was concentrated and the residue was partitioned between dichloromethane and saturated aqueous NaHCO₃. The aqueous phase was extracted with dichloromethane and the combined organic extracts were dried (Na₂SO₄), filtered, and concentrated. The residue was purified by column chromatography to give 20 mg of the title compound. ¹H NMR (300 MHz, CHLOROFORM-D) δ ppm 7.87 (d, J=3.8 Hz, 1H), 7.60 (t, J=4.5 Hz, 1H), 7.08 (s, 1H), 5.37 (s, 2H), 4.76 (d, J_{HF}=48.6 Hz, 1H), 3.83 (s, 2H), 2.53-2.77 (m, 4H), 1.86-2.08 (m, 4H); MS m/e 374 (M+H).

### Example 73

### 2-[4-Amino-6-(4-fluoro-piperidin-1-ylmethyl)-thieno[2,3-d]pyrimidin-2-yl]-benzonitrile

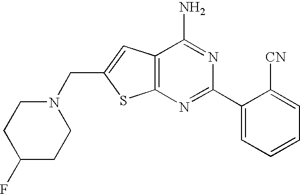

The title compound was prepared using 2-cyanobenzeneboronic acid in place of 5-cyanothiophene-2-boronic acid as described in Example 85. 1H NMR (400 MHz, CHLOROFORM-D) δ ppm 8.36 (d, J=8.1 Hz, 1H), 7.81 (d, J=7.6 Hz, 1H), 7.67 (td, J=7.8, 1.3 Hz, 1H), 7.48-7.53 (m, 1H), 7.05 (s, 1H), 5.36 (s, 2H), 4.74 (d, J_{HF}=48.7 Hz, 1H), 3.79 (s, 2H), 2.47-2.70 (m, 4H), 1.83-2.02 (m, 4H); MS m/e 368 (M+H).

### Example 74

### 3-(4-Amino-6-morpholin-4-ylmethyl-thieno[2,3-d]pyrimidin-2-yl)-benzonitrile

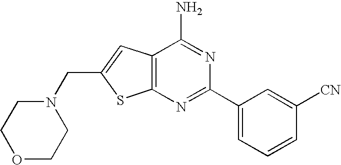

The title compound was prepared using morpholine and 1,3-dicyanobenzene in place of cis-2,6-dimethyl-piperidine and 5-tert-butyl-thiophene-2-carbonitrile, respectively, as described in Example 13. 1H NMR (300 MHz, CHLOROFORM-D) δ ppm 8.76 (s, 1H), 8.67 (dt, J=7.9, 1.5 Hz, 1H), 7.70 (dt, J=7.8, 1.4 Hz, 1H), 7.55 (t, J=7.9 Hz, 1H), 7.03 (s, 1H), 5.34 (br s, 2H), 3.71-3.81 (m, 6H), 2.51-2.63 (m, 4H); MS m/e 352 (M+H).

### Example 75

### 6-Morpholin-4-ylmethyl-2-oxazol-2-yl-thieno[2,3-d]pyrimidin-4-ylamine

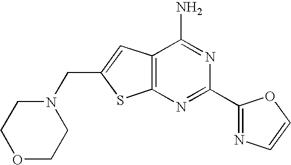

The title compound was prepared using morpholine hydrochloride and 2-oxazolecarbonitrile in place of cis-2,6-dimethyl-piperidine and 5-tert-butyl-thiophene-2-carbonitrile, respectively, as described in Example 13. 1H NMR (300 MHz, METHANOL-D4) δ ppm 8.10 (s, 1H), 7.41 (s, 1H), 7.35 (s, 1H), 3.79 (s, 2H), 3.68-3.73 (m, 4H), 2.49-2.59 (m, 4H); MS m/e 318 (M+H).

### Example 76

### 2-Benzo[1,3]dioxol-5-yl-6-(4-fluoro-piperidin-1-ylmethyl)-thieno[2,3-d]pyrimidin-4-ylamine

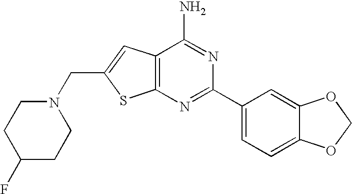

The title compound was prepared using 3,4-methylenedioxybenzeneboronic acid in place of 5-cyanothiophene-2-boronic acid as described in Example 85. 1H NMR (300 MHz, CHLOROFORM-D) δ ppm 8.02 (dd, J=8.3, 1.9 Hz, 1H), 7.92 (d, J=1.5 Hz, 1H), 6.94 (s, 1H), 6.89 (d, J=8.3 Hz, 1H), 6.02 (s, 2H), 5.18 (s, 2H), 4.72 (d, J_{HF}=49.0 Hz, 1H), 3.74 (s, 2H), 2.58-2.68 (m, 2H), 2.46-2.55 (m, 2H), 1.83-2.03 (m, 4H); MS m/e 387 (M+H).

### Example 77

### 3-[4-Amino-6-(7-aza-bicyclo[2.2.1]hept-7-ylmethyl)-thieno[2,3-d]pyrimidin-2-yl]-benzonitrile

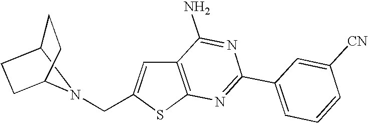

The title compound was prepared using 7-azabicyclo[2.2.1]heptane hydrochloride and 1,3-dicyanobenzene in place of cis-2,6-dimethyl-piperidine and 5-tert-butyl-thiophene-2-carbonitrile, respectively, as described in Example 13. 1H NMR (400 MHz, DMSO-D6) δ ppm 8.60-8.66 (m, 2H), 7.92 (ddd, J=7.7, 1.5, 1.3 Hz, 1H), 7.69 (t, J=7.8 Hz, 1H), 7.55 (br s, 2H), 7.40 (s, 1H), 3.74 (s, 2H), 3.26 (s, 2H), 1.71 (d, J=5.9 Hz, 4H), 1.29 (d, J=6.6 Hz, 4H); MS m/e 362 (M+H).

### Example 78

### 6-(4-Fluoro-piperidin-1-ylmethyl)-2-(1-methyl-1H-pyrrol-2-yl)-thieno[2,3-d]pyrimidin-4-ylamine

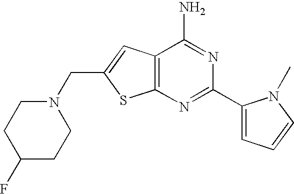

The title compound was prepared using N-Methylpyrrole-2-boronic acid, pinacol ester in place of 5-cyanothiophene-2-boronic acid as described in Example 85. 1H NMR (300 MHz, CHLOROFORM-D) δ ppm 7.05 (dd, J=3.8, 1.9 Hz, 1H), 6.89 (s, 1H), 6.73 (t, J=2.1 Hz, 1H), 6.17 (dd, J=3.8, 2.6 Hz, 1H), 5.25 (br s, 2H), 4.71 (d, J_{HF}=48.6 Hz, 1H), 4.09 (s, 3H), 3.72 (s, 2H), 2.58-2.68 (m, 2H), 2.45-2.53 (m, 2H), 1.84-1.99 (m, 4H); MS m/e 346 (M+H).

### Example 79

### 6-(4-Fluoro-piperidin-1-ylmethyl)-2-(2-isopropyl-phenyl)-thieno[2,3-d]pyrimidin-4-ylamine

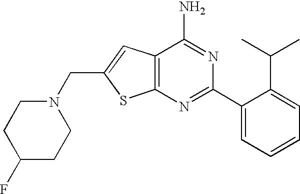

The title compound was prepared using 2-isopropyl phenylboronic acid in place of 5-cyanothiophene-2-boronic acid as described in Example 85. 1H NMR (300 MHz, CHLOROFORM-D) δ ppm 7.51 (d, J=7.2 Hz, 1H), 7.35-7.44 (m, 2H), 7.21-7.28 (m, 1H), 6.99 (s, 1H), 5.36 (s, 2H), 4.73 (d, J_{HF}=48.6 Hz, 1H), 3.77 (s, 2H), 3.44 (sept, J=6.9 Hz, 1H), 2.49-2.70 (m, 4H), 1.85-2.04 (m, 4H), 1.22 (d, J=6.8 Hz, 6H); MS m/e 385 (M+H).

### Example 80

### 6-(3,6-Dihydro-2H-pyridin-1-ylmethyl)-2-(3-methoxy-phenyl)-thieno[2,3-d]pyrimidin-4-ylamine

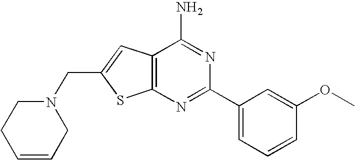

The title compound was prepared using 1,2,3,6-tetrahydropyridine and 3-methoxybenzonitrile in place of cis-2,6-dimethyl-piperidine and 5-tert-butyl-thiophene-2-carbonitrile, respectively, as described in Example 13. 1H NMR (300 MHz, CHLOROFORM-D) δ ppm 7.98-8.04 (m, 2H), 7.36 (t, J=7.9 Hz, 1H), 6.97-7.02 (m, 2H), 5.74-5.82 (m, 1H), 5.63-5.72 (m, 1H), 5.28 (br s, 2H), 3.91 (s, 3H), 3.84 (s, 2H), 3.05-3.14 (m, 2H), 2.65 (t, J=5.7 Hz, 2H), 2.14-2.24 (m, 2H); MS m/e 353 (M+H).

### Example 81

### 6-(4-Fluoro-piperidin-1-ylmethyl)-2-(1H-pyrrol-2-yl)-thieno[2,3-d]pyrimidin-4-ylamine

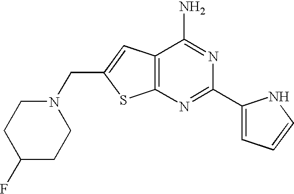

The title compound was prepared using 1-(tert-butoxycarbonyl)pyrrole-2-boronic acid in place of 5-cyanothiophene-2-boronic acid as described in Example 85. 1H NMR (400 MHz, CHLOROFORM-D) δ ppm 9.63 (br s, 1H), 7.09 (s, 2H), 6.95 (s, 1H), 6.31-6.38 (m, 1H), 4.77 (d, J_{HF}=48.4 Hz, 1H), 3.85 (s, 2H), 2.73 (br m, 4H), 1.96 (br m, 4H); MS m/e 332 (M+H).

### Example 82

### 3-[4-Amino-6-(4-fluoro-piperidin-1-ylmethyl)-thieno[2,3-d]pyrimidin-2-yl]-benzonitrile

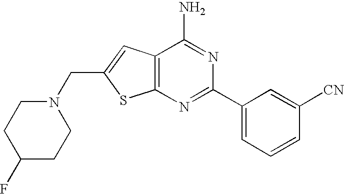

The title compound was prepared using 4-fluoropiperidine hydrochloride and 1,3-dicyanobenzene in place of cis-2,6-dimethyl-piperidine and 5-tert-butyl-thiophene-2-carbonitrile, respectively, as described in Example 13. 1H NMR (400 MHz, CHLOROFORM-D) δ ppm 8.77 (s, 1H), 8.68 (d, J=8.1 Hz, 1H), 7.70 (dt, J=7.7, 1.3 Hz, 1H), 7.56 (t, J=7.8 Hz, 1H), 7.00 (s, 1H), 5.26 (s, 2H), 4.73 (d, J_{HF}=48.7 Hz, 1H), 3.78 (s, 2H), 2.59-2.69 (m, 2H), 2.48-2.58 (m, 2H), 1.87-1.02 (m, 4H); MS m/e 368 (M+H).

### Example 83

### 3-(4-Amino-6-thiomorpholin-4-ylmethyl-thieno[2,3-d]pyrimidin-2-yl)-benzonitrile

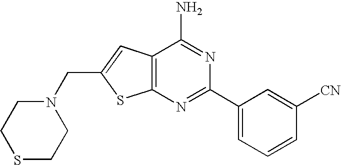

The title compound was prepared using thiomorpholine and 1,3-dicyanobenzene in place of cis-2,6-dimethyl-piperidine and 5-tert-butyl-thiophene-2-carbonitrile, respectively, as described in Example 13. 1H NMR (300 MHz, MeOD) δ ppm 8.61-8.67 (m, 2H), 7.93 (ddd, J=7.7, 1.3, 1.1 Hz, 1H), 7.70 (t, J=7.7 Hz, 1H), 7.60 (br s, 2H), 7.45 (s, 1H), 3.78 (s, 2H), 2.69-2.76 (m, 4H), 2.61-2.67 (m, 4H); MS m/e 368 (M+H).

### Example 84

### 2-(3-Methoxy-phenyl)-6-morpholin-4-ylmethyl-thieno[2,3-d]pyrimidin-4-ylamine

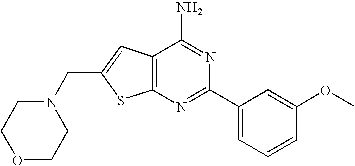

The title compound was prepared using morpholine and 3-methoxybenzonitrile in place of cis-2,6-dimethyl-piperidine and 5-tert-butyl-thiophene-2-carbonitrile, respectively, as described in Example 13. 1H NMR (300 MHz, CHLOROFORM-D) δ ppm 7.97-8.06 (m, 2H), 7.37 (t, J=7.9 Hz, 1H), 7.01 (dd, J=2.6, 0.8 Hz, 1H), 6.98 (s, 1H), 5.27 (br s, 2H), 3.91 (s, 3H), 3.71-3.77 (m, 6H), 2.52-2.57 (m, 4H); MS m/e 357 (M+H).

### Example 85

### 2-(3-Methoxy-phenyl)-6-thiomorpholin-4-ylmethyl-thieno[2,3-d]pyrimidin-4-ylamine

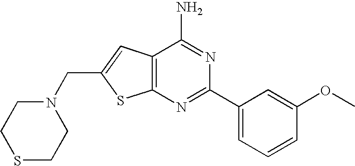

The title compound was prepared using thiomorpholine and 3-methoxybenzonitrile in place of cis-2,6-dimethyl-piperidine and 5-tert-butyl-thiophene-2-carbonitrile, respectively, as described in Example 13. 1H NMR (300 MHz, CHLOROFORM-D) δ ppm 7.97-8.04 (m, 2H), 7.37 (t, J=7.9 Hz, 1H), 7.00 (ddd, J=8.1, 2.6, 0.9 Hz, 1H), 6.95 (s, 1H), 5.26 (s, 2H), 3.91 (s, 3H), 3.76 (s, 2H), 2.76-2.84 (m, 4H), 2.67-2.74 (m, 4H); MS m/e 373 (M+H).

### Example 86

### 6-(3,3-Difluoro-piperidin-1-ylmethyl)-2-(3-methoxy-phenyl)-thieno[2,3-d]pyrimidin-4-ylamine

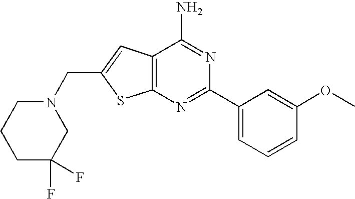

The title compound was prepared using 3,3-difluoropiperidine hydrochloride and 3-methoxybenzonitrile in place of cis-2,6-dimethyl-piperidine and 5-tert-butyl-thiophene-2-carbonitrile, respectively, as described in Example 13. 1H NMR (300 MHz, CHLOROFORM-D) δ ppm 7.97-8.05 (m, 2H), 7.37 (t, J=8.1 Hz, 1H), 6.96-7.03 (m, 2H), 5.32 (s, 2H), 3.91 (s, 3H), 3.85 (s, 2H), 2.74 (t, J_{HF}=11.1Hz, 2H), 2.49-2.60 (m, 2H), 1.73-1.98 (m, 4H); MS m/e 391 (M+H).

### Example 87

### 5-[4-Amino-6-(4-fluoro-piperidin-1-ylmethyl)-thieno[2,3-d]pyrimidin-2-yl]-nicotinonitrile

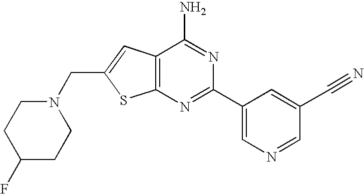

The title compound was prepared using 3-cyano-5-(4,4,5,5-tetramethyl-[1,3,2]dioxaborolan-2-yl)pyridine in place of 5-cyanothiophene-2-boronic acid as described in Example 85. 1H NMR (300 MHz, DMSO-D6) δ ppm 9.66 (d, J=1.9 Hz, 1H), 9.11 (d, J=1.9 Hz, 1H), 8.93 (t, J=2.1 Hz, 1H), 7.70 (br s, 2H), 7.47 (s, 1H), 4.72 (d, J_{HF}=49.0 Hz, 1H), 3.76 (s, 2H), 2.37-2.67 (m, 4H), 1.66-1.97 (m, 4H); MS m/e 369 (M+H).

### Example 88

### 4-[4-Amino-6-(2,5-dihydro-pyrrol-1-ylmethyl)-thieno[2,3-d]pyrimidin-2-yl]-benzonitrile

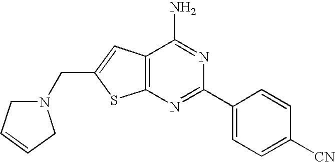

The title compound was prepared using 3-pyrroline and 1,4-dicyanobenzene in place of cis-2,6-dimethyl-piperidine and 5-tert-butyl-thiophene-2-carbonitrile, respectively, as described in Example 13. 1H NMR (300 MHz, CHLOROFORM-D) δ ppm 8.55 (d, J=8.7 Hz, 2H), 7.74 (d, J=8.7 Hz, 2H), 7.04 (s, 1H), 5.81 (s, 2H), 5.23 (br s, 2H), 4.09 (s, 2H), 3.61 (s, 4H); MS m/e 334 (M+H).

### Example 89

### 2-(5-Chloro-furan-2-yl)-6-(4-fluoro-piperidin-1-ylmethyl)-thieno[2,3-d]pyrimidin-4-ylamine hydrochloride

### Example 89

### Step a

### 4-Amino-2-furan-2-yl-thieno[2,3-d]pyrimidine-6-carbaldehyde

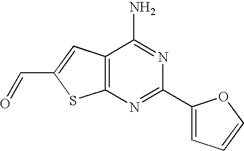

The title compound was prepared by using 2-furan-2-yl-6-methyl-thieno[2,3-d]pyrimidin-4-ylamine (prepared in example 1) in place of 2-(5-tert-butyl-thiophen-2-yl)-6-methyl-thieno[2,3-d]pyrimidin-4-ylamine as described in example 13.

### Example 89

### Step b

### 4-Amino-2-(5-chloro-furan-2-yl)-thieno[2,3-d]pyrimidine-6-carbaldehyde

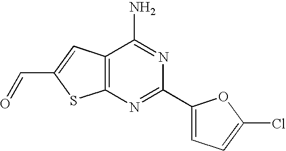

Solid NCS (196 mg, 1.5 mmol) was added to a THF solution (10 mL) of 4-amino-2-furan-2-yl-thieno[2,3-d]pyrimidine-6-carbaldehyde (300 mg, 1.2 mmol) and the mixture was heated to 50° C. After 16 h the mixture was diluted with EtOAc, washed with water and brine, dried (Na₂SO₄), and concentrated to give 325 mg of the title compound that was used without further purification.

### Example 89

### Step c

### 2-(5-Chloro-furan-2-yl)-6-(4-fluoro-piperidin-1-ylmethyl)-thieno[2,3-d]pyrimidin-4-ylamine hydrochloride

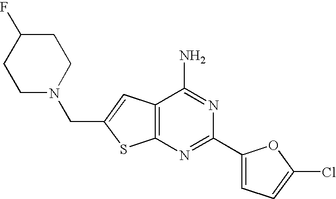

The title compound was prepared using 4-fluoropiperidine hydrochloride and 4-amino-2-(5-chloro-furan-2-yl)-thieno[2,3-d]pyrimidine-6-carbaldehyde in place of cis-2,6-dimethyl-piperidine and 4-amino-2-(5-tert-butyl-thiophen-2-yl)-thieno[2,3-d]pyrimidine-6-carbaldehyde, respectively, as described in Example 13. ¹H NMR (DMSO-d₆, 300 MHz): δ=7.72 (s, 1H), 7.24 (d, J=3.4 Hz, 1H), 6.70 (d, J=3.4 Hz, 1H), 4.64 (br. s., 2H), 3.35 (br. s., 1H), 3.16 (br. s., 4H), 2.08 ppm (br. s., 4H); MS m/e 367 (M+H)

### Example 90

### 2-(5-Chloro-furan-2-yl)-6-pyrrolidin-1-ylmethyl-thieno[2,3-d]pyrimidin-4-ylamine hydrochloride

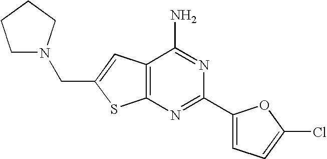

The title compound was prepared using pyrrolidine in place of 4-fluoropiperidine hydrochloride as described in Example 107. ¹H NMR (DMSO-d₆, 300 MHz): δ=7.65 (br. s., 1H), 7.18 (d, J=3.8 Hz, 1H), 6.67 (d, J=3.4 Hz, 1H), 3.99 (br. s., 2H), 2.68 (br. m, 4H), 1.66-1.89 (m, 4H); MS m/e 335 (M+H)

### Example 91

### 6-(Adamantan-1-ylaminomethyl)-2-furan-2-yl-thieno[2,3-d]pyrimidin-4-ylamine

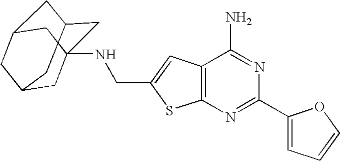

The title compound was prepared using 1-adamantylamine and 2-furonitrile in place of cis-2,6-dimethyl-piperidine and 5-tert-butyl-thiophene-2-carbonitrile, respectively, as described in Example 13. ¹H NMR (DMSO-d₆, 300 MHz): δ=9.98 (s, 1H), 8.52 (s, 1H), 8.13 (br. s., 2H), 7.92 (s, 1H), 7.29 (d, J=3.4 Hz, 1H), 6.70 (d, J=1.9 Hz, 1H), 4.30 (br. s., 2H), 1.75-1.91 (m, 8H), 1.69 (br. s., 4H), 1.57 ppm (br. s., 3H); MS m/e 381 (M+H)

### Example 92

### 6-(4-Fluoro-piperidin-1-ylmethyl)-2-furan-2-yl-thieno[2,3-d]pyrimidin-4-ylamine Hydrochloride

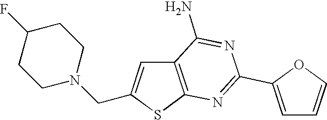

The title compound was prepared using 4-fluoropiperidine hydrochloride and 2-furonitrile in place of cis-2,6-dimethyl-piperidine and 5-tert-butyl-thiophene-2-carbonitrile, respectively, as described in Example 13. ¹H NMR (DMSO-d₆, 300 MHz): δ=7.93 (br. s., 1H), 7.72 (s, 1H), 7.24 (d, J=3.4 Hz, 1H), 6.70 (d, J=3.4 Hz, 1H), 4.64 (br. s., 2H), 3.35 (m., 1H), 3.16 (br. s., 4H), 2.08 ppm (br. s., 4H); MS m/e 333 (M+H)

### Example 93

### 6-Azepan-1-ylmethyl-2-(5-chloro-furan-2-yl)-thieno[2,3-d]pyrimidin-4-ylamine

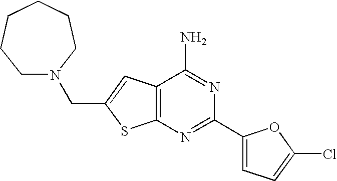

The title compound was prepared using hexamethyleneimine in place of 4-fluoropiperidine hydrochloride as described in Example 107. ¹H NMR (DMSO-d₆, 300 MHz): δ=7.74 (s, 1H), 7.24 (br. s., 1H), 6.79 (s, 1H), 4.61 (br. s., 2H), 4.50 (br. s., 2H), 3.38 (br. s., 2H), 3.13 (br. s., 4H), 1.84 (br. s., 4H), 1.63 (br. s., 4H); MS m/e 363 (M+H)

### Example 94

### 6-(3,3-Difluoro-pyrrolidin-1-ylmethyl)-2-furan-2-yl-thieno[2,3-d]pyrimidin-4-ylamine

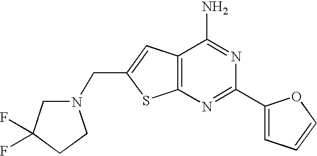

The title compound was prepared using 3,3-difluoro-pyrrolidine hydrochloride and 2-furonitrile in place of cis-2,6-dimethyl-piperidine and 5-tert-butyl-thiophene-2-carbonitrile, respectively, as described in Example 13. ¹H NMR (DMSO-d₆, 300 MHz): δ=7.82 (s, 1H), 7.54 (s, 2H), 7.42 (s, 1H), 7.12 (d, J=3.4 Hz, 1H), 6.63 (dd, J=3.4, 1.9 Hz, 1H), 3.88 (s, 2H), 2.97 (t, J=13.4 Hz, 2H), 2.80 (t, J=7.0 Hz, 2H), 2.13-2.40 ppm (m, 2H); MS m/e 337 (M+H)

### Example 95

### 2-(5-Chloro-furan-2-yl)-6-(3,6-dihydro-2H-pyridin-1-ylmethyl)-thieno[2,3-d]pyrimidin-4-ylamine

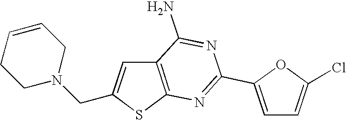

The title compound was prepared using 1,2,3,6-tetrahydropyridine in place of 4-fluoropiperidine hydrochloride as described in Example 107. ¹H NMR (CHLOROFORM-d, 300 MHz): δ=7.22 (d, J=3.4 Hz, 1H), 7.00 (s, 1H), 6.33 (d, J=3.4 Hz, 1H), 5.73-5.85 (m, 1H), 5.60-5.73 (m, 1H), 5.29 (br. s., 2H), 3.83 (s, 2H), 3.04-3.15 (m, 2H), 2.64 (t, J=5.8 Hz, 2H), 2.14-2.25 ppm (m, 2H); MS m/e 347 (M+H)

### Example 96

### 2-Cyclopropyl-6-(3,6-dihydro-2H-pyridin-1-ylmethyl)-thieno[2,3-d]pyrimidin-4-ylamine

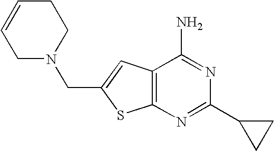

The title compound was prepared using 1,2,3,6-tetrahydropyridine and cyclopropylnitrile in place of cis-2,6-dimethyl-piperidine and 5-tert-butyl-thiophene-2-carbonitrile, respectively, as described in Example 13. ¹H NMR (DMSO-d₆, 300 MHz): δ=7.84 (s, 1H), 5.91 (br. s., 1H), 5.72 (br. s., 1H), 4.65 (br. s., 2H), 3.65 (br. s., 2H), 3.15 (br. s., 2H), 2.35 (m, 2H), 2.15 (br. s., 2H), 1.26 (m, 1H), 1.12 ppm (br. s., 4H); MS m/e 287 (M+H)

### Example 97

### 2-(5-Chloro-furan-2-yl)-6-(2,5-dihydro-pyrrol-1-ylmethyl)-thieno[2,3-d]pyrimidin-4-ylamine

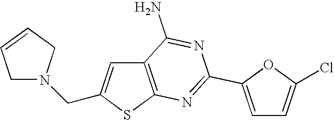

The title compound was prepared using 3-pyrroline in place of 4-fluoropiperidine hydrochloride as described in Example 107. ¹H NMR (DMSO-d₆, 300 MHz): δ=7.45 (s, 1H), 7.19 (d, J=3.0 Hz, 1H), 6.85 (s, 2H), 6.67 (d, J=3.0 Hz, 1H), 5.37 (s, 2H), 4.28 ppm (br. s., 6H); MS m/e 333 (M+H)

### Example 98

### 6-(3,6-Dihydro-2H-pyridin-1-ylmethyl)-2-furan-2-yl-thieno[2,3-d]pyrimidin-4-ylamine

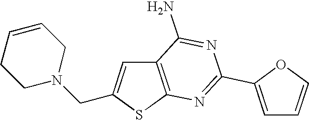

The title compound was prepared using 1,2,3,6-tetrahydropyridine and 2-furonitrile in place of cis-2,6-dimethyl-piperidine and 5-tert-butyl-thiophene-2-carbonitrile, respectively, as described in Example 13. ¹H NMR (CHLOROFORM-d, 300 MHz): δ=7.60 (s, 1H), 7.25 (s, 1H), 7.00 (s, 1H), 6.55 (dd, J=3.4, 1.9 Hz, 1H), 5.77 (br. s., 1H), 5.69 (br. s., 1H), 5.25 (br. s., 2H), 3.83 (s, 2H), 2.99-3.16 (m, 2H), 2.64 (t, J=5.7 Hz, 2H), 2.12-2.26 ppm (m, 2H); MS m/e 313 (M+H)

### Example 99

### 2-(5-Difluoromethyl-furan-2-yl)-6-(4,4-difluoro-piperidin-1-ylmethyl)-thieno[2,3-d]pyrimidin-4-ylamine

### Example 99

### Step a

### 5-Difluoromethyl-furan-2-carbonitrile

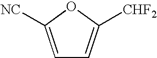

To a solution of Et₂NSF₃ (2.8 mL, 21.4 mmol) and CH₂Cl₂ (10 mL) at 4° C. was added a solution of 5-formyl-furan-2-carbonitrile (2.44 g, 20.2 mmol; W. Hoyle and G. P. Roberts, J. Med. Chem. 1973, 16, 709) in CH₂Cl₂ (10 mL). After 30 min at 4° C., saturated aqueous NaHCO₃ was added, the layers were separated and the aqueous layer was extracted with CH₂Cl₂. The combined organics were dried (Na₂SO₄) and concentrated to give 2.15 g of 5-difluoromethyl-furan-2-carbonitrile that was used without further purification.

### Example 99

### Step b

### 2-(5-Difluoromethyl-furan-2-yl)-6-(4,4-difluoro-piperidin-1-ylmethyl)-thieno[2,3-d]pyrimidin-4-ylamine

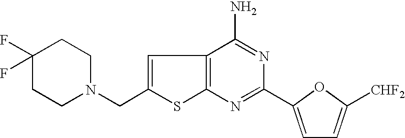

The title compound was prepared using 4,4-difluoropiperidine hydrochloride and 5-difluoromethyl-furan-2-carbonitrile in place of cis-2,6-dimethyl-piperidine and 5-tert-butyl-thiophene-2-carbonitrile, respectively, as described in Example 13. ¹H NMR (400 MHz, Acetone-d₆) δ=7.41 (s, 1H), 7.21 (d, J=3.4 Hz, 1H), 7.01 (t, J=53.7 Hz, 1H), 6.94-6.99 (m, 1H), 6.92 (br. s, 2H), 3.87 (d, J=1.0 Hz, 2H), 2.66 (t, J=5.5 Hz, 4H), 1.95-2.04 (m, 4H); MS m/e 401 (M+H).

### Example 100

### 2-(5-Difluoromethyl-furan-2-yl)-6-(4-fluoro-piperidin-1-ylmethyl)-thieno[2,3-d]pyrimidin-4-ylamine

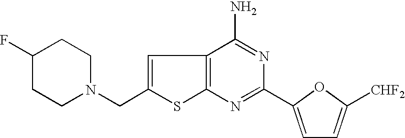

The title compound was prepared using 4-fluoropiperidine hydrochloride in place of 4,4-difluoropiperidine hydrochloride as described in Example 119. ¹H NMR (400 MHz, Acetone-d₆) δ=7.39 (s, 1H), 7.20 (d, J=3.7 Hz, 1H), 7.01 (t, J=53.7 Hz, 1H), 6.94-6.98 (m, 1H), 6.89 (br. s., 2H), 3.78 (d, J=1.2 Hz, 2H), 2.61-2.71 (m, 2H), 2.43-2.52 (m, 2H), 2.08-2.10 (m, 1H), 1.74-1.99 (m, 4H); MS m/e 383 (M+H).

### Example 101

### 2-(5-Difluoromethyl-furan-2-yl)-6-(3,3-difluoro-piperidin-1-ylmethyl)-thieno[2,3-d]pyrimidin-4-ylamine

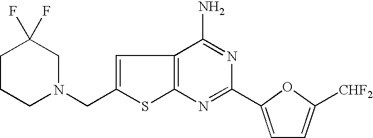

The title compound was prepared using 3,3-difluoropiperidine hydrochloride in place of 4,4-difluoropiperidine hydrochloride as described in Example 119. ¹H NMR (400 MHz, Acetone-d₆) δ=7.41 (s, 1H), 7.21 (d, J=3.7 Hz, 1H), 7.01 (t, J=53.7 Hz, 1H), 6.89-6.98 (m, 3H), 3.90 (s, 2H), 2.77 (t, J=11.5 Hz, 2H), 2.58 (t, J=5.0 Hz, 2H), 1.85-1.98 (m, 2H), 1.71-1.81 (m, 2H); MS m/e 401 (M+H).

### Example 102

### 2-(5-Difluoromethyl-furan-2-yl)-6-(2,6-dimethyl-piperidin-1-ylmethyl)-thieno[2,3-d]pyrimidin-4-ylamine

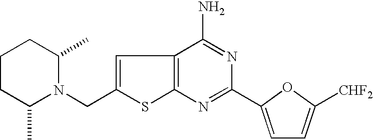

The title compound was prepared using cis-2,6-dimethyl-piperidine in place of 4,4-difluoropiperidine hydrochloride as described in Example 119. ¹H NMR (400 MHz, Acetone-d₆) δ=7.40 (s, 1H), 7.19 (d, J=3.4 Hz, 1H), 7.00 (t, J=53.7 Hz, 1H), 6.93-6.97 (m, 1H), 6.89 (br. s., 1H), 4.08 (s, 2H), 2.50-2.62 (m, 2H), 1.53-1.67 (m, 4H), 1.27-1.33 (m, 2H), 1.15 (d, J=6.4 Hz, 6H); MS m/e 393 (M+H).

### Example 103

### 6-Diethylaminomethyl-2-(5-difluoromethyl-furan-2-yl)-thieno[2,3-d]pyrimidin-4-ylamine

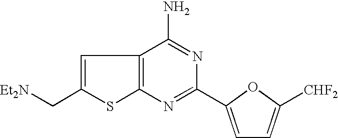

The title compound was prepared using diethylamine in place of 4,4-difluoropiperidine hydrochloride as described in Example 119. ¹H NMR (300 MHz, CDCl₃) δ=7.27 (s, 1H), 6.97 (s, 1H), 6.80-6.85 (m, 1H), 6.78 (t, J=54.3 Hz, 1H), 6.52 (br. s., 2H), 3.85 (s, 2H), 2.61 (q, J=7.2 Hz, 4H), 1.08 (t, J=7.0 Hz, 6H); MS m/e 353 (M+H).

### Example 104

### 2-(2-Chloro-pyridin-4-yl)-6-piperidin-1-ylmethyl-thieno[2,3-d]pyrimidin-4-ylamine

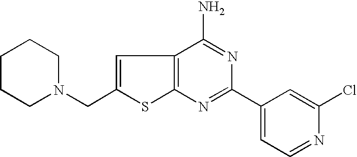

The title compound was prepared using piperidine and 2-chloro-isonicotinonitrile in place of cis-2,6-dimethyl-piperidine and 5-tert-butyl-thiophene-2-carbonitrile, respectively, as described in Example 13. ¹H NMR (300 MHz, DMSO-d₆) δ=8.53 (d, J=5.3 Hz, 1H), 8.13-8.29 (m, 2H), 7.68 (s, 2H), 7.46 (s, 1H), 3.70 (s, 2H), 2.91-3.11 (m, 2H), 2.29-2.45 (m, 4H), 1.34-1.58 (m, 4H); MS m/e 360/362 (M+H).

### Example 105

### 2-(2-Chloro-pyridin-4-yl)-6-morpholin-4-ylmethyl-thieno[2,3-d]pyrimidin-4-ylamine

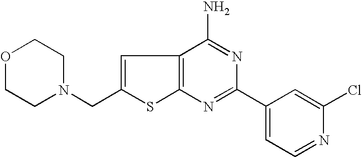

The title compound was prepared using morpholine and 2-chloro-isonicotinonitrile in place of cis-2,6-dimethyl-piperidine and 5-tert-butyl-thiophene-2-carbonitrile, respectively, as described in Example 13. ¹H NMR (300 MHz, CDCl₃) δ=8.48 (d, J=5.3 Hz, 1H), 8.34 (s, 1H), 8.21 (dd, J=1.5, 5.3 Hz, 1H), 7.05 (s, 1H), 5.36 (br. s., 2H), 3.70-3.86 (m, 6H), 2.47-2.65 (m, 4H); MS m/e 362/364 (M+H).

### Example 106

### 3-(4-Amino-6-morpholin-4-ylmethyl-thieno[2,3-d]pyrimidin-2-yl)-phenol

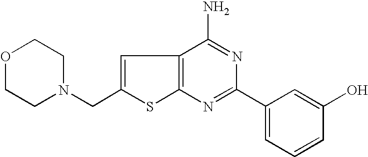

The title compound was prepared using morpholine and 3-hydroxy-benzonitrile in place of cis-2,6-dimethyl-piperidine and 5-tert-butyl-thiophene-2-carbonitrile, respectively, as described in Example 13. ¹H NMR (300 MHz, Acetone-d₆) δ=8.36 (br. s., 1H), 7.99 (s, 1H), 7.96 (d, J=7.9 Hz, 1H), 7.38 (s, 1H), 7.26 (t, J=7.7 Hz, 1H), 6.91 (dd, J=2.6, 7.9 Hz, 1H), 6.74 (br. s., 2H), 3.76 (s, 2H), 3.64 (t, J=4.5 Hz, 4H), 2.38-2.59 (m, 4H); MS m/e 343 (M+H).

### Example 107

### 2-(5-Difluoromethyl-furan-2-yl)-6-morpholin-4-ylmethyl-thieno[2,3-d]pyrimidin-4-ylamine

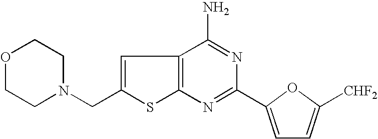

The title compound was prepared using morpholine in place of 4,4-difluoropiperidine hydrochloride as described in Example 119. ¹H NMR (400 MHz, DMSO-d₆) δ=7.63 (br. s., 2H), 7.43 (s, 1H), 7.19 (d, J=3.4 Hz, 1H), 7.16 (t, J=53.3 Hz, 1H), 7.02 (m, 1H), 3.72 (s, 2H), 3.59 (t, J=4.4 Hz, 4H), 2.44 (m, 4H); MS m/e 367 (M+H).

### Biological Assays and Activity

### Ligand Binding Assay for Adenosine A2a Receptor

Ligand binding assay of adenosine A2a receptor was performed using plasma membrane of HEK293 cells containing human A2a adenosine receptor (PerkinElmer, RB-HA2a) and radioligand [³H]CGS21680 (PerkinElmer, NET 1021). Assay was set up in 96-well polypropylene plate in total volume of 200 μL by sequentially adding 20 μL 1:20 diluted membrane, 130 μL assay buffer (50 mM Tris.HCl, pH7.4 10 mM MgCl₂, 1 mM EDTA) containing [³H] CGS21680, 50 μL diluted compound (4×) or vehicle control in assay buffer. Nonspecific binding was determined by 80 mM NECA. Reaction was carried out at room temperature for 2 hours before filtering through 96-well GF/C filter plate pre-soaked in 50 mM Tris.HCl, pH7.4 containing 0.3% polyethylenimine. Plates were then washed 5 times with cold 50 mM Tris.HCl, pH7.4, dried and sealed at the bottom. Microscintillation fluid 30 μL was added to each well and the top sealed. Plates were counted on Packard Topcount for [³H]. Data was analyzed in Microsoft Excel and GraphPad Prism programs. (Varani, K.; Gessi, S.; Dalpiaz, A.; Borea, P. A. British Journal of Pharmacology, 1996, 117, 1693)

### Adenosine A2a Receptor Functional Assay (A2AGAL2)

To initiate the functional assay, cryopreserved CHO-K1 cells overexpressing the human adenosine A2a receptor and containing a cAMP inducible beta-galactosidase reporter gene were thawed, centrifuged, DMSO containing media removed, and then seeded with fresh culture media into clear 384-well tissue culture treated plates (BD #353961) at a concentration of 10K cells/well. Prior to assay, these plates were cultured for two days at 37° C., 5% CO₂, 90% Rh. On the day of the functional assay, culture media was removed and replaced with 45 uL assay medium (Hams/F-12 Modified (Mediatech # 10-080CV) supplemented w/0.1% BSA). Test compounds were diluted and 11 point curves created at a 1000× concentration in 100% DMSO. Immediately after addition of assay media to the cell plates, 50 nL of the appropriate test compound antagonist or agonist control curves were added to cell plates using a Cartesian Hummingbird. Compound curves were allowed to incubate at room temperature on cell plates for approximately 15 minutes before addition of a 15 nM NECA (Sigma E2387) agonist challenge (5 uL volume). A control curve of NECA, a DMSO/Media control, and a single dose of Forskolin (Sigma F3917) were also included on each plate. After additions, cell plates were allowed to incubate at 37° C., 5% CO₂, 90% Rh for 5.5-6 hours. After incubation, media was removed, and cell plates were washed 1×50 uL with DPBS w/o Ca & Mg (Mediatech 21-031-CV). Into dry wells, 20 uL of 1× Reporter Lysis Buffer (Promega E3971 (diluted in dH₂O from 5× stock)) was added to each well and plates frozen at −20° C. overnight. For β-galactosidase enzyme calorimetric assay, plates were thawed out at room temperature and 20 μL 2× assay buffer (Promega) was added to each well. Color was allowed to develop at 37° C., 5% CO₂, 90% Rh for 1-1.5 h or until reasonable signal appeared. The calorimetric reaction was stopped with the addition of 60 μL/well 1M sodium carbonate. Plates were counted at 405 nm on a SpectraMax Microplate Reader (Molecular Devices). Data was analyzed in Microsoft Excel and IC/EC50 curves were fit using a standardized macro.

### Adenosine A1 Receptor Functional Assay (A1GAL2)

To initiate the functional assay, cryopreserved CHO-K1 cells overexpressing the human adenosine A1 receptor and containing a cAMP inducible beta-galactosidase reporter gene were thawed, centrifuged, DMSO containing media removed, and then seeded with fresh culture media into clear 384-well tissue culture treated plates (BD #353961) at a concentration of 10K cells/well. Prior to assay, these plates were cultured for two days at 37° C., 5% CO₂, 90% Rh. On the day of the functional assay, culture media was removed and replaced with 45 uL assay medium (Hams/F-12 Modified (Mediatech # 10-080CV) supplemented w/0.1% BSA). Test compounds were diluted and 11 point curves created at a 1000× concentration in 100% DMSO. Immediately after addition of assay media to the cell plates, 50 mL of the appropriate test compound antagonist or agonist control curves were added to cell plates using a Cartesian Hummingbird. Compound curves were allowed to incubate at room temperature on cell plates for approximately 15 minutes before addition of a 4 nM r-PIA (Sigma P4532)/1 uM Forskolin (Sigma F3917) agonist challenge (5 uL volume). A control curve of r-PIA in 1 uM Forskolin, a DMSO/Media control, and a single dose of Forskolin were also included on each plate. After additions, cell plates were allowed to incubate at 37° C., 5% CO₂, 90% Rh for 5.5-6 hours. After incubation, media was removed, and cell plates were washed 1×50 uL with DPBS w/o Ca & Mg (Mediatech 21-031-CV).

Into dry wells, 20 uL of 1× Reporter Lysis Buffer (Promega E3971 (diluted in dH₂O from 5× stock)) was added to each well and plates frozen at −20° C. overnight. For β-galactosidase enzyme calorimetric assay, plates were thawed out at room temperature and 20 μL 2× assay buffer (Promega) was added to each well. Color was allowed to develop at 37° C., 5% CO₂, 90% Rh for 1-1.5 h or until reasonable signal appeared. The calorimetric reaction was stopped with the addition of 60 μL/well 1M sodium carbonate. Plates were counted at 405 nm on a SpectraMax Microplate Reader (Molecular Devices). Data was analyzed in Microsoft Excel and IC/EC50 curves were fit using a standardized macro.

A2a ASSAY DATA

| Example | A2AGAL2 Ki μM | A2A-B Ki μM | A1GAL2 Ki μM |
|---|---|---|---|
| 1 | ND | ND | ND |
| 2 | ND | ND | ND |
| 3 | ND | ND | ND |
| 4 | ND | ND | ND |
| 5 | ND | ND | ND |
| 6 | ND | ND | ND |
| 7 | 0.0183251 | 0.2024437 | 0.698433 |
| 8 | 0.0781628 | ND | >0.610098 |
| 9 | 0.0893717 | ND | 0.296893 |
| 10 | 0.0173061 | ND | 0.143781 |
| 11 | 0.0248886 | ND | 0.194133 |
| 12 | 0.114051 | ND | 0.332659 |
| 13 | >2.33938 | ND | >0.92747 |
| 14 | >2.33938 | ND | >0.92747 |
| 15 | 0.207683 | ND | >0.92747 |
| 16 | 1.01158 | ND | 0.677954 |
| 17 | 0.150349 | ND | 1.07152 |
| 18 | 0.130227 | ND | 0.504197 |
| 19 | 0.0120282 | ND | 0.0396187 |
| 20 | 0.153922 | ND | 0.761553 |
| 21 | >1.36082 | ND | >0.593882 |
| 22 | 0.958297 | ND | >1.06832 |
| 23 | 0.0644614 | ND | 0.463767 |
| 24 | ND | ND | ND |
| 25 | 0.245301 | ND | 2.73401 |
| 26 | ND | 0.207491 | ND |
| 27 | ND | ND | ND |
| 28 | 0.0280608 | 0.0144212 | 6.43428 |
| 29 | 0.0362159 | 0.0933469 | 1.89496 |
| 30 | 0.114604 | 0.139091 | >10 |
| 31 | 0.0552332 | 0.13671 | 2.13059 |
| 32 | 0.168035 | 0.1424839 | 2.47971 |
| 33 | ND | 0.215824 | ND |
| 34 | 0.0284053 | 0.0186681 | 0.605759 |
| 35 | 0.0220141 | 0.0150003 | 0.800018 |
| 36 | ND | 0.371278 | ND |
| 37 | ND | ND | ND |
| 38 | 0.0318713 | 0.0327416 | 1.05439 |
| 39 | 0.0259478 | 0.0108543 | 0.62044 |
| 40 | ND | 0.374628 | ND |
| 41 | 0.202162 | 0.186509 | 3.31971 |
| 42 | 0.053039 | 0.0880643 | 18.20878 |
| 43 | 0.403832 | ND | >10 |
| 44 | 0.00564807 | 0.0013999 | 0.099793 |
| 45 | 0.229087 | 0.19002 | 2.71894 |
| 46 | 0.142988 | 0.089002 | 3.55386 |
| 47 | 0.349301 | ND | 2.30356 |
| 48 | 0.733331 | ND | 0.85546 |
| 49 | 0.1166 | ND | 0.785417 |
| 50 | 0.360662 | ND | 2.89468 |
| 51 | 0.00979942 | 0.0059007 | 1.33906 |
| 52 | 0.297303 | 0.219989 | 4.00313 |
| 53 | 0.0122999 | 0.0139991 | 0.655843 |
| 54 | 0.0115001 | 0.0081997 | 0.878214 |
| 55 | 1.05925 | ND | 3.4135 |
| 56 | 0.0486968 | 0.0209991 | 2.22844 |
| 57 | 0.00662369 | 0.0084004 | 0.294036 |
| 58 | 0.0735868 | ND | 0.331207 |
| 59 | 0.348097 | ND | 0.63387 |
| 60 | 0.300331 | ND | 1.25026 |
| 61 | 0.123254 | ND | 0.616311 |
| 62 | 0.0502805 | 0.383972 | 1.18114 |
| 63 | 0.177582 | 0.150003 | 1.63155 |
| 64 | 0.0834833 | 0.0769662 | 2.06538 |
| 65 | >10 | 0.0417638 | 0.129449 |
| 66 | 1.3938 | ND | 8.42753 |
| 67 | 0.383707 | ND | 8.5546 |
| 68 | 0.0132556 | 0.377833 | 0.211934 |
| 69 | 0.238451 | 1.01789 | 3.89493 |
| 70 | 0.240325 | 0.393007 | 2.96415 |
| 71 | 0.302831 | ND | 3.22181 |
| 72 | 0.0913061 | ND | 1.90634 |
| 73 | 1.02873 | 2.02116 | 5.04778 |
| 74 | 0.0358674 | 0.0932396 | 1.00531 |
| 75 | 0.356205 | ND | 3.24639 |
| 76 | 0.209315 | ND | 0.739605 |
| 77 | 0.0950167 | 0.0749204 | 2.1737 |
| 78 | 0.634308 | 0.733162 | 1.59845 |
| 79 | 1.0361 | 7.41481 | 4.46992 |
| 80 | 0.102282 | 0.183696 | 1.07696 |
| 81 | 0.795243 | ND | 3.89135 |
| 82 | 0.0537527 | 0.443608 | 2.94578 |
| 83 | 0.0649083 | 0.28132 | 1.77746 |
| 84 | 0.372649 | ND | 2.98951 |
| 85 | 0.169239 | 0.659326 | 2.95937 |
| 86 | 0.0974989 | 0.241824 | 1.80884 |
| 87 | 6.62217 | ND | >10 |
| 88 | 0.0788679 | 0.0759976 | 2.35342 |
| 89 | 0.0358922 | 0.0019002 | 1.07944 |
| 90 | 0.0222792 | 0.0037 | 0.712033 |
| 91 | 0.120531 | 0.0150003 | 1.02991 |
| 92 | 0.0189496 | 0.0119591 | 0.74114 |
| 93 | 0.0331971 | 0.0018001 | 1.42495 |
| 94 | 0.00822622 | ND | 1.23509 |
| 95 | 0.0107424 | 0.0034002 | 0.187629 |
| 96 | 0.324639 | 1.61994 | 3.01717 |
| 97 | 0.0102 | 0.00054 | 0.783069 |
| 98 | 0.008852 | 0.0203236 | 0.1584335 |
| 99 | 0.61038 | ND | 17.8238 |
| 100 | 0.0424326 | ND | 1.67456 |
| 101 | 0.0425109 | ND | 2.10426 |
| 102 | 0.0276248 | ND | 0.732487 |
| 103 | 0.130918 | ND | 2.0917 |
| 104 | 0.691194 | ND | 3.85923 |
| 105 | 0.12314 | 0.934114 | 0.875588 |
| 106 | 0.815831 | ND | 2.43781 |
| 107 | 0.0304719 | 0.0661454 | 2.70147 |

ND indicates no data was available

While the foregoing specification teaches the principles of the present invention, with examples provided for the purpose of illustration, it will be understood that the practice of the invention encompasses all of the usual variations, adaptations and/or modifications as come within the scope of the following claims and their equivalents.

All publications disclosed in the above specification are hereby incorporated by reference in full.

We claim:

1. A compound of Formula A 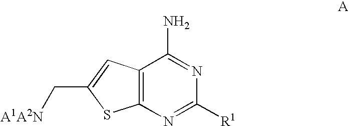
wherein:
R¹ is cyclopropyl, benzo[1,3]dioxolyl, or an aromatic ring selected from the group consisting of phenyl, fluorophenyl, and heteroaryl, wherein said aromatic ring is optionally substituted with one substituent selected from the group consisting of: —OH, OC₍₁₋₄₎alkyl, Cl, Br, —CN, F, CHF₂, C₍₁₋₄₎alkyl, and cyclopropyl;
A¹ is H or —C₍₁₋₄₎alkyl;
A² is —C₍₁₋₄₎alkyl, —C₍₁₋₆₎cycloalkyl, —CH₂CH₂OR^{a}, —COR^{a}, heteroaryl, adamantyl, or phenyl, wherein said heteroaryl or phenyl is optionally substituted with up to three substituents selected from the group consisting of Cl, F, Br, OC₍₁₋₄₎alkyl, OCF₃, C₍₁₋₄₎alkyl, and C(O)C₍₁₋₄₎alkyl;
alternatively, A¹ and A² may be taken together with their attached nitrogen to form a heterocyclic ring selected from the group consisting of: 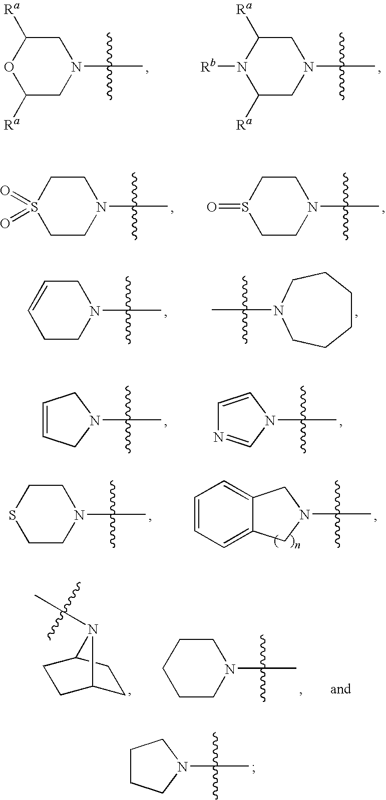
wherein said 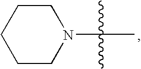
and said 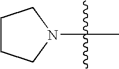
are optionally substituted with R^{a}, R^{c}, oxo, phenyl, or CH₂OC₍₁₋₄₎alkyl;
wherein:
n is 1 or 2;
R¹ is H, CF₃, OH, F, or C₍₁₋₄₎alkyl;
R^{b} is H, —C₍₁₋₄₎alkyl, or —C(O)C₍₁₋₄₎alkyl; and
R^{c} is H or F;
and solvates, hydrates, and pharmaceutically acceptable salts thereof.

2. A compound of claim 1, wherein:
R¹ is cyclopropyl, furyl, thiazolyl, thiophenyl, oxazolyl, isoxazolyl, pyridyl, benzo[1,3]dioxolyl, pyrrolyl, benzofuranyl, fluorophenyl, or phenyl, wherein said furyl, thiazolyl, thiophenyl, oxazolyl, isoxazolyl, pyridyl, benzo[1,3]dioxolyl, pyrrolyl, benzofuranyl, or phenyl is optionally substituted with OH, OC₍₁₋₄₎alkyl, Cl, Br, —CN, F, CHF₂, OCF₃, C₍₁₋₄₎alkyl, or cyclopropyl;
and solvates, hydrates, and pharmaceutically acceptable salts thereof.

3. A Compound of claim 2, wherein:
A¹ is H or —C₍₁₋₄₎alkyl;
A² is —C₍₁₋₄₎alkyl, —C₍₁₋₆₎cycloalkyl, —CH₂CH₂OR^{a}, —COR^{a}, pyridyl, adamantyl, or phenyl, wherein said heteroaryl or phenyl is optionally substituted with up to three substituents selected from the group consisting of Cl, F, Br, OC₍₁₋₄₎alkyl, OCF₃, C₍₁₋₄₎alkyl, and C(O)C₍₁₋₄₎alkyl;
alternatively, A¹ and A² may be taken together with their attached nitrogen to form a heterocyclic ring selected from the following: 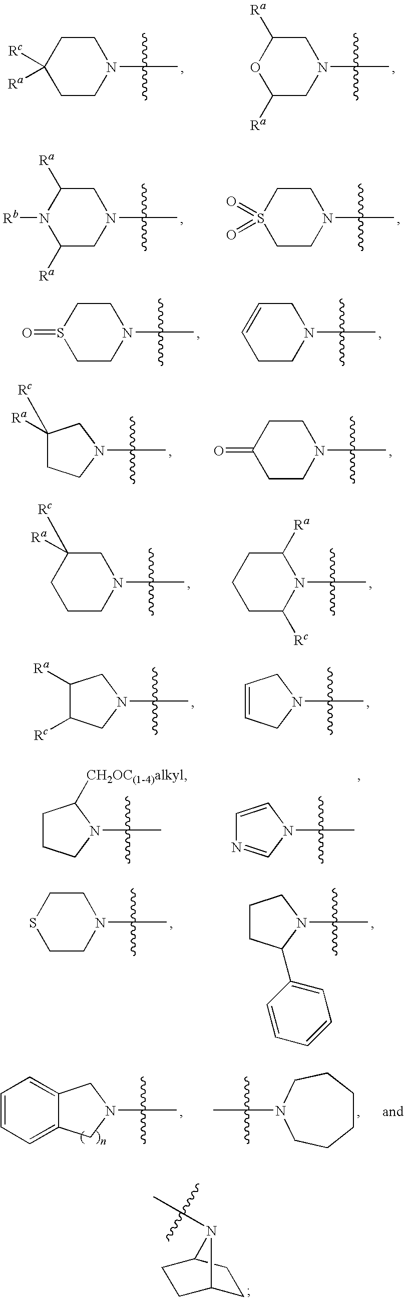
wherein:
n is 1 or 2;
R^{a} is H, CF₃, OH, F, or C₍₁₋₄₎alkyl;
R^{b} is H, —C₍₁₋₄₎alkyl, or —C(O)C₍₁₋₄₎alkyl; and
R^{c} is H or F;
and solvates, hydrates, and pharmaceutically acceptable salts thereof.

4. A compound of claim 3, wherein
R¹ is cyclopropyl, furyl, thiazolyl, thiophenyl, oxazolyl, isoxazolyl, pyridyl, benzo[1,3]dioxolyl, pyrrolyl, benzofuranyl, fluorophenyl, or phenyl, wherein said furyl, thiazolyl, thiophenyl, oxazolyl, isoxazolyl, pyridyl, benzo[1,3]dioxolyl, pyrrolyl, benzofuranyl, fluorophenyl, or phenyl is optionally substituted with OH, OCH₃, Cl, Br, —CN, F, CHF₂, OCF₃, CH₃, CH₂CH₃, CH(CH₃)₂, C(CH₃)₃, or cyclopropyl;
A¹ is H, or C₍₁₋₄₎alkyl;
A² is C₍₁₋₄₎alkyl, —CH₂CH₂OCH₃, cyclopropyl, adamantyl, or cyclohexyl;
alternatively, A¹ and A² may be taken together with their attached nitrogen to form a heterocyclic ring selected from the following: 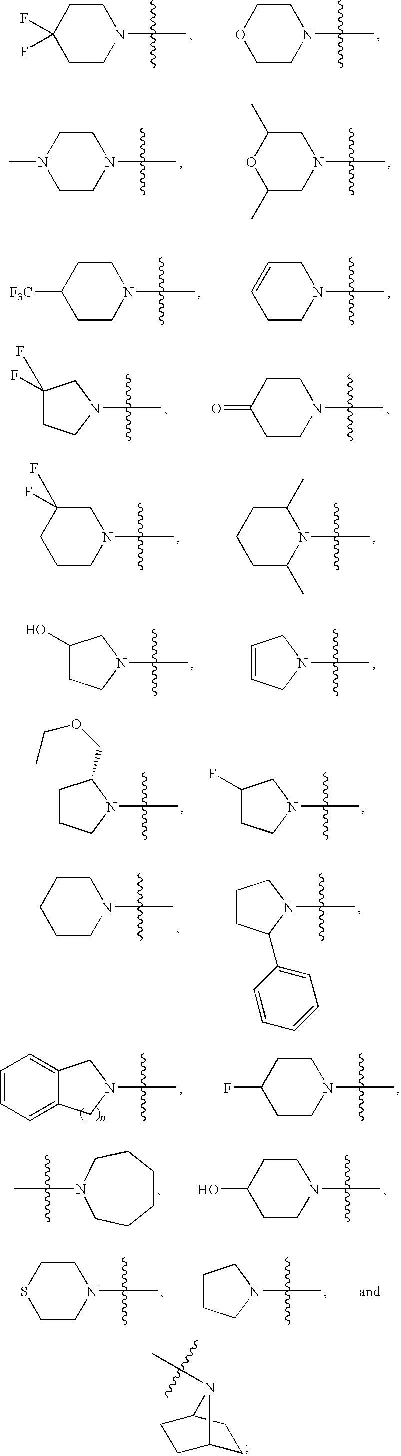
wherein n is 1 or 2;
and solvates, hydrates, and pharmaceutically acceptable salts thereof.

5. A compound of claim 4, wherein
R¹ is cyclopropyl; furyl, wherein said furyl is optionally substituted with Cl, Br, cyclopropyl, CH₃, CH₂CH₃, CHF₂, or CH(CH₃)₂; thiazolyl, wherein said thiazolyl is optionally substituted with CH₃; thiophenyl, wherein said thiophenyl is optionally substituted with C(CH₃)₃, or —CN; oxazolyl; isoxazolyl; pyridyl, wherein said pyridyl is substituted with —CN, or Cl; benzo[1,3]dioxolyl, pyrrolyl, wherein said pyrrolyl is optionally substituted with CH₃; benzofuranyl, fluorophenyl, wherein said fluorophenyl is optionally substituted with F; or phenyl, wherein said phenyl is substituted with CN, Cl, OCH₃, CON(CH₃)₂, CH(CH₃)₂, or OH;
A¹ is H, —CH₃, or —CH₂CH₃;
A² is —CH₃, —CH₂CH₃, —CH₂CH₂OCH₃, cyclopropyl, adamantyl, or cyclohexyl;
alternatively, A¹ and A² may be taken together with their attached nitrogen to form a heterocyclic ring selected from the following: 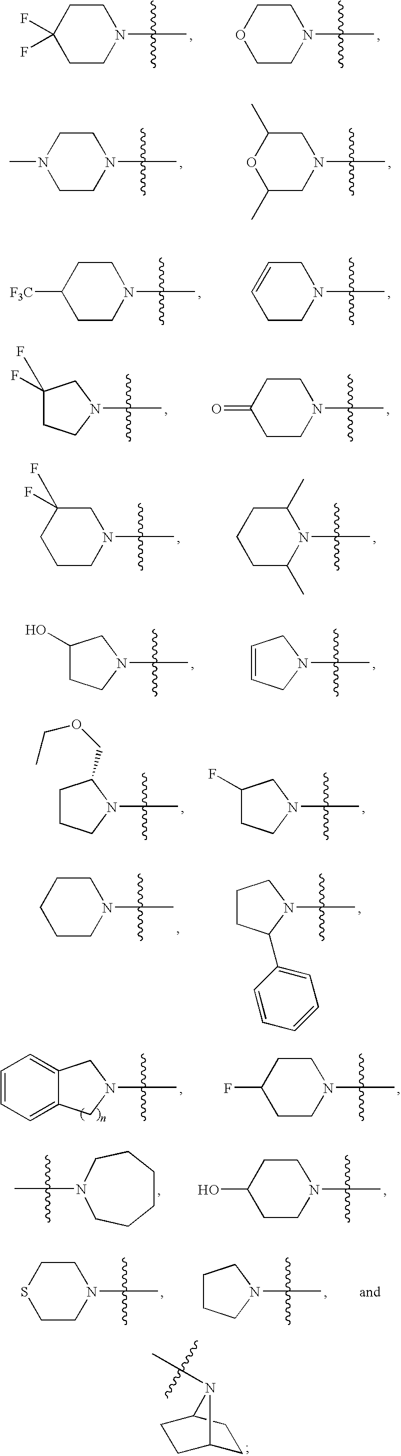
wherein n is 1 or 2;
and solvates, hydrates, and pharmaceutically acceptable salts thereof.

6. A compound selected from the group consisting of: 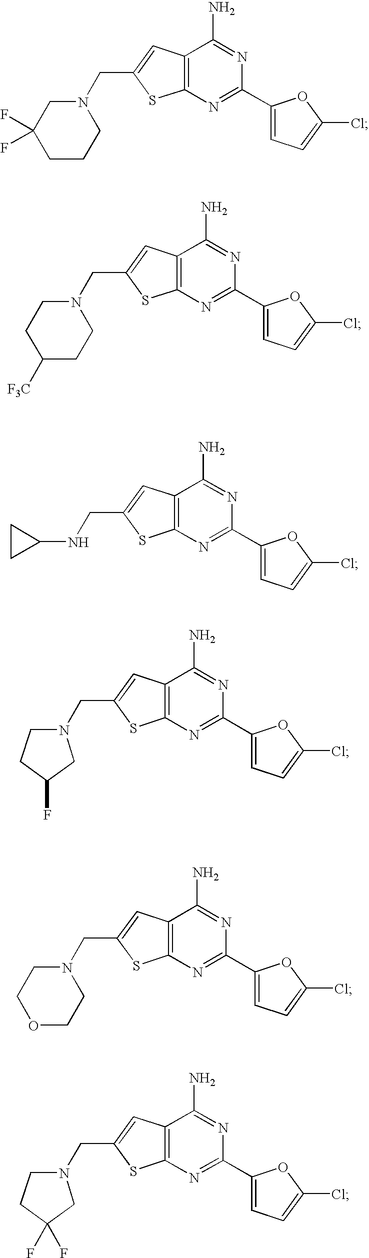 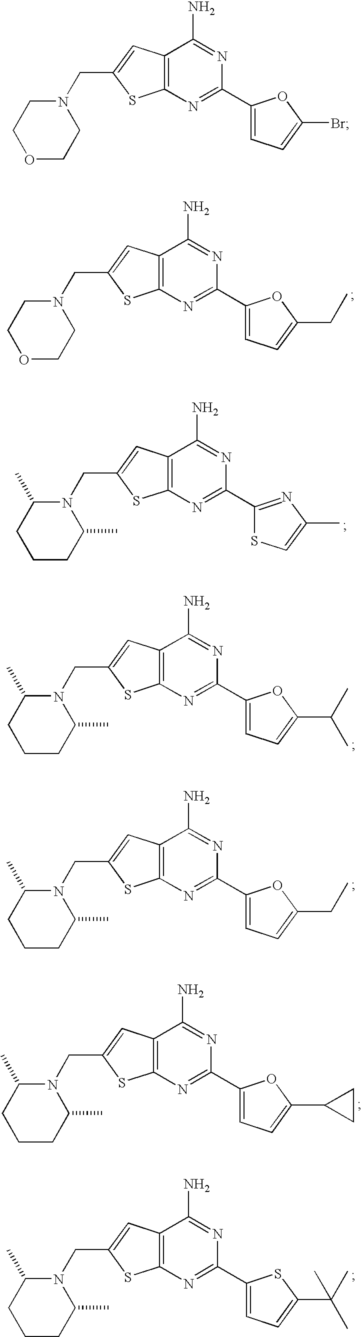 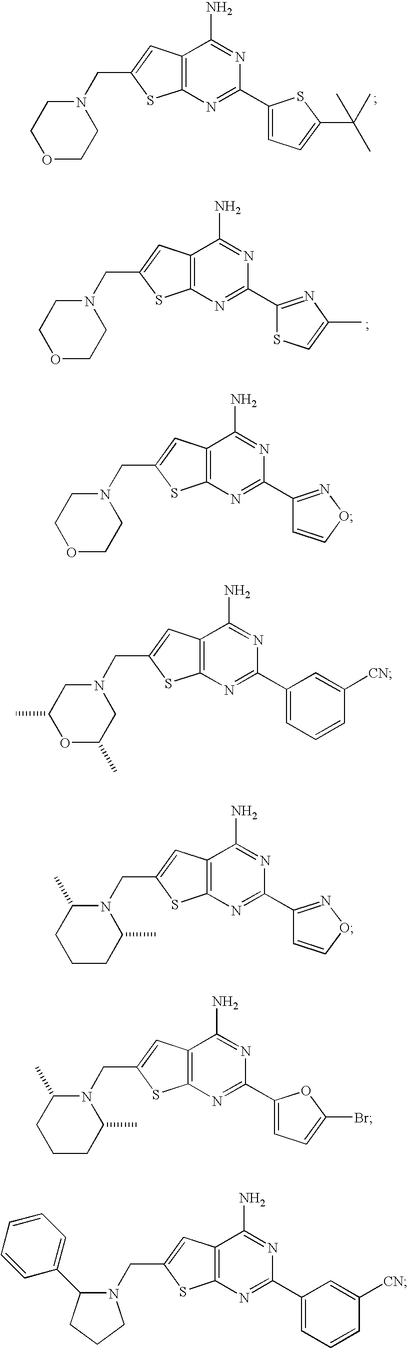 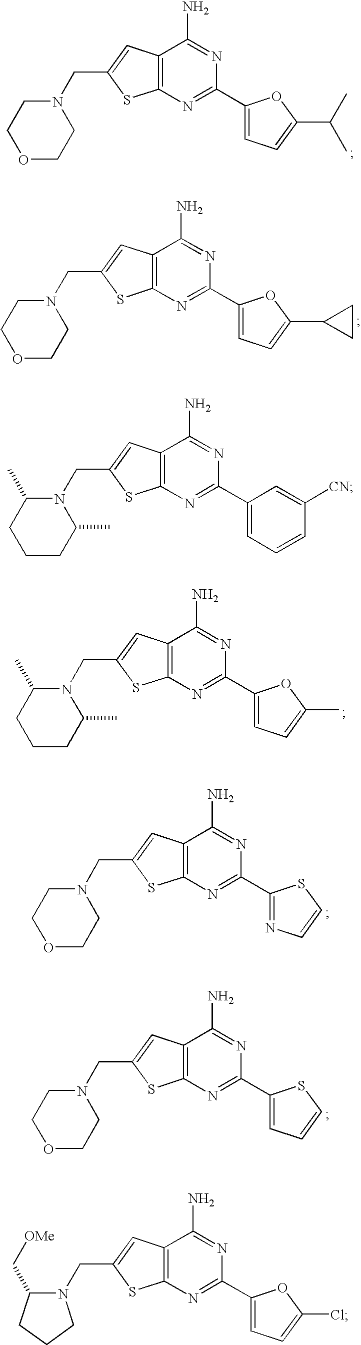 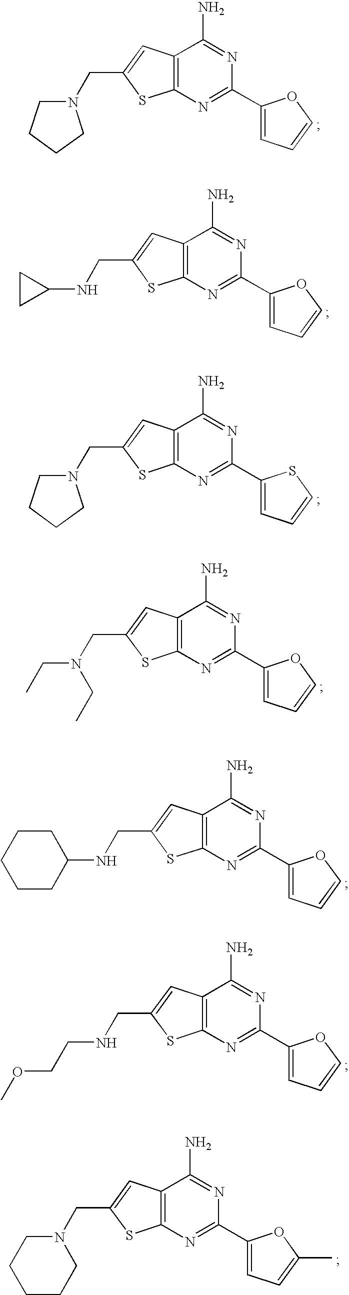 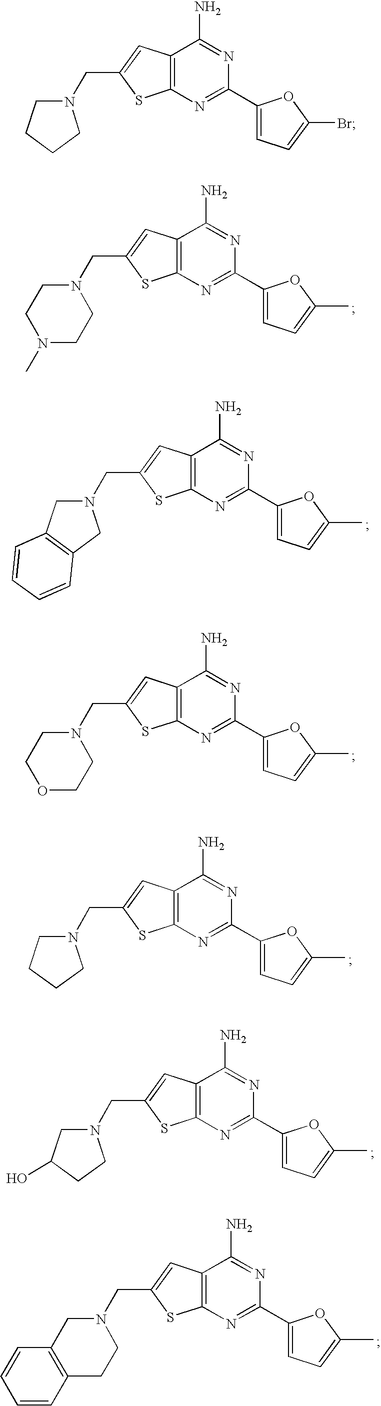 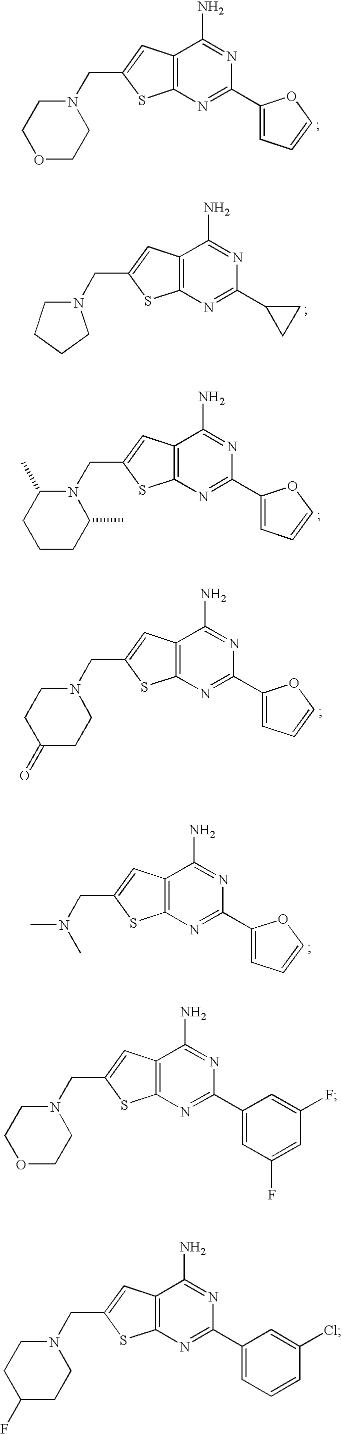 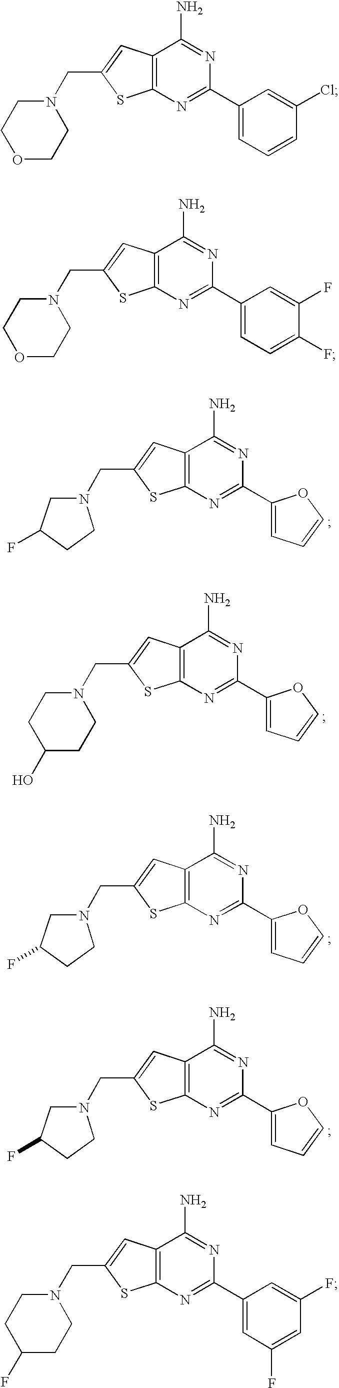 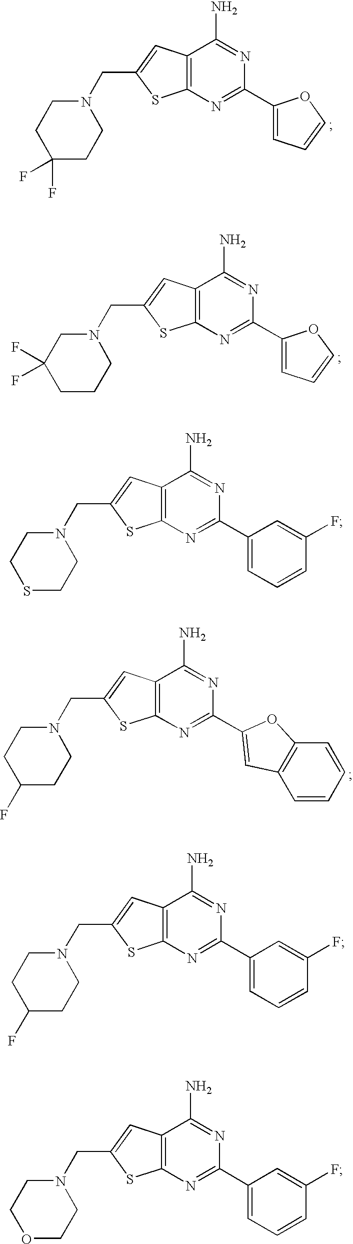 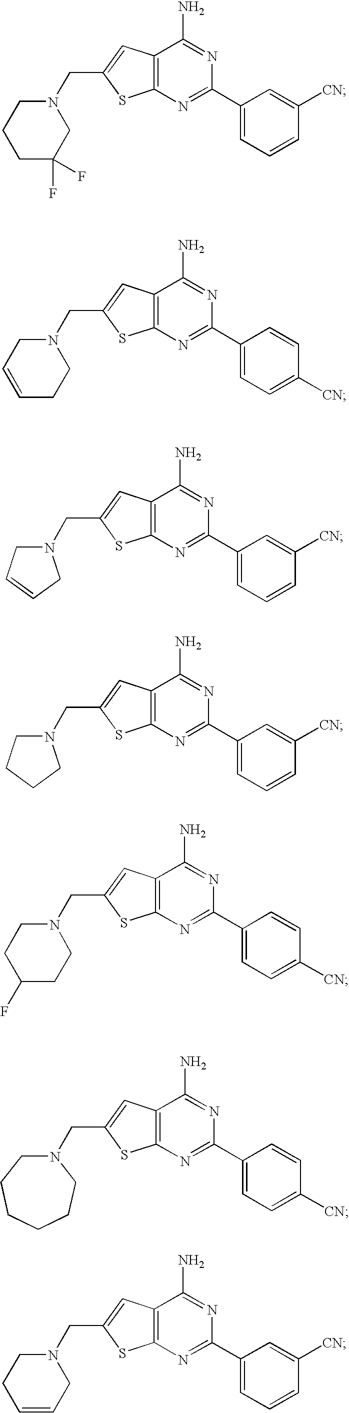 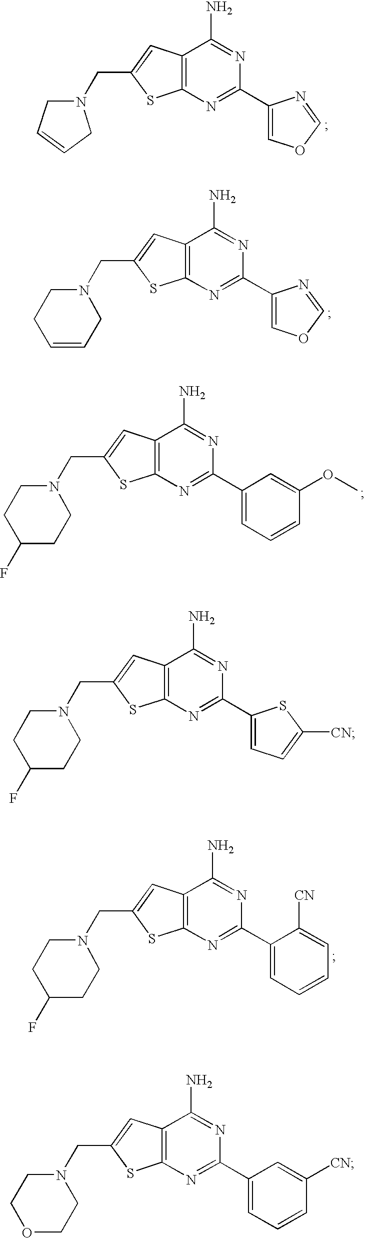 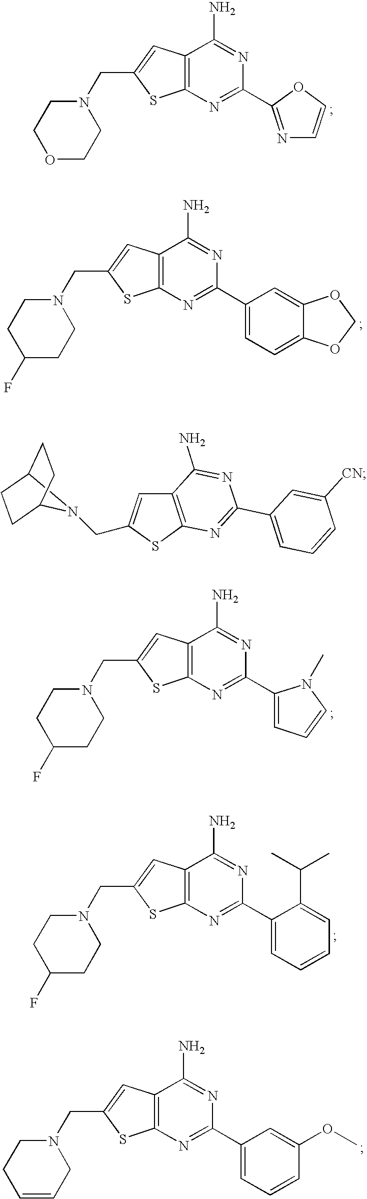 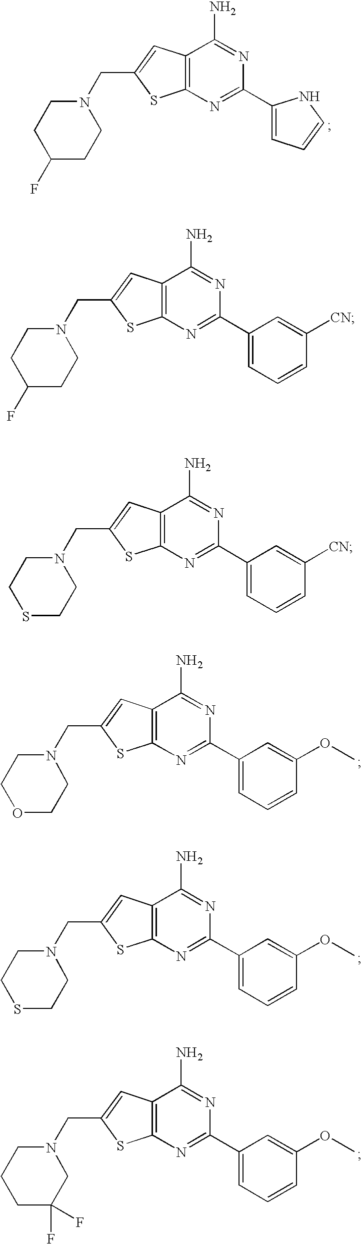 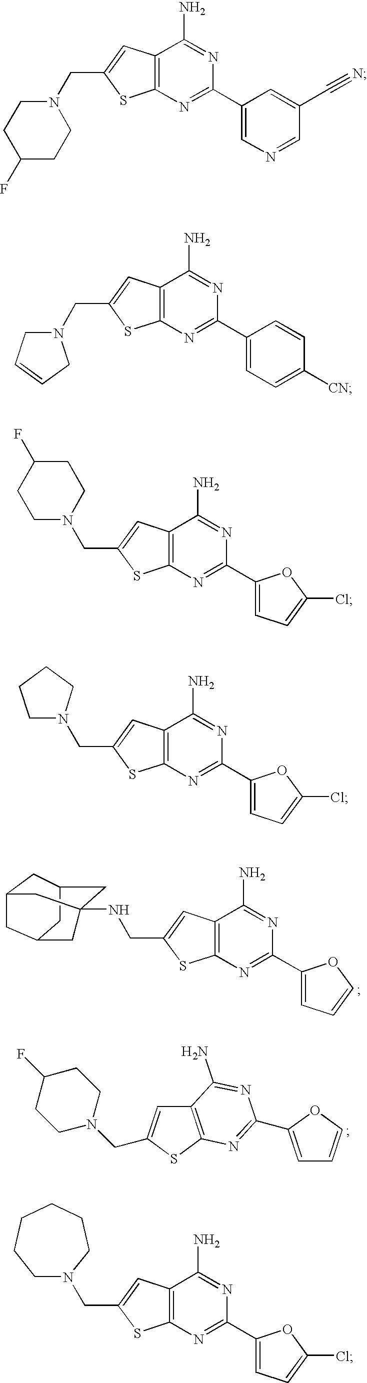 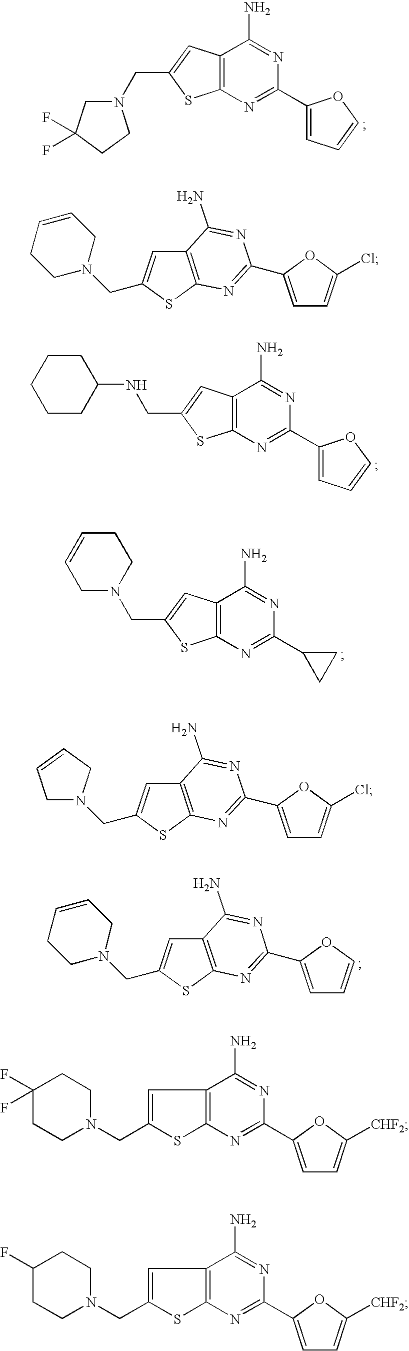 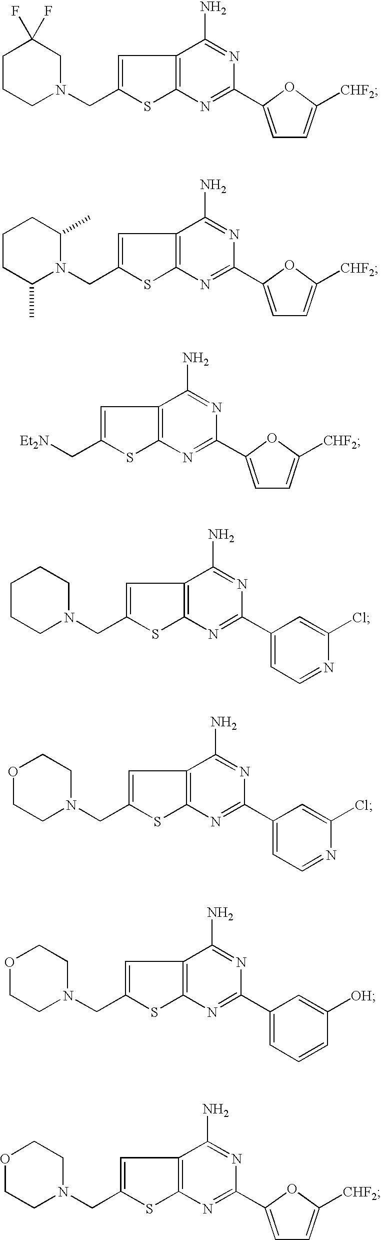
and solvates, hydrates, and pharmaceutically acceptable salts thereof.

7. A pharmaceutical composition comprising the compound of claim 1; and a pharmaceutically acceptable carrier.

8. A method of treating a subject having a disorder ameliorated by antagonizing Adenosine A2a receptors in appropriate cells in the subject, which comprises administering to the subject a therapeutically effective dose of the compound of claim 1.

9. A method of preventing a disorder ameliorated by antagonizing Adenosine A2a receptors in appropriate cells in the subject, comprising administering to the subject a prophylactically effective dose of the compound of claim 1 either preceding or subsequent to an event anticipated to cause a disorder ameliorated by antagonizing Adenosine A2a receptors in appropriate cells in the subject.

10. The method of claim 8 comprising administering to the subject a therapeutically or prophylactically effective dose of the pharmaceutical composition of claim 7.

11. The method of claim 9 comprising administering to the subject a therapeutically or prophylactically effective dose of the pharmaceutical composition of claim 7.

12. The method of claim 8, wherein the disorder is a neurodegenerative disorder or a movement disorder.

13. The method of claim 8, wherein the disorder is selected from the group consisting of Parkinson's Disease, Huntington's Disease, Multiple System Atrophy, Corticobasal Degeneration, Alzheimer's Disease, and Senile Dementia.

14. The method of claim 9, wherein the disorder is a neurodegenerative disorder or a movement disorder.

15. The method of claim 9, wherein the disorder is selected from the group consisting of Parkinson's Disease, Huntington's Disease, Multiple System Atrophy, Corticobasal Degeneration, Alzheimer's Disease, and Senile Dementia.

16. The method of claim 8, wherein the disorder is Parkinson's Disease.

17. The method of claim 8, where the disorder is addiction.

18. The method of claim 8, wherein the disorder is Attention Deficit Hyperactivity Disorder (ADHD).

19. The method of claim 8, wherein the disorder is depression.

20. The method of claim 8, wherein the disorder is anxiety.
